SEQUENCE LISTING (44 sequences)

SEQ ID NO: 1 - DNA, 2187 bp, Homo sapiens
  source 1..2187: mol_type = genomic DNA
atggcggggttctgggtcgggacagcaccgctggtcgctgccggacggcgtgggcggtggccgccgcagcagctgatgctgagcgcggcgctgcggaccctgaagcatgttctgtactattcaagacagtgcttaatggtgtcccgtaatcttggttcagtgggatatgatcctaatgaaaaaacttttgataaaattcttgttgctaatagaggagaaattgcatgtcgggttattagaacttgcaagaagatgggcattaagacagttgccatccacagtgatgttgatgctagttctgttcatgtgaaaatggcggatgaggctgtctgtgttggcccagctcccaccagtaaaagctacctcaacatggatgccatcatggaagccattaagaaaaccagggcccaagctgtacatccaggttatggattcctttcagaaaacaaagaatttgccagatgtttggcagcagaagatgtcgttttcattggacctgacacacatgctattcaagccatgggcgacaagattgaaagcaaattattagctaagaaagcagaggttaatacaatccctggctttgatggagtagtcaaggatgcagaagaagctgtcagaattgcaagggaaattggctaccctgtcatgatcaaggcctcagcaggtggtggtgggaaaggcatgcgcattgcttgggatgatgaagagaccagggatggttttagattgtcatctcaagaagctgcttctagttttggcgatgatagactactaatagaaaaatttattgataatcctcgtcatatagaaatccaggttctaggtgataaacatgggaatgctttatggcttaatgaaagagagtgctcaattcagagaagaaatcagaaggtggtggaggaagcaccaagcatttttttggatgcggagactcgaagagcgatgggagaacaagctgtagctcttgccagagcagtaaaatattcctctgctgggaccgtggagttccttgtggactctaagaagaatttttatttcttggaaatgaatacaagactccaggttgagcatcctgtcacagaatgcattactggcctggacctagtccaggaaatgatccgtgttgctaagggctaccctctcaggcacaaacaagctgatattcgcatcaacggctgggcagttgaatgtcgggtttatgctgaggacccctacaagtcttttggtttaccatctattgggagattgtctcagtaccaagaaccgttacatctacctggtgtccgagtggacagtggcatccaaccaggaagtgatattagcatttattatgatcctatgatttcaaaactaatcacatatggctctgatagaactgaggcactgaagagaatggcagatgcactggataactatgttattcgaggtgttacacataatattgcattacttcgagaggtgataatcaactcacgctttgtaaaaggagacatcagcactaaatttctctccgatgtgtatcctgatggcttcaaaggacacatgctaaccaagagtgagaagaaccagttattggcaatagcatcatcattgtttgtggcattccagttaagagcacaacattttcaagaaaattcaagaatgcctgttattaaaccagacatagccaactgggagctctcagtaaaattgcatgataaagttcataccgtagtagcatcaaacaatgggtcagtgttctcggtggaagttgatgggtcgaaactaaatgtgaccagcacgtggaacctggcttcgcccttattgtctgtcagcgttgatggcactcagaggactgtccagtgtctttctcgagaagcaggtggaaacatgagcattcagtttcttggtacagtgtacaaggtgaatatcttaaccagacttgccgcagaattgaacaaatttatgctggaaaaagtgactgaggacacaagcagtgttctgcgttccccgatgcccggagtggtggtggccgtctctgtcaagcctggagacgcggtagcagaaggtcaagaaatttgtgtgattgaagccatgaaaatgcagaatagtatgacagctgggaaaactggcacggtgaaatctgtgcactgtcaagctggagacacagttggagaaggggatctgctcgtggagctggaatga

SEQ ID NO: 2 - DNA, 2187 bp, synthetic construct
  source 1..2187: mol_type = other DNA
atggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtga

SEQ ID NO: 3 - DNA, 2187 bp, synthetic construct
  misc_feature 1..2187: note = Synthetic construct
  source 1..2187: mol_type = other DNA
atggccgggttttgggtgggcacggccccgctcgtagcagctggcaggcgggggcgatggcccccccagcagcttatgcttagtgccgccttgcggacgctgaagcacgtcctttactactctagacagtgccttatggtaagccgaaatttgggaagtgtaggttatgatcccaacgagaagacctttgataagatactggttgctaaccgaggggagatagcgtgtcgagttattcgcacctgtaagaagatgggaattaaaaccgtggccatccatagcgatgtcgacgcttccagtgtgcacgttaaaatggccgacgaggccgtatgcgtggggcctgcccctacctctaagtcatacctgaacatggatgcaattatggaagctattaagaagactcgggcgcaggctgtccaccctggatatggatttctttctgagaataaggagtttgcccggtgtctggcggcagaagacgtcgtattcattggaccggatacgcacgctatccaagccatgggagataagatcgagagcaagctcctggctaagaaagctgaagtgaacaccattcctggctttgacggcgtggtgaaggacgcagaggaagctgttcgcatcgcccgcgaaattggatatcccgtgatgataaaagcatctgcgggggggggcgggaagggcatgagaattgcctgggatgatgaagaaactagagatggtttccgcttgtcttctcaggaagccgcatcatcctttggagatgaccgattgctcatagagaaatttatcgacaatccacggcatattgagatccaagtgcttggcgacaagcacggtaacgcgctttggctcaacgaacgagagtgttcaatccagaggaggaaccagaaggttgtagaagaagcaccatctattttcctcgacgcagaaactcggcgggctatgggggaacaagcagtggcactggctcgagccgttaaatattctagtgcggggacagtagaattcctcgtagatagcaagaagaatttttattttcttgagatgaatacgcgccttcaagtggaacacccagtcacggaatgtataactggccttgacttggttcaggagatgatacgggtggctaagggttatcctcttcggcataagcaggctgatattcgcataaatgggtgggcggtcgagtgcagagtttatgctgaggacccatacaagtcattcggacttccttctataggcagactgtcacaatatcaagagccacttcatctcccaggtgtaagagtagattccggaatacaacctggctccgatatatctatttactatgatccaatgattagtaagttgattacatatgggagtgatcggaccgaagctttgaagcggatggcggacgcgctggataactacgtgataaggggtgtcacgcacaatatagctctgctgagggaggtaattatcaacagtcggttcgtgaagggtgacattagcactaagttcctctccgacgtgtacccagacggttttaaagggcacatgcttactaagtccgaaaagaatcaactgttggctattgcgtcttccctttttgttgctttccaactgcgcgcgcagcatttccaggagaatagcagaatgcccgttatcaaacctgatattgcgaactgggaattgtcagttaagctgcatgataaggtgcataccgtagtggctagtaataacggaagcgttttttccgttgaagtagacggctccaagcttaatgtgacgagcacatggaaccttgcctctccactgcttagtgtgagtgtggacggaacgcagaggacagttcaatgcctgagtcgggaagcgggaggtaacatgagtatacaattcctcggaaccgtctataaagttaacattttgacgagattggcggctgaactgaataagttcatgctcgagaaagtgactgaggacacttcaagcgtactgaggagccctatgccgggggttgtcgtagcagtgtctgttaagccaggagatgcggtggcagaaggccaagaaatttgcgtgattgaggcaatgaaaatgcagaactcaatgaccgccggaaaaacgggcacggtcaaatctgtgcattgtcaggcaggcgacacagtcggcgagggtgatctcctggtagagttggaatga

SEQ ID NO: 4 - DNA, 2187 bp, synthetic construct
  misc_feature 1..2187: note = Synthetic construct
  source 1..2187: mol_type = other DNA
atggccggcttctgggtggggactgctccccttgtcgccgcaggacgcagaggccgctggcctcctcagcagctcatgctctcagcagctctgaggaccctgaaacacgtgctttactacagtcgacagtgtctgatggtgtctaggaacctgggtagcgtgggctatgatcccaatgaaaagacctttgacaaaatactggtcgctaatagaggggaaattgcttgtcgcgtgatacggacgtgcaagaagatgggtatcaaaaccgtggcaattcactctgacgttgatgcttcctcagtgcatgtaaagatggcggatgaggctgtttgcgtgggtccagcacctacaagcaagagctatctcaacatggatgccatcatggaagctatcaagaaaacccgtgcacaagctgtgcatccagggtatggctttctctccgagaacaaagaatttgcccggtgtctggcagcggaggacgtggtgttcattgggcctgatacgcatgcaattcaagccatgggcgataagattgagagcaagctgcttgctaagaaagcagaagttaacacaatcccaggctttgacggcgttgtcaaagacgccgaagaagcggtacgtattgcccgagaaatcggctaccccgttatgatcaaggcgtcagccggaggtggaggaaaagggatgaggattgcctgggatgacgaggagactagggatgggttccggctctccagtcaggaagcagcatcttcttttggtgacgatagactgctgatagagaaattcatcgacaaccctcgacacattgaaatccaggtactgggagacaaacacggaaatgcactttggctcaatgaacgcgagtgctccattcagcgcaggaaccagaaagtggtcgaggaagcaccctccatcttcctggatgccgagacaaggcgcgctatgggcgagcaggccgttgcactcgctagagccgtgaagtactcttctgcgggtaccgtggaatttctggtagacagcaagaagaacttctatttcctggagatgaatacccggctgcaagtcgagcatccagtcactgagtgtataactggcctggacctggtacaggaaatgattcgtgtagcgaagggatacccgctccggcacaaacaagccgacattcgcatcaatgggtgggctgtggagtgcagagtctatgcagaggatccctataagtccttcgggcttccctccataggcaggcttagtcagtatcaggagccattgcacttgcctggcgtcagggtggactccggcatccaaccgggcagcgacatttcaatttactacgatcccatgatcagcaagttgattacctatggatctgaccggacagaggctctgaagagaatggccgacgccctggacaattacgtgataagaggagtgacacacaacattgccctgttgcgggaggtgatcatcaatagcagattcgtgaagggtgacatctccaccaagttcctgagtgacgtataccccgacggctttaaggggcatatgctgacaaagtcagagaagaatcaactcctcgcaatagccagtagcctgtttgttgccttccagctgagggctcagcacttccaggagaatagcagaatgcccgttatcaaacctgatatcgcgaattgggaattgagcgtgaagctgcacgataaagttcatactgttgtggcctcaaacaatggaagcgtctttagcgtggaggtcgatggatccaaactgaacgtgaccagtacctggaatttggccagtccgctgttgtctgtctccgtggatggaacgcaacgaactgtgcagtgtctgtctcgcgaagccggaggcaacatgagcattcagtttctcgggactgtgtacaaagtcaacatcctgacccgactggctgccgagctgaacaaatttatgcttgagaaagtcactgaggatacgtctagcgtccttcggagtcctatgccaggggtggtggtggccgtttcagtcaaaccaggtgatgccgtagccgaaggtcaggaaatctgcgttatcgaggctatgaagatgcagaacagcatgacagccgggaaaaccggaacagtgaagtcagttcattgccaggctggggacacagtcggcgagggcgatttgctggtggaactggaatga

SEQ ID NO: 5 - DNA, 2187 bp, synthetic construct
  misc_feature 1..2187: note = Synthetic construct
  source 1..2187: mol_type = other DNA
atggccggattttgggttggaacagctcctctggtggccgctgggagaagaggaagatggcctcctcagcagctgatgctgtctgccgctctgagaaccctgaaacacgtgctgtactacagccggcagtgcctgatggtgtccagaaatctgggcagcgtgggctacgaccccaacgagaaaaccttcgacaagatcctggtggccaaccggggagagatcgcctgcagagtgatccggacctgcaagaagatgggcatcaagaccgtggccatccactccgatgtggatgcctctagcgtgcacgtgaaaatggccgatgaggccgtgtgtgtgggccctgctcctacaagcaagagctacctgaacatggacgccatcatggaagccattaagaaaacaagagcccaggccgtgcatcccggctacggatttctgagcgagaacaaagaatttgcccggtgcctggccgccgaggacgtggtgtttattggccctgatacacacgccatccaggccatgggcgataagatcgagtctaagctgctggccaagaaagccgaagtgaacacaatccccggcttcgacggcgtggtcaaggatgctgaagaagccgtgcggatcgccagagaaatcggctaccccgtgatgatcaaagcctctgctggcggaggcggcaagggaatgagaatcgcctgggacgacgaagagacacgcgacggctttagactgagcagccaagaagccgccagctccttcggagatgacagactgctgatcgagaagttcatcgacaaccccagacacatcgagatccaggtgctgggcgacaagcacggaaatgccctgtggctgaacgagagagagtgcagcatccagcggcggaaccagaaagtggtggaagaggcccctagcatcttcctggacgccgaaactcggagagccatgggagaacaggctgtggctctggctagagccgtgaagtatagcagcgccggcaccgtggaatttctggtggacagcaagaagaacttctacttcctcgagatgaacacccggctgcaggtcgagcaccctgtgaccgagtgtatcacaggcctggacctggtgcaagagatgatcagagtggccaagggctaccctctgagacacaagcaggccgacatccggatcaatggctgggccgttgagtgcagagtgtacgccgaggatccctacaagagcttcggcctgcctagcatcggccggctgtctcagtatcaagagccactgcatctgcccggcgtcagagtggattctggaatccagcctggcagcgacatcagcatctactacgaccctatgatctccaagctgatcacctacggcagcgaccggacagaggccctgaagagaatggctgacgccctggacaactacgtgatcagaggcgtgacccacaatatcgccctgctgcgggaagtgatcatcaacagcagattcgtgaaaggcgatatcagcaccaagtttctgtccgacgtgtaccccgacggcttcaagggacacatgctgaccaagagcgagaagaaccagctgctcgccattgcctccagcctgtttgtggcctttcagctgagagcccagcacttccaagagaacagcagaatgcccgtgatcaagcccgatatcgccaactgggagctgagcgtgaagctgcacgataaggtgcacacagtggtggccagcaacaacggctccgtgttcagcgtggaagtggacggcagcaagctgaacgtgacctccacctggaatctggcctctccactgctgtccgtgtctgtggatggcacccagagaaccgtgcagtgtctgagcagagaagcaggcggcaatatgagcatccagtttctgggcaccgtgtacaaagtgaacatcctgaccagactggccgctgagctgaacaagttcatgctggaaaaagtgaccgaggacaccagcagcgtgctgagatctcctatgcctggtgtcgtggtggccgtgtcagtgaaacctggggatgctgtggccgagggccaagagatctgtgtgatcgaggccatgaagatgcagaacagcatgaccgccggcaagaccggcacagtgaagtctgtgcattgtcaggccggcgatacagtcggagaaggcgatctgctggtggaactggaatga

SEQ ID NO: 6 - DNA, 2187 bp, synthetic construct
  misc_feature 1..2187: note = Synthetic construct
  source 1..2187: mol_type = other DNA
atggccggcttctgggtgggcaccgcccccctggtggccgccggcagaagaggcagatggcccccccagcagctgatgctgagcgccgccctgagaaccctgaagcacgtgctgtactacagcagacagtgcctgatggtgagcagaaacctgggcagcgtgggctacgaccccaacgagaagaccttcgacaagatcctggtggccaacagaggcgagatcgcctgcagagtgatcagaacctgcaagaagatgggcatcaagaccgtggccatccacagcgacgtggacgccagcagcgtgcacgtgaagatggccgacgaggccgtgtgcgtgggccccgcccccaccagcaagagctacctgaacatggacgccatcatggaggccatcaagaagaccagagcccaggccgtgcaccccggctacggcttcctgagcgagaacaaggagttcgccagatgcctggccgccgaggacgtggtgttcatcggccccgacacccacgccatccaggccatgggcgacaagatcgagagcaagctgctggccaagaaggccgaggtgaacaccatccccggcttcgacggcgtggtgaaggacgccgaggaggccgtgagaatcgccagagagatcggctaccccgtgatgatcaaggccagcgccggcggcggcggcaagggcatgagaatcgcctgggacgacgaggagaccagagacggcttcagactgagcagccaggaggccgccagcagcttcggcgacgacagactgctgatcgagaagttcatcgacaaccccagacacatcgagatccaggtgctgggcgacaagcacggcaacgccctgtggctgaacgagagagagtgcagcatccagagaagaaaccagaaggtggtggaggaggcccccagcatcttcctggacgccgagaccagaagagccatgggcgagcaggccgtggccctggccagagccgtgaagtacagcagcgccggcaccgtggagttcctggtggacagcaagaagaacttctacttcctggagatgaacaccagactgcaggtggagcaccccgtgaccgagtgcatcaccggcctggacctggtgcaggagatgatcagagtggccaagggctaccccctgagacacaagcaggccgacatcagaatcaacggctgggccgtggagtgcagagtgtacgccgaggacccctacaagagcttcggcctgcccagcatcggcagactgagccagtaccaggagcccctgcacctgcccggcgtgagagtggacagcggcatccagcccggcagcgacatcagcatctactacgaccccatgatcagcaagctgatcacctacggcagcgacagaaccgaggccctgaagagaatggccgacgccctggacaactacgtgatcagaggcgtgacccacaacatcgccctgctgagagaggtgatcatcaacagcagattcgtgaagggcgacatcagcaccaagttcctgagcgacgtgtaccccgacggcttcaagggccacatgctgaccaagagcgagaagaaccagctgctggccatcgccagcagcctgttcgtggccttccagctgagagcccagcacttccaggagaacagcagaatgcccgtgatcaagcccgacatcgccaactgggagctgagcgtgaagctgcacgacaaggtgcacaccgtggtggccagcaacaacggcagcgtgttcagcgtggaggtggacggcagcaagctgaacgtgaccagcacctggaacctggccagccccctgctgagcgtgagcgtggacggcacccagagaaccgtgcagtgcctgagcagagaggccggcggcaacatgagcatccagttcctgggcaccgtgtacaaggtgaacatcctgaccagactggccgccgagctgaacaagttcatgctggagaaggtgaccgaggacaccagcagcgtgctgagaagccccatgcccggcgtggtggtggccgtgagcgtgaagcccggcgacgccgtggccgagggccaggagatctgcgtgatcgaggccatgaagatgcagaacagcatgaccgccggcaagaccggcaccgtgaagagcgtgcactgccaggccggcgacaccgtgggcgagggcgacctgctggtggagctggagtga

SEQ ID NO: 7 - DNA, 2187 bp, synthetic construct
  misc_feature 1..2187: note = Synthetic construct
  source 1..2187: mol_type = other DNA
atggccggattttgggtcggaactgcaccacttgtcgctgccggtagaagaggaagatggccaccgcagcaactgatgttgagcgctgcactgcgcacactgaagcatgtgctgtactactcgcgccagtgtttgatggtgtccaggaatctcggctccgtgggctacgaccccaacgaaaagacttttgacaagatcctcgtggccaacagaggggaaattgcgtgccgcgtgattcggacttgcaagaagatgggaatcaagaccgtggccatacactccgatgtggacgcctcctccgtccacgtcaagatggctgatgaagccgtctgcgtgggaccggcgcctacttccaagtcgtaccttaacatggacgccatcatggaggccatcaagaaaaccagggcgcaggcggtgcatcctggctacggcttcctgtccgaaaacaaggagttcgcacggtgcctggccgccgaggacgtggtctttatcgggcccgacacccatgcaatccaggccatgggcgacaagatcgagtcgaagctgctggcgaagaaggcagaagtgaacactattcccgggttcgacggagtggtcaaagacgcggaagaggccgtccgaatcgcccgggagattggataccctgtgatgattaaggcctcggctggcggaggcggaaagggaatgcgcattgcctgggatgacgaagaaacccgggatggattccggctgagctcccaagaagccgcatcgtccttcggggacgatagactgctgatcgaaaagttcatcgacaacccaaggcacatcgaaatccaggtcctcggggacaagcatggaaacgccctgtggttgaacgagagagagtgctccattcaacggcgcaaccagaaggtcgtggaggaagccccctcgattttcctcgatgctgaaactcgccgggccatgggggagcaagcggtggccctggcccgcgcagtgaagtactcctcggccgggaccgtggagttcctggtggacagcaaaaagaacttctactttctcgagatgaacaccaggctccaagtggagcaccctgtgaccgaatgcatcactggacttgacctggtgcaggaaatgatccgcgtggccaagggataccccctgaggcacaagcaggccgacatcagaatcaacggttgggccgtggaatgtcgggtgtacgctgaggatccgtataagtccttcggcttgccgagcatcggacggctgtcacagtaccaggaacccctgcaccttcctggagtcagagtggactccggaatccaacctggttcggacatttccatctactacgatccgatgatctccaaactcattacctacggtagcgaccggaccgaggctctgaaacgcatggctgacgccctggacaactatgtcatccggggagtcactcacaatatcgcgctgctgcgcgaagtcatcattaatagccgcttcgtgaagggcgacatttccaccaagttcctgagcgacgtgtaccctgatggtttcaagggtcacatgctgactaagtccgagaagaaccagctcctcgctatcgcgtcctccctgtttgtggcgttccagctgagggcccagcacttccaagaaaactcaagaatgccggtcatcaagcccgacattgccaattgggaactgagcgtgaagctgcacgacaaagtgcacaccgtggtggccagcaacaacggctccgtgttctccgtggaagtggatgggtcaaagctgaacgtgaccagcacctggaacctggcgtccccgctcctgtcagtgtccgtggacggcactcagcggactgtgcagtgtttgtcccgggaagccgggggcaatatgagcatccagttcctcgggacggtgtacaaggtcaacatcctcactcggttggccgctgaactcaacaagttcatgctggaaaaggtcaccgaggacacctcctctgtgctgcggtcgcccatgccgggagtggtcgtggccgtgtccgtgaagcctggcgatgccgtggccgaaggtcaagaaatttgcgtgatcgaggccatgaagatgcagaactcgatgacggccggaaagaccggcaccgtcaaaagcgtgcactgccaggccggcgataccgtgggagagggcgatctgctcgtggaactcgaatga

SEQ ID NO: 8 - protein, 728 aa, Homo sapiens
MAGFWVGTAPLVAAGRRGRWPPQQLMLSAALRTLKHVLYYSRQCLMVSRNLGSVGYDPNEKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVYKVNILTRLAAELNKFMLEKVTEDTSSVLRSPMPGVVVAVSVKPGDAVAEGQEICVIEAMKMQNSMTAGKTGTVKSVHCQAGDTVGEGDLLVELE

SEQ ID NO: 9 - DNA, 7362 bp, synthetic construct
  misc_feature 1..7362: note = Synthetic construct
  source 1..7362: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggcggggttctgggtcgggacagcaccgctggtcgctgccggacggcgtgggcggtggccgccgcagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 10 - DNA, 7255 bp, synthetic construct
  misc_feature 1..7255: note = Synthetic construct
  source 1..7255: mol_type = other DNA
ccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtagccatgctctagcgatcgcggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtactgaattcgccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatcgaattccctgcaggtagagcatggctacgtaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 11 - DNA, 6533 bp, synthetic construct
  misc_feature 1..6533: note = Synthetic construct
  source 1..6533: mol_type = other DNA
gggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcttctagactgcgcgctcgctcgctcactgaggccgcccgggcaaagcccgggcgtcgggcgacctttggtcgcccggcctcagtgagcgagcgagcgcgcagagagggagtggccaactccatcactaggggttcctttaattaatacgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacgtaagcggccgcggcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgg

SEQ ID NO: 12 - DNA, 6530 bp, synthetic construct
  misc_feature 1..6530: note = Synthetic construct
  source 1..6530: mol_type = other DNA
gggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcttctagactgcgcgctcgctcgctcactgaggccgcccgggcaaagcccgggcgtcgggcgacctttggtcgcccggcctcagtgagcgagcgagcgcgcagagagggagtggccaactccatcactaggggttcctttaattaatacgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgagataacaggcctattgattggaaagtttgtcaacgaattgtgggtcttttggggtttgctgccccttttacgcaatgtggatatcctgctttattgcctttatatgcatgtatacaagcaaaacaggcttttactttctcgccaacttacaaggcctttctcagtaaacagtatagaccctttaccccgttgctcggcaacggcctggtctgtgccaagtgtttgctgacgcaacccccactggttggggcttggccataggccatcagcgcagcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccaggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacgtaagcggccgcggcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgg

SEQ ID NO: 13 - DNA, 189 bp, synthetic construct
  misc_feature 1..189: note = Synthetic construct
  source 1..189: mol_type = other DNA
cctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagag

SEQ ID NO: 14 - DNA, 318 bp, synthetic construct
  misc_feature 1..318: note = Synthetic construct
  source 1..318: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgaga

SEQ ID NO: 15 - DNA, 251 bp, synthetic construct
  misc_feature 1..251: note = Synthetic construct
  source 1..251: mol_type = other DNA
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtg

SEQ ID NO: 16 - DNA, 392 bp, synthetic construct
  misc_feature 1..392: note = Synthetic construct
  source 1..392: mol_type = other DNA
tgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 17 - DNA, 133 bp, synthetic construct
  misc_feature 1..133: note = Synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 18 - DNA, 447 bp, synthetic construct
  misc_feature 1..447: note = Synthetic construct
  source 1..447: mol_type = other DNA
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaaggaattc

SEQ ID NO: 19 - DNA, 165 bp, synthetic construct
  misc_feature 1..165: note = Synthetic construct
  source 1..165: mol_type = other DNA
gtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcag

SEQ ID NO: 20 - DNA, 189 bp, synthetic construct; identical to SEQ ID NO: 13
  misc_feature 1..189: note = Synthetic construct
  source 1..189: mol_type = other DNA

SEQ ID NO: 21 - DNA, 710 bp, synthetic construct
  misc_feature 1..710: note = Synthetic construct
  source 1..710: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 22 - DNA, 1157 bp, synthetic construct
  misc_feature 1..1157: note = Synthetic construct
  source 1..1157: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaaggaattc

SEQ ID NO: 23 - DNA, 100 bp, synthetic construct
  misc_feature 1..100: note = Synthetic construct
  source 1..100: mol_type = other DNA
aggttaatttttaaaaagcagtcaaaagtccaagtggcccttggcagcatttactctctctgtttgctctggttaataatctcaggagcacaaacattcc

SEQ ID NO: 24 - DNA, 206 bp, synthetic construct
  misc_feature 1..206: note = Synthetic construct
  source 1..206: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattcc

SEQ ID NO: 25 - DNA, 460 bp, synthetic construct
  misc_feature 1..460: note = Synthetic construct
  source 1..460: mol_type = other DNA
gggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 26 - DNA, 666 bp, synthetic construct
  misc_feature 1..666: note = Synthetic construct
  source 1..666: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccgggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 27 - DNA, 588 bp, synthetic construct
  misc_feature 1..588: note = Synthetic construct
  source 1..588: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 28 - DNA, 63 bp, synthetic construct
  misc_feature 1..63: note = Synthetic construct
  source 1..63: mol_type = other DNA
gggagtatattagtgctaatttccctccgtttgtcctagcttttctcttctgtcaaccccaca

SEQ ID NO: 29 - DNA, 81 bp, synthetic construct
  misc_feature 1..81: note = Synthetic construct
  source 1..81: mol_type = other DNA
gggattcatgaaaatccactactccagacagacggctttggaatccaccagctacatccagctccctgagcagagttgaga

SEQ ID NO: 30 - DNA, 264 bp, synthetic construct
  misc_feature 1..264: note = Synthetic construct
  source 1..264: mol_type = other DNA
gggacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaca

SEQ ID NO: 31 - DNA, 598 bp, synthetic construct
  misc_feature 1..598: note = Synthetic construct
  source 1..598: mol_type = other DNA
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 32 - DNA, 726 bp, synthetic construct
  misc_feature 1..726: note = Synthetic construct
  source 1..726: mol_type = other DNA
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 33 - DNA, 313 bp, synthetic construct
  misc_feature 1..313: note = Synthetic construct
  source 1..313: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 34 - DNA, 235 bp, synthetic construct
  misc_feature 1..235: note = Synthetic construct
  source 1..235: mol_type = other DNA
tttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 35 - DNA, 6607 bp, synthetic construct
  misc_feature 1..6607: note = Synthetic construct
  source 1..6607: mol_type = other DNA
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcagccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattag

SEQ ID NO: 36 - DNA, 6047 bp, synthetic construct
  misc_feature 1..6047: note = Synthetic construct
  source 1..6047: mol_type = other DNA
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcgcgccaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgtcgagaattcagccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattag

SEQ ID NO: 37 - DNA, 4657 bp, synthetic construct
  misc_feature 1..4657: note = Synthetic construct
  source 1..4657: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagctagtaacggccgccagtgtgctggaattcggcttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagcttgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggccctgcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgacatcacatttaaaagcatctcaggtaactatattttgaatttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttctgtacacgcgatccatcacactggcggccgctcgactggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 4791 bp, synthetic construct
  misc_feature 1..4791: note = Synthetic construct
  source 1..4791: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtagccatgctctagcgatcgcggtaccaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacagccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggattccaggggtgtgtttcgtcgagatgcacgtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccagacaagagtgaggttgctcatcggtttaaagatttgggagaagaaaatttcaaagccttgtaagttaaaatattgatgaatcaaatttaatgtttctaatagtgttgtttattattctaaagtgcttatatttccttgtcatcagggttcagattctaaaacagtgctgcctcgtagagttttctgcgttgaggaagatattctgtatctgggctatccaataaggtagtcactggtcacatggctattgagtacttcaaatatgacaagtgcaactgagaaacaaaaacagcaattcgttgatcgaattccctgcaggtagagcatggctacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 39 - DNA, 9008 bp, synthetic construct
  misc_feature 1..9008: note = Synthetic construct
  source 1..9008: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcaagtttgtacaaaaaagcaggctgccaccatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 40 - DNA, 9477 bp, synthetic construct
  misc_feature 1..9477: note = Synthetic construct
  source 1..9477: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 41 - DNA, 4024 bp, synthetic construct
  misc_feature 1..4024: note = synthetic
  source 1..4024: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 42 - DNA, 6435 bp, synthetic construct
  source 1..6435: mol_type = other DNA
atgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcac

SEQ ID NO: 43 - DNA, 2112 bp, synthetic construct
  source 1..2112: mol_type = other DNA
atgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtga

SEQ ID NO: 44 - DNA, 6431 bp, synthetic construct
  source 1..6431: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagacggtcagactaaactggctgacggaatttatgcctcttccgaccatcaagcattttatccgtactcctgatgatgcatggttactcaccactgcgatccctggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcac

(19) United States
(12) Patent Application Publication
(10) Pub. No.: US 2025/0049952 A1
(43) Pub. Date: Feb. 13, 2025

(54) IMPROVED GENE THERAPY CONSTRUCTS FOR THE TREATMENT OF PROPIONIC ACIDEMIA CAUSED BY MUTATIONS IN PROPIONYL-COA CARBOXYLASE ALPHA

(71) Applicant: The United States of America,as represented by the Secretary,Department of Health and Human Services, Bethesda, MD (US)

(72) Inventors: Charles P. Venditti, Potomac, MD (US); Randy J. Chandler, Chevy Chase, MD (US)

(73) Assignee: The United States of America,as represented by the Secretary,Department of Health and Human Services, Bethesda, MD (US)

(21) Appl. No.: 18/723,020

(22) PCT Filed: Dec. 29, 2022
(86) PCT No.: PCT/US2022/082527

§ 371 Date: Jun. 21, 2024

Related U.S. Application Data
(60) Provisional application No. 63/294,511, filed on Dec. 29, 2021.

(51) Int. Cl. A61K 48/00; A61P 3/00; C12N 9/00; C12N 9/22; C12N 15/11; C12N 15/86; G01N 33/50

(52) U.S. Cl. CPC A61K 48/005; A61K 48/0083; A61P 3/00; C12N 9/22; C12N 9/93; C12N 15/111; C12N 15/86; C12Y 604/01003; G01N 33/5023; C12N 2310/20; C12N 2750/14143; C12N 2830/48; C12N 2830/50; G01N 2333/9015; G01N 2800/52

(57) ABSTRACT

Polynucleotide expression cassettes comprising synthetic polynucleotides encoding human propionyl-CoA carboxylase alpha (synPCCA) are described herein. Related recombinant expression vectors, recombinant adeno-associated viruses (rAAVs), and compositions are also described. Also described are methods of treating a disease or condition mediated by propionyl-CoA carboxylase, comprising administering to a subject in need thereof a therapeutic amount of any of the polynucleotide expression cassettes, recombinant expression vectors, rAAVs, or compositions.

### CROSS-REFERENCE TO RELATED APPLICATION

This patent application claims the benefit of U.S. Provisional Patent Application No. 63/294,511, filed Dec. 29, 2021, which is incorporated by reference in its entirety herein.

### STATEMENT REGARDING FEDERALLY SPONSORED RESEARCH OR DEVELOPMENT

This invention was made with Government support under project number Z01HG200318-14 by the National Institutes of Health, National Human Genome Research Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 165,079 Byte XML file named “766164.xml,” dated Dec. 29, 2022.

### BACKGROUND OF THE INVENTION

Propionic acidemia (PA) is a life-threatening autosomal recessive disorder of organic acid metabolism in humans with an estimated incidence of 1:250,000-1:750,000 births. It is caused by a deficiency of propionyl-CoA carboxylase (PCC), a ubiquitously expressed, heteropolymeric mitochondrial enzyme involved primarily in the catabolism of propiogenic amino acids, particularly isoleucine, valine, methionine, and threonine, as well as odd-chain fatty acids.

Most frequently, PA presents in the neonatal period with hyperammonemia, vomiting, poor feeding, and hypotonia and progresses into a life-threatening metabolic crisis. Patients who survive suffer from recurrent metabolic instability and can develop multisystemic complications, especially cardiomyopathy.

Over the decades, it has been repeatedly documented that PA patients with an early and severe clinical course experience increased mortality and disease associated morbidity (Surtees et al., Pediatr. Neurol., 8(5): 333-7 (1992); Shchelochkov et al., Propionic Acidemia, May 17, 2012 [updated Oct. 6, 2016]. In: GeneReviews [Internet]. Seattle (WA): University of Washington, Seattle; 1993-2021. PMID: 22593918).

The recalcitrant nature of the disorder to conventional medical management, including the dietary restriction of amino acid precursors, L-carnitine supplementation, and administration of metronidazole to reduce the generation of propionic acid by intestinal bacteria, has led to the implementation of elective liver transplantation (LT) as an experimental surgical treatment for PA.

While not curative of all aspects of the disorder, successful LT in the setting of PA provides restoration of metabolic stability and protection from early death, and therefore represents a clinical benchmark for gene replacement or addition approaches that might increase hepatic PCC expression and activity. Nevertheless, organ availability, surgical complications, and toxic side effects of life-long immunosuppression after LT, including the development of the post transplant lymphoproliferative disorder (PTLD), remain as practical constraints to the widespread adoption of LT as a preferred treatment for PA.

Accordingly, there exists a need for improved treatments for PA.

### BRIEF SUMMARY OF THE INVENTION

An aspect of the invention provides a polynucleotide expression cassette comprising: (a) a synthetic PCCA polynucleotide (synPCCA) selected from the group consisting of: (i) a polynucleotide comprising the nucleic acid sequence of any one of SEQ ID NOs: 2-7; and (ii) a polynucleotide comprising a nucleic acid sequence with at least 80% identity to the nucleic acid sequence of any one of SEQ ID NOs: 2-7 which encodes the polypeptide of SEQ ID NO:8 and has equivalent or greater expression in a host relative to expression of any one of SEQ ID NOs: 2-7 or SEQ ID NO:1, wherein the polynucleotide does not have the nucleic acid sequence of SEQ ID NO:1; and (b) any one or more of the following features: (i) a 3′ inverted terminal repeat 3′ ITR with a length greater than 135 base pairs (bp) and less than 145 bp; (ii) fewer CpG dinucleotides as compared to a nucleic acid sequence of any one or more of SEQ ID NOs: 9-12 and 35-40; and (iii) fewer restriction enzyme recognition sites as compared to a nucleic acid sequence of any one or more of SEQ ID NOs: 9-12 and 35-40.

Further aspects of the invention provide recombinant expression vectors, recombinant adeno-associated viruses (rAAVs), and compositions related to the inventive polynucleotide expression cassettes.

Another aspect of the invention provides methods of treating a disease or condition mediated by PCC, comprising administering to a subject in need thereof a therapeutic amount of any of the inventive polynucleotide expression cassettes, recombinant expression vectors, rAAVs, or compositions described herein.

Another aspect of the invention provides methods of treating a disease or condition mediated by PCC in a subject in need thereof, comprising administering to a cell of the subject, or a population of cells of the subject, any of the inventive polynucleotide expression cassettes, recombinant expression vectors, rAAVs, or compositions described herein, wherein the polynucleotide expression cassette is inserted into the cell of the subject, or the population of cells of the subject, via genome editing on the cell of the subject, or the population of cells of the subject, using a nuclease selected from the group of zinc finger nucleases (ZFNs), transcription activator-like effector nucleases (TALENs), the clustered regularly interspaced short palindromic repeats (CRISPR/cas system) and meganuclease re-engineered homing endonucleases on the cell from the subject, or the population of cells of the subject; and administering the cell, or population of cells, to the subject.

Still another aspect of the invention provides a nucleic acid consisting of (a) a nucleotide sequence that is at least 12 but no more than 35 contiguous nucleotides of any one of SEQ ID NO: 2-7; (b) a nucleotide sequence that is at least 90% identical to at least 12 but no more than 35 contiguous nucleotides of any one of SEQ ID NO: 2-7; or (c) a nucleotide sequence that is complementary to (a) or (b).

Another aspect of the invention provides an in vitro method for detecting the expression of synPCCA nucleic acid by cells, the method comprising: (a) contacting a sample comprising nucleic acid from the cells with any of the inventive nucleic acids or collections of nucleic acids described herein, thereby forming a complex; and (b) detecting the complex, wherein detection of the complex is indicative of the expression of synPCCA nucleic acid by the cells.

### BRIEF DESCRIPTION OF THE SEVERAL VIEWS OF THE DRAWINGS

FIG. 1A presents the ClustalW weighted sequence distances and percent sequence identity of different PCCA alleles versus wild type PCCA, and each other, showing that all the synPCCA sequences (SEQ ID NOs: 2-7) differ from the wild type PCCA gene (SEQ ID NO: 1) by >20% at the nucleotide level, and similarly, diverge from each other between 11-24%.

FIG. 1B shows the characterization of distinct feature of the synPCCA sequences (SEQ ID NOs: 2-7) and the wild type PCCA gene (SEQ ID NO: 1) using a phylogenetic analysis where distinct grouping is apparent.

FIG. 2 presents a western blot showing PCCA protein expression in 293 cells, which are human transformed kidney cells, transfected with AAV backbones expressing GFP or either wild-type or synPCCA under the control of various promoter/enhancer combinations. PCCA=propionyl-CoA carboxylase alpha subunit, CBA=chicken beta actin, EF1a=elongation factor 1 alpha, EF1aS=elongation factor 1 alpha short.

FIG. 3 presents synPCCA directed PCCA protein levels relative to wild-type PCCA expression in transfected 293 cells, quantified from the western blot in FIG. 2. The PCCA expression is much higher in 293 cells transfected with CBA-synPCCA1 versus those transfected with CBA-PCCA (wild-type). The levels of CBA-PCCA (wild-type) are comparable to the expression achieved when using a weaker promoter and distinct synPCCA6 allele, EF1a-synPCCA6.

FIG. 4 Survival in untreated Pcca^{-/-} (n=12) mice compared to Pcca^{-/-} mice (n=4) treated with 3×10¹¹ VC of AAV-CBA-synPCCA1 delivered by intrahepatic injection at birth. Treated Pcca^{-/-} mice display a significant increase in survival and were indistinguishable from their wild-type littermates on day 30 of life when they were sacrificed for further study.

FIG. 5 shows plasma methylcitrate levels in untreated Pcca^{-/-} (n=6) mice and Pcca^{-/-} mice (n=6) treated with 3×10¹¹ VC of AAV-CBA-synPCCA1 by systemic injection at birth. Treated Pcca^{-/-} mice have a significant decrease in the disease related biomarker, 2-methylcitrate.

FIG. 6 shows Western blots of murine livers, from wild-type mice (Pcca^{+/+} and Pcca^{-/-}), an untreated PA mouse (Pcca^{-/-}), and Pcca^{-/-} mouse treated with 3×10¹¹ VC of AAV9-CBA-synPCCA1. The AAV treated mouse was sacrificed on day of life 30 and injected on day of life 1. The treated Pcca^{-/-} mouse displays hepatic PCCA expression whereas the untreated Pcca^{-/-} mice shows no hepatic murine PCCA expression. The antibody used for western blot can detect both human (PCCA) and murine (Pcca). Ubiquinol-Cytochrome C Reductase Core protein 2 (Uqcrc2) was used as a loading control.

FIG. 7 shows transgene hepatic PCCA protein expression relative to wild-type murine PCCA expression in untreated and the AAV9 treated Pcca^{-/-} mouse quantified from Western blot in FIG. 6.

FIG. 8 shows survival in an additional cohort of untreated Pcca^{-/-} (n=10) mice compared to Pcca^{-/-} mice (n=9) treated with 3×10¹¹ VC of AAV-CBA-synPCCA1 delivered by systemic injection at birth. Treated Pcca^{-/-} mice display a significant increase in survival and some treated mice were indistinguishable from their wild-type litter mates at day 30 and demonstrated long term survival to >150 days.

FIG. 9A shows a vector comprised of 145 base pair AAV2 inverted terminal repeats (5′ITR_{L} and 3′ ITR_{L}), the long elongation factor 1α promoter (EF1AL), an intron (I), the synPCCA1 gene, the rabbit beta-globin polyadenylation signal (rBGA). The production plasmid expresses the kanamycin resistance gene. FIG. 9B shows a vector comprised of 130 base pair AAV2 inverted terminal repeats (5′ITR_{S} and 3′ ITR_{S}), the short elongation factor 1α promoter (EF1AS), an intron (I), synPCCA1 gene, the hepatitis B post translation response element (HPRE), and the bovine growth hormone polyadenylation signal (BGHA). The production plasmid expresses the kanamycin resistance gene.

FIG. 10 presents a Western blot showing PCCA protein expression in 293 cells, which are human transformed kidney cells, after transfection with transfected with AAV backbones expressing synPCCA1 under the control of various promoter/enhancer combinations. PCCA=propionyl-CoA carboxylase alpha subunit, CBA=chicken beta actin, EF1a=elongation factor 1 alpha, EF1aS=elongation factor 1 alpha short. HPRE− hepatitis B post translation response element. HPREm− hepatitis B post translation response element, mutant. Beta-actin is the loading control. The fold change of protein expression compared to the basal level in 293T cells in indicated above as fold change.

FIG. 11 depicts survival in untreated Pcca^{-/-} (n=12) mice compared to Pcca^{-/-} mice (n=9) treated with 1×10¹¹ VC of AAV9-EF1aL-synPCCA1 (n=18), 1×10¹¹ VC of AAV9-EF1aS-synPCCA1-HPRE (n=15), or 4×10¹¹ VC of AAV9-EF1aS-synPCCA1-HPRE (n=5) delivered by retroorbital injection at birth. The treated Pcca^{-/-} mice display a significant increase in survival.

FIG. 12 shows the list of codon frequencies in the human proteome.

FIG. 13 is a schematic showing the pathway of human propionyl-CoA metabolism. PCC is a ubiquitously expressed, heteropolymeric mitochondrial enzyme involved primarily in the catabolism of propiogenic amino acids, particularly isoleucine, valine, methionine, and threonine, as well as odd-chain fatty acids. The enzyme is composed of α- and β-subunits encoded by their respective genes, PCCA and PCCB, and PA is caused by pathogenic variants in either gene. PCC catalyzes the first step in the conversion of propionyl-CoA to D-methylmalonyl-CoA in the pathway of propionyl-CoA oxidation. The formation of 2-MC, a biomarker generated through the condensation of oxaloacetic acid (OAA) and propionyl-CoA is also noted, as are downstream enzymatic steps in the pathway, including D-methylmalonyl-CoA epimerase (MCEE) and methylmalonyl-CoA mutase (MMUT), in the metabolism of propionyl-CoA into the citric acid (Krebs) cycle.

FIG. 14 is a graph showing the plasma 2-methylcitrate levels of the mice models of Table 3 and in WT mice.

FIG. 15A is a schematic comparing the general structures of the parent vector transgene plasmid (AAV9-Ef1S-synPCCA1-HPRE parent) (top) and the improved vector transgene plasmid (AAV9-Ef1S-synPCCA1-HPRE final) (bottom). ITR—inverted terminal repeat. EF1S—elongation factor 1 promoter, short. Intron—synthetic intron. hPCCA1—synPCCA1, a codon optimized PCCA gene. HPRE—the hepatitis B derived post-translational response element. BGH A—bovine growth hormone polyadenylation signal. ABX—antibiotic, kanamycin resistance. Att1 and AttL2—bacterial attachment site L1 and L2.

FIG. 15B is a schematic comparing the general structures of the improved vector transgene plasmid (AAV9-Ef1S-synPCCA1-HPRE final). Abbreviations are as described for FIG. 15A.

FIGS. 16A-16B are images showing the results of Western blot analyses of expression of PCCA encoded by the AAV9.CB7.C1.hPCCA1.RBG vector (16A) and AAV9-Ef1S-synPCCA1-HPRE final (16B) by HEPG2 cells.

FIG. 16C is a graph showing the amount of PCCA expressed (% of wild-type (WT)) by the AAV9.CB7.C1.hPCCA1.RBG vector or AAV9-Ef1S-synPCCA1-HPRE Final after infection of HEPG2 PCCA knock-out cells. 25 pg protein/lane. PCCA (rabbit polyclonal) Ab: abcam, cat. #ab187686 (1:1000).

FIG. 17 is a graph showing the percentage of mice surviving at the indicated number of days post-treatment with the indicated doses of AAV9-Ef1S-synPCCA1-HPRE final at P0-1. No treatment (untreated) was used as a control. The graph depicts the percent survival of different cohorts of animals **** P<0.0001, ** P<0.01 compared to survival of untreated vs treated Pcca^{-/-} mice. P values were calculated using a Log_rank (Mantel-Cox) test.

FIG. 18 is a graph showing the weight gain (grams) at the indicated number of days post-treatment with the indicated doses of AAV9-Ef1S-synPCCA1-HPRE Final in the various treated Pcca^{-/-} mice compared to treated Pcca^{+/-} control littermates. Mice were treated with the indicated doses of AAV9-Ef1S-synPCCA1-HPRE Final at P0-1.

FIG. 19A is a graph showing methylcitrate levels in response to treatment with AAV9-Ef1S-synPCCA1-HPRE Final on day of life (DOL) 30 (Dose 1e11 GC/pup). The untreated controls were obtained on DOL1 due to the lethal phenotype of the Pcca^{-/-} genotype. Untreated Pcca^{-/-} do not survive long enough to be studied.

FIG. 19B is a graph showing 1-C-13 propionate oxidation (cumulative percentage of dose metabolized) compared to untreated heterozygous controls as measured on DOL 30 in response to treatment with AAV9-Ef1S-synPCCA1-HPRE Final (Dose 1e11 GC/pup) and the indicated number of minutes after treatment. The untreated controls were not obtainable due to the lethal phenotype of the Pcca^{-/-} genotype on DOL1. The treated Pcca^{-/-} mutant mice have nearly normal oxidative capacity. Untreated Pcca^{-/-} do not survive long enough to be studied.

FIG. 20A is a Western blot showing PCCA protein expression in the liver or hearts of mice on DOL 30 in response to treatment with AAV9-Ef1S-synPCCA1-HPRE Final (Dose 1e11 GC/pup). In the liver, the control band is Actin and in the heart it is GAPDH.

FIG. 20B is a graph showing PCCA biodistribution after treatment with AAV9-Ef1S-synPCCA1-HPRE Final. FIG. 20B shows the vector copy numbers in the respective tissues at the varying doses used.

FIG. 20C is a graph showing PCCA expression in the liver after treatment with AAV9-Ef1S-synPCCA1-HPRE final on DOL 30 (Dose 1e11 GC/pup). FIG. 20C shows the results of RT-qPCR to detect the synPCCA transcript (SynPcca) compared the endogenous mouse Pcca gene (MmPcca) relative to actin. WT=Pcca^{+/+}; HT=Pcca^{+/-}; MT=Pcca^{-/-}; MT+AAV9=Pcca^{-/-} treated with AAV9-Ef1S-synPCCA1-HPRE Final. N=3 mice per group with a dose of 1e11 GC/pup.

FIG. 20D is a graph showing PCCA expression in the heart after treatment with AAV9-Ef1S-synPCCA1-HPRE Final on DOL 30 (Dose 1e11 GC/pup). FIG. 20D shows the results of RT-qPCR to detect the synPCCA transcript (SynPcca) compared the endogenous mouse Pcca gene (MmPcca) relative to Gapdh. WT=Pcca^{+/+}; HT=Pcca^{+/-}; MT=Pcca^{-/-}; MT+AAV9=Pcca^{-/-} treated with AAV9-Ef1S-synPCCA1-HPRE final. N=3 mice per group with a dose of 1e11 GC/pup.

FIG. 20E are images showing the results of an RNA in situ hybridization study to detect the expression of synPCCA in Pcca^{-/-} mice after treatment with AAV9-Ef1S-synPCCA1-HPRE Final on DOL 30 (Doses: 1e10 GC/pup, 1e11 GC/pup, 4e11 GC/pup). Liver and heart tissues were removed, fixed in formalin and stained with an RNA probe designed to specifically hybridize to the synPCCA mRNA. The dark staining cells in the liver are hepatocytes or cardiomyocytes in the heart, and increase in number with a higher dose of AAV.

### DETAILED DESCRIPTION OF THE INVENTION

Polynucleotide expression cassettes have been designed which provide any one or more of a variety of advantages over other recombinant expression vectors comprising a synthetic propionyl-CoA carboxylase subunit a (PCCA) polynucleotide (synPCCA), for example, those described in U.S. patent application Ser. No. 17/620,331, which is incorporated herein by reference in its entirety. For example, the inventive polynucleotide expression cassettes may provide any one or more of reduced immunogenicity, improved packaging, improved stability, and higher titer yield, as compared to other recombinant expression vectors comprising synPCCA, for example, those described in U.S. patent application Ser. No. 17/620,331 (e.g., a nucleic acid sequence of any one or more of SEQ ID NOs: 9-12 and 35-40). For example, in the field of molecular cloning, modifications to expression cassettes often may, disadvantageously, reduce the titer yield. In contrast, the inventive polynucleotide expression cassettes may provide a high titer yield, which is not always achieved with polynucleotide expression cassettes in the field of molecular cloning. The inventive polynucleotide expression cassettes may also provide any one or both of fewer CpG dinucleotides and fewer restriction enzyme recognition sites as compared to other recombinant expression vectors comprising synPCCA, for example, those described in U.S. patent application Ser. No. 17/620,331 (e.g., a nucleic acid sequence of any one or more of SEQ ID NOs: 9-12 and 35-40). Because CpG dinucleotides can potentially stimulate an undesirable immune response in a patient, the inventive polynucleotide expression cassettes may provide reduced immunogenicity as compared to other recombinant expression vectors comprising synPCCA, for example, those described in U.S. patent application Ser. No. 17/620,331 (e.g., a nucleic acid sequence of any one or more of SEQ ID NOs: 9-12 and 35-40).

Accordingly, an aspect of the invention provides a polynucleotide expression cassette comprising: (a) a synPCCA polynucleotide and (b) any one or more of the following features: (i) a 3′ inverted terminal repeat (3′ ITR) with a length greater than 135 base pairs (bp) and less than 145 bp; (ii) fewer CpG dinucleotides as compared to a nucleic acid sequence of any one or more of SEQ ID NOs: 9-12 and 35-40; and (iii) fewer restriction enzyme recognition sites as compared to a nucleic acid sequence of any one or more of SEQ ID NOs: 9-12 and 35-40.

In an aspect of the invention, the 3′ ITR has a length that is greater than 135 bp and less than 145 bp, greater than 136 bp and less than 144 bp, greater than 137 bp and less than 143 bp, or greater than 138 bp and less than 142 bp. In a preferred aspect of the invention, the 3′ ITR has a length of 141 bp.

The 3′ ITR and the 5′ ITR of the polynucleotide expression cassette may be heterologous. In an aspect of the invention, the polynucleotide expression cassette comprises a 5′ inverted terminal repeat (5′ ITR) with a length greater than 125 base pairs (bp) and less than 135 bp. In an aspect of the invention, the 5′ ITR has a length that is greater than 126 bp and less than 134 bp, greater than 127 bp and less than 133 bp, greater than 128 bp and less than 132 bp, or greater than 129 bp and less than 131 bp. In a preferred aspect of the invention, the 5′ ITR has a length of 130 bp.

In an aspect of the invention, the polynucleotide expression cassette has fewer CpG dinucleotides as compared to other recombinant expression vectors comprising synPCCA, for example, a nucleic acid sequence of any one or more of SEQ ID NOs: 9-12 and 35-40. In a CpG dinucleotide, cytidine is joined by a 5′ to 3′ phospho-diester linkage to guanidine as part of a DNA strand. In an aspect of the invention, the polynucleotide expression cassette has fewer CpG dinucleotides near the 3′ ITR, the 5′ ITR, or both the 3′ ITR and the 5′ ITR, as compared to other recombinant expression vectors comprising synPCCA, such as those described in the foregoing paragraph.

For example, the inventive polynucleotide expression cassette may comprise 1% fewer, 2% fewer, 3% fewer, 4% fewer, 5% fewer, 6% fewer, 7% fewer, 8% fewer, 9% fewer, 10% fewer, 11% fewer, 12% fewer, 13% fewer, 14% fewer, 15% fewer, 16% fewer, 17% fewer, 18% fewer, 19% fewer, 20% fewer, 21% fewer, 22% fewer, 23% fewer, 24% fewer, 25% fewer, 26% fewer, 27% fewer, 28% fewer, 29% fewer, 30% fewer, 31% fewer, 32% fewer, 33% fewer, 34% fewer, 35% fewer, 36% fewer, 37% fewer, 38% fewer, 39% fewer, 40% fewer, 41% fewer, 42% fewer, 43% fewer, 44% fewer, 45% fewer, 46% fewer, 47% fewer, 48% fewer, 49% fewer, 50% fewer, 51% fewer, 52% fewer, 53% fewer, 54% fewer, 55% fewer, 56% fewer, 57% fewer, 58% fewer, 59% fewer, 60% fewer, 61% fewer, 62% fewer, 63% fewer, 64% fewer, 65% fewer, 66% fewer, 67% fewer, 68% fewer, 69% fewer, 70% fewer, 71% fewer, 72% fewer, 73% fewer, 74% fewer, 75% fewer, 76% fewer, 77% fewer, 78% fewer, 79% fewer, 80% fewer, 81% fewer, 82% fewer, 83% fewer, 84% fewer, 85% fewer, 86% fewer, 87% fewer, 88% fewer, 89% fewer, 90% fewer, 91% fewer, 92% fewer, 93% fewer, 94% fewer, 95% fewer, 96% fewer, 97% fewer, 98% fewer, or 99% fewer CpG dinucleotides as compared to other recombinant expression vectors comprising synPCCA, for example, a nucleic acid sequence of any one or more of SEQ ID NOs: 9-12 and 35-40In an aspect, the inventive polynucleotide expression cassette may comprise no CpG dinucleotides.

In an aspect of the invention, the polynucleotide expression cassette has fewer restriction enzyme recognition sites as compared to other recombinant expression vectors comprising synPCCA, for example, a nucleic acid sequence of any one or more of SEQ ID NOs: 9-12 and 35-40. Restriction enzyme recognition sites are located on a polynucleotide molecules, e.g., a DNA molecule, and contain specific sequences of nucleotides (e.g., 4 to 8 base pairs in length), which are recognized by restriction enzymes. Restriction enzyme recognition sites are well-known in the art. An example of a restriction enzyme recognition site, the occurrences of which are reduced or eliminated in the inventive polynucleotide expression cassettes, is an attachment L (AttL) site.

For example, the inventive polynucleotide expression cassette may comprise 1% fewer, 2% fewer, 3% fewer, 4% fewer, 5% fewer, 6% fewer, 7% fewer, 8% fewer, 9% fewer, 10% fewer, 11% fewer, 12% fewer, 13% fewer, 14% fewer, 15% fewer, 16% fewer, 17% fewer, 18% fewer, 19% fewer, 20% fewer, 21% fewer, 22% fewer, 23% fewer, 24% fewer, 25% fewer, 26% fewer, 27% fewer, 28% fewer, 29% fewer, 30% fewer, 31% fewer, 32% fewer, 33% fewer, 34% fewer, 35% fewer, 36% fewer, 37% fewer, 38% fewer, 39% fewer, 40% fewer, 41% fewer, 42% fewer, 43% fewer, 44% fewer, 45% fewer, 46% fewer, 47% fewer, 48% fewer, 49% fewer, 50% fewer, 51% fewer, 52% fewer, 53% fewer, 54% fewer, 55% fewer, 56% fewer, 57% fewer, 58% fewer, 59% fewer, 60% fewer, 61% fewer, 62% fewer, 63% fewer, 64% fewer, 65% fewer, 66% fewer, 67% fewer, 68% fewer, 69% fewer, 70% fewer, 71% fewer, 72% fewer, 73% fewer, 74% fewer, 75% fewer, 76% fewer, 77% fewer, 78% fewer, 79% fewer, 80% fewer, 81% fewer, 82% fewer, 83% fewer, 84% fewer, 85% fewer, 86% fewer, 87% fewer, 88% fewer, 89% fewer, 90% fewer, 91% fewer, 92% fewer, 93% fewer, 94% fewer, 95% fewer, 96% fewer, 97% fewer, 98% fewer, or 99% fewer restriction enzyme recognition sites as compared to other recombinant expression vectors comprising synPCCA, for example, a nucleic acid sequence of any one or more of SEQ ID NOs: 9-12 and 35-40.

The synPCCA polynucleotide may be as described, for example, in U.S. patent application Ser. No. 17/620,331. In the context of synPCCA, the terms “gene” and “transgene” are used interchangeably. A “transgene” is a gene that has been transferred from one organism to another. The polynucleotide sequences encoding the alpha subunit of PCC, synPCCA, allow for increased expression of the synPCCA gene relative to naturally occurring human PCCA sequences. These polynucleotide sequences are designed to not alter the naturally occurring human PCC alpha subunit amino acid sequence. They are also engineered or optimized to have increased transcriptional, translational, and protein refolding efficacy. This engineering is accomplished by using human codon biases, evaluating GC, CpG, and negative GpC content, optimizing the interaction between the codon and anti-codon, and eliminating cryptic splicing sites and RNA instability motifs. The synPCCA polynucleotides may also facilitate detection using nucleic acid-based assays.

As used herein, “PCCA” refers to the alpha subunit of human propionyl-CoA carboxylase, and “Pcca” refers to the alpha subunit of mouse propionyl-CoA carboxylase. PCC catalyzes the carboxylation of propionyl-CoA to D-methylmalonyl-CoA which is a metabolic precursor to succinyl-CoA, a component of the citric acid cycle or tricarboxylic acid cycle (TCA). The genes encoding the alpha and beta subunits of naturally occurring human propionyl-CoA carboxylase gene are referred to as PCCA or PCCB, respectively. The synthetic polynucleotide encoding the alpha subunit of PCC is referred to as synPCCA.

Naturally occurring human propionyl-CoA carboxylase is referred to as PCC, while synthetic PCC is designated as synPCC, even though the two are identical at the amino acid level.

“Codon optimization” refers to the process of altering a naturally occurring polynucleotide sequence to enhance expression in the target organism, e.g., humans. In the subject application, the human PCCA gene has been altered to replace codons that occur less frequently in human genes with those that occur more frequently and/or with codons that are frequently found in highly expressed human genes, see FIG. 11.

Codon optimization was employed to create six highly active and synthetic PCCA alleles designated PCCA1-6. This method involves determining the relative frequency of a codon in the protein-encoding genes in the human genome. For example, isoleucine can be encoded by AUU, AUC, or AUA, but in the human genome, AUC (47%), AUU (36%), and AUA (17%) are variably used to encode isoleucine in proteins. Therefore, in the proper sequence context, AUA would be changed to AUC to allow this codon to be more efficiently translated in human cells. FIG. 11 presents the codon usage statistics for a large fraction of human protein-encoding genes and serves as the basis for changing the codons throughout the PCCA cDNA.

Thus, the polynucleotide expression cassette comprises synthetic polynucleotides encoding PCCA selected from the group consisting of SEQ ID NOs: 2-7 and a polynucleotide sequence having at least 80% identity thereto. For those polynucleotides having at least 80% identity to SEQ ID NOs: 2-7, in additional aspects, they have at least 85%, at least 90%, at least 91%, at least 92%, at least 93%, at least 94%, at least 95%, at least 98%, or at least 99% identity to SEQ ID NOs: 2-7.

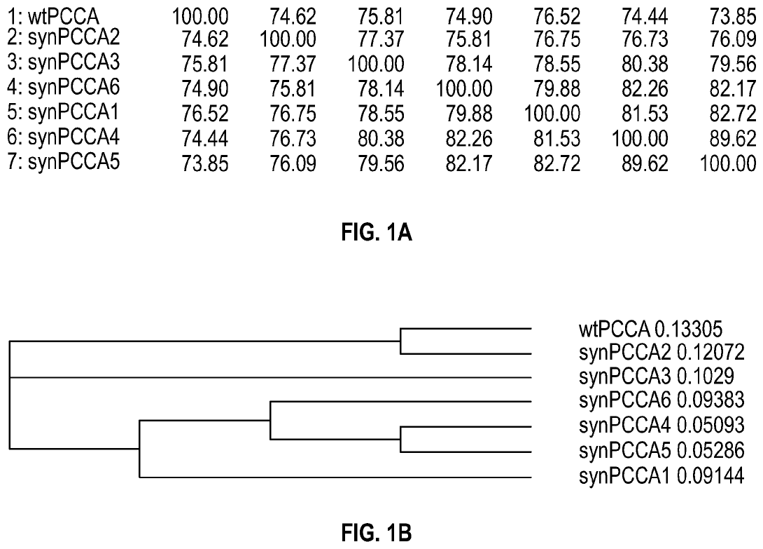

In one aspect, the subject synthetic polynucleotide encodes a polypeptide with 100% identity to the naturally occurring human PCC protein. FIG. 1A presents the ClustalW weighted sequence distances and percent sequence identity of different PCCA alleles versus wild type PCCA, and each other, showing that all the synPCCA sequences (SEQ ID NOs: 2-7) differ from the wild type PCCA gene (SEQ ID NO: 1) by >20% at the nucleotide level, and similarly, diverge from each other between 11-24%. FIG. 1B shows the characterization of distinct feature of the synPCCA sequences (SEQ ID NOs: 2-7) and the wild type PCCA gene (SEQ ID NO: 1) using a phylogenetic analysis where distinct grouping is apparent.

TABLE 1
Sequences of wild-type and codon-optimized (or syn) PCCA alleles

| PCCA Allele | Sequences |
|---|---|
| wtPCCA | SEQ ID NO: 1 |
| synPCCA1 | SEQ ID NO: 2 |
| synPCCA2 | SEQ ID NO: 3 |
| synPCCA3 | SEQ ID NO: 4 |
| synPCCA4 | SEQ ID NO: 5 |
| synPCCA5 | SEQ ID NO: 6 |
| synPCCA6 | SEQ ID NO: 7 |

TABLE 2
Sequence alignment of the synthetic PCCA alleles compared to each
other and the wild type PCCA sequence using CLUSTAL multiple
sequence alignment by MUSCLE (3.8)

| | |
|---|---|
| wtPCCA | ATGGCGGGGTTCTGGGTCGGGACAGCACCGCTGGTCGCTGCCGGACGGCGTGGGCGGTGG |
| synPCCA2 | ATGGCCGGGTTTTGGGTGGGCACGGCCCCGCTCGTAGCAGCTGGCAGGCGGGGGCGATGG |
| synPCCA3 | ATGGCCGGCTTCTGGGTGGGGACTGCTCCCCTTGTCGCCGCAGGACGCAGAGGCCGCTGG |
| synPCCA6 | ATGGCCGGATTTTGGGTCGGAACTGCACCACTTGTCGCTGCCGGTAGAAGAGGAAGATGG |
| synPCCA1 | ATGGCAGGGTTCTGGGTCGGCACCGCACCTCTGGTCGCCGCAGGACGCAGGGGAAGATGG |
| synPCCA4 | ATGGCCGGATTTTGGGTTGGAACAGCTCCTCTGGTGGCCGCTGGGAGAAGAGGAAGATGG |
| synPCCA5 | ATGGCCGGCTTCTGGGTGGGCACCGCCCCCCTGGTGGCCGCCGGCAGAAGAGGCAGATGG |
| | ***** ** ** **** ** ** ** ** ** ** ** ** ** * * ** * *** |
| wtPCCA | CCGCCGCAGCAGCTGATGCTGAGCGCGGCGCTGCGGACCCTGAAGCATGTTCTGTACTAT |
| synPCCA2 | CCCCCCCAGCAGCTTATGCTTAGTGCCGCCTTGCGGACGCTGAAGCACGTCCTTTACTAC |
| synPCCA3 | CCTCCTCAGCAGCTCATGCTCTCAGCAGCTCTGAGGACCCTGAAACACGTGCTTTACTAC |
| synPCCA6 | CCACCGCAGCAACTGATGTTGAGCGCTGCACTGCGCACACTGAAGCATGTGCTGTACTAC |
| synPCCA1 | CCTCCACAGCAGCTGATGCTGAGCGCAGCCCTGAGGACCCTGAAGCACGTGCTGTACTAT |
| synPCCA4 | CCTCCTCAGCAGCTGATGCTGTCTGCCGCTCTGAGAACCCTGAAACACGTGCTGTACTAC |
| synPCCA5 | CCCCCCCAGCAGCTGATGCTGAGCGCCGCCCTGAGAACCCTGAAGCACGTGCTGTACTAC |
| | ** ** ***** ****** ** ** ** * ** ***** ** ** ** ***** |
| wtPCCA | TCAAGACAGTGCTTAATGGTGTCCCGTAATCTTGGTTCAGTGGGATATGATCCTAATGAA |
| synPCCA2 | TCTAGACAGTGCCTTATGGTAAGCCGAAATTTGGGAAGTGTAGGTTATGATCCCAACGAG |
| synPCCA3 | AGTCGACAGTGTCTGATGGTGTCTAGGAACCTGGGTAGCGTGGGCTATGATCCCAATGAA |
| synPCCA6 | TCGCGCCAGTGTTTGATGGTGTCCAGGAATCTCGGCTCCGTGGGCTACGACCCCAACGAA |
| synPCCA1 | TCTAGGCAGTGCCTGATGGTCAGCCGCAACCTGGGCAGCGTGGGATACGACCCTAATGAG |
| synPCCA4 | AGCCGGCAGTGCCTGATGGTGTCCAGAAATCTGGGCAGCGTGGGCTACGACCCCAACGAG |
| synPCCA5 | AGCAGACAGTGCCTGATGGTGAGCAGAAACCTGGGCAGCGTGGGCTACGACCCCAACGAG |
| | * ***** * ***** * ** * ** ** ** ** ** ** ** ** |
| wtPCCA | AAAACTTTTGATAAAATTCTTGTTGCTAATAGAGGAGAAATTGCATGTCGGGTTATTAGA |
| synPCCA2 | AAGACCTTTGATAAGATACTGGTTGCTAACCGAGGGGAGATAGCGTGTCGAGTTATTCGC |
| synPCCA3 | AAGACCTTTGACAAAATACTGGTCGCTAATAGAGGGGAAATTGCTTGTCGCGTGATACGG |
| synPCCA6 | AAGACTTTTGACAAGATCCTCGTGGCCAACAGAGGGGAAATTGCGTGCCGCGTGATTCGG |
| synPCCA1 | AAGACATTCGATAAAATCCTGGTGGCTAACCGCGGCGAAATCGCATGCCGAGTGATTCGG |
| synPCCA4 | AAAACCTTCGACAAGATCCTGGTGGCCAACCGGGGAGAGATCGCCTGCAGAGTGATCCGG |
| synPCCA5 | AAGACCTTCGACAAGATCCTGGTGGCCAACAGAGGCGAGATCGCCTGCAGAGTGATCAGA |
| | ** ** ** ** ** ** ** ** ** ** * ** ** ** ** ** * ** ** * |
| wtPCCA | ACTTGCAAGAAGATGGGCATTAAGACAGTTGCCATCCACAGTGATGTTGATGCTAGTTCT |
| synPCCA2 | ACCTGTAAGAAGATGGGAATTAAAACCGTGGCCATCCATAGCGATGTCGACGCTTCCAGT |
| synPCCA3 | ACGTGCAAGAAGATGGGTATCAAAACCGTGGCAATTCACTCTGACGTTGATGCTTCCTCA |
| synPCCA6 | ACTTGCAAGAAGATGGGAATCAAGACCGTGGCCATACACTCCGATGTGGACGCCTCCTCC |
| synPCCA1 | ACCTGTAAGAAAATGGGGATCAAGACAGTCGCCATTCACAGCGACGTGGATGCCAGCAGC |
| synPCCA4 | ACCTGCAAGAAGATGGGCATCAAGACCGTGGCCATCCACTCCGATGTGGATGCCTCTAGC |
| synPCCA5 | ACCTGCAAGAAGATGGGCATCAAGACCGTGGCCATCCACAGCGACGTGGACGCCAGCAGC |
| | ** ** ***** ***** ** ** ** ** ** ** ** ** ** ** ** |
| wtPCCA | GTTCATGTGAAAATGGCGGATGAGGCTGTCTGTGTTGGCCCAGCTCCCACCAGTAAAAGC |
| synPCCA2 | GTGCACGTTAAAATGGCCGACGAGGCCGTATGCGTGGGGCCTGCCCCTACCTCTAAGTCA |
| synPCCA3 | GTGCATGTAAAGATGGCGGATGAGGCTGTTTGCGTGGGTCCAGCACCTACAAGCAAGAGC |
| synPCCA6 | GTCCACGTCAAGATGGCTGATGAAGCCGTCTGCGTGGGACCGGCGCCTACTTCCAAGTCG |
| synPCCA1 | GTCCATGTGAAGATGGCAGACGAGGCCGTCTGCGTGGGACCAGCCCCTACATCTAAAAGT |
| synPCCA4 | GTGCACGTGAAAATGGCCGATGAGGCCGTGTGTGTGGGCCCTGCTCCTACAAGCAAGAGC |
| synPCCA5 | GTGCACGTGAAGATGGCCGACGAGGCCGTGTGCGTGGGCCCCGCCCCCACCAGCAAGAGC |
| | ** ** ** ** ***** ** ** ** ** ** ** ** ** ** ** ** ** |
| wtPCCA | TACCTCAACATGGATGCCATCATGGAAGCCATTAAGAAAACCAGGGCCCAAGCTGTACAT |
| synPCCA2 | TACCTGAACATGGATGCAATTATGGAAGCTATTAAGAAGACTCGGGCGCAGGCTGTCCAC |
| synPCCA3 | TATCTCAACATGGATGCCATCATGGAAGCTATCAAGAAAACCCGTGCACAAGCTGTGCAT |
| synPCCA6 | TACCTTAACATGGACGCCATCATGGAGGCCATCAAGAAAACCAGGGCGCAGGCGGTGCAT |
| synPCCA1 | TACCTGAACATGGATGCTATCATGGAAGCAATTAAGAAAACTAGGGCCCAGGCTGTGCAC |
| synPCCA4 | TACCTGAACATGGACGCCATCATGGAAGCCATTAAGAAAACAAGAGCCCAGGCCGTGCAT |
| synPCCA5 | TACCTGAACATGGACGCCATCATGGAGGCCATCAAGAAGACCAGAGCCCAGGCCGTGCAC |
| | ** ** ******** ** ** ***** ** ** ***** ** * ** ** ** ** ** |
| wtPCCA | CCAGGTTATGGATTCCTTTCAGAAAACAAAGAATTTGCCAGATGTTTGGCAGCAGAAGAT |
| synPCCA2 | CCTGGATATGGATTTCTTTCTGAGAATAAGGAGTTTGCCCGGTGTCTGGOGGCAGAAGAC |
| synPCCA3 | CCAGGGTATGGCTTTCTCTCCGAGAACAAAGAATTTGCCCGGTGTCTGGCAGCGGAGGAC |
| synPCCA6 | CCTGGCTACGGCTTCCTGTCCGAAAACAAGGAGTTCGCACGGTGCCTGGCCGCCGAGGAC |
| synPCCA1 | CCTGGCTATGGGTTCCTGAGCGAGAATAAGGAATTTGCACGATGTCTGGCAGCTGAGGAC |
| synPCCA4 | CCCGGCTACGGATTTCTGAGCGAGAACAAAGAATTTGCCCGGTGCCTGGCCGCCGAGGAC |
| synPCCA5 | CCCGGCTACGGCTTCCTGAGCGAGAACAAGGAGTTCGCCAGATGCCTGGCCGCCGAGGAC |
| | ** ** ** ** ** ** ** ** ** ** ** ** * ** **** ** ** ** |
| wtPCCA | GTCGTTTTCATTGGACCTGACACACATGCTATTCAAGCCATGGGCGACAAGATTGAAAGC |
| synPCCA2 | GTCGTATTCATTGGACCGGATACGCACGCTATCCAAGCCATGGGAGATAAGATCGAGAGC |
| synPCCA3 | GTGGTGTTCATTGGGCCTGATACGCATGCAATTCAAGCCATGGGCGATAAGATTGAGAGC |
| synPCCA6 | GTGGTCTTTATCGGGCCCGACACCCATGCAATCCAGGCCATGGGCGACAAGATCGAGTCG |
| synPCCA1 | GTGGTCTTTATCGGACCAGATACACATGCTATTCAGGCAATGGGCGACAAGATCGAGTCC |
| synPCCA4 | GTGGTGTTTATTGGCCCTGATACACACGCCATCCAGGCCATGGGCGATAAGATCGAGTCT |
| synPCCA5 | GTGGTGTTCATCGGCCCCGACACCCACGCCATCCAGGCCATGGGCGACAAGATCGAGAGC |
| | ** ** ** ** ** ** ** ** ** ** ** ** ** ***** ** ***** ** |
| wtPCCA | AAATTATTAGCTAAGAAAGCAGAGGTTAATACAATCCCTGGCTTTGATGGAGTAGTCAAG |
| synPCCA2 | AAGCTCCTGGCTAAGAAAGCTGAAGTGAACACCATTCCTGGCTTTGACGGCGTGGTGAAG |
| synPCCA3 | AAGCTGCTTGCTAAGAAAGCAGAAGTTAACACAATCCCAGGCTTTGACGGCGTTGTCAAA |
| synPCCA6 | AAGCTGCTGGCGAAGAAGGCAGAAGTGAACACTATTCCCGGGTTCGACGGAGTGGTCAAA |
| synPCCA1 | AAACTGCTGGCCAAGAAAGCTGAAGTGAATACTATCCCCGGGTTCGACGGAGTGGTCAAG |
| synPCCA4 | AAGCTGCTGGCCAAGAAAGCCGAAGTGAACACAATCCCCGGCTTCGACGGCGTGGTCAAG |
| synPCCA5 | AAGCTGCTGGCCAAGAAGGCCGAGGTGAACACCATCCCCGGCTTCGACGGCGTGGTGAAG |
| | ** * * ** ***** ** ** ** ** ** ** ** ** ** ** ** ** ** ** |
| wtPCCA | GATGCAGAAGAAGCTGTCAGAATTGCAAGGGAAATTGGCTACCCTGTCATGATCAAGGCC |
| synPCCA2 | GACGCAGAGGAAGCTGTTCGCATCGCCCGCGAAATTGGATATCCCGTGATGATAAAAGCA |
| synPCCA3 | GACGCCGAAGAAGCGGTACGTATTGCCCGAGAAATCGGCTACCCCGTTATGATCAAGGCG |
| synPCCA6 | GACGCGGAAGAGGCCGTCCGAATCGCCCGGGAGATTGGATACCCTGTGATGATTAAGGCC |
| synPCCA1 | GATGCAGAGGAAGCCGTGAGAATCGCCAGGGAGATTGGCTACCCTGTGATGATTAAGGCA |
| synPCCA4 | GATGCTGAAGAAGCCGTGCGGATCGCCAGAGAAATCGGCTACCCCGTGATGATCAAAGCC |
| synPCCA5 | GACGCCGAGGAGGCCGTGAGAATCGCCAGAGAGATCGGCTACCCCGTGATGATCAAGGCC |
| | ** ** ** ** ** ** * ** ** * ** ** ** ** ** ** ***** ** ** |
| wtPCCA | TCAGCAGGTGGTGGTGGGAAAGGCATGCGCATTGCTTGGGATGATGAAGAGACCAGGGAT |
| synPCCA2 | TCTGCGGGGGGGGGGGGGAAGGGCATGAGAATTGCCTGGGATGATGAAGAAACTAGAGAT |
| synPCCA3 | TCAGCCGGAGGTGGAGGAAAAGGGATGAGGATTGCCTGGGATGACGAGGAGACTAGGGAT |
| synPCCA6 | TCGGCTGGCGGAGGCGGAAAGGGAATGCGCATTGCCTGGGATGACGAAGAAACCCGGGAT |
| synPCCA1 | TCTGCCGGCGGGGGAGGCAAAGGGATGAGGATCGCCTGGGACGATGAGGAAACTCGCGAT |
| synPCCA4 | TCTGCTGGCGGAGGCGGCAAGGGAATGAGAATCGCCTGGGACGACGAAGAGACACGCGAC |
| synPCCA5 | AGCGCCGGCGGCGGCGGCAAGGGCATGAGAATCGCCTGGGACGACGAGGAGACCAGAGAC |
| | ** ** ** ** ** ** ** *** * ** ** ***** ** ** ** ** * ** |
| wtPCCA | GGTTTTAGATTGTCATCTCAAGAAGCTGCTTCTAGTTTTGGCGATGATAGACTACTAATA |
| synPCCA2 | GGTTTCCGCTTGTCTTCTCAGGAAGCCGCATCATCCTTTGGAGATGACCGATTGCTCATA |
| synPCCA3 | GGGTTCCGGCTCTCCAGTCAGGAAGCAGCATCTTCTTTTGGTGACGATAGACTGCTGATA |
| synPCCA6 | GGATTCCGGCTGAGCTCCCAAGAAGCCGCATCGTCCTTCGGGGACGATAGACTGCTGATC |
| synPCCA1 | GGATTTCGACTGTCTAGTCAGGAAGCAGCCAGCAGCTTCGGCGACGATAGGCTGCTGATC |
| synPCCA4 | GGCTTTAGACTGAGCAGCCAAGAAGCCGCCAGCTCCTTCGGAGATGACAGACTGCTGATC |
| synPCCA5 | GGCTTCAGACTGAGCAGCCAGGAGGCCGCCAGCAGCTTCGGCGACGACAGACTGCTGATC |
| | ** ** * * ** ** ** ** ** ** ** ** * * ** ** |
| wtPCCA | GAAAAATTTATTGATAATCCTCGTCATATAGAAATCCAGGTTCTAGGTGATAAACATGGG |
| synPCCA2 | GAGAAATTTATCGACAATCCACGGCATATTGAGATCCAAGTGCTTGGCGACAAGCACGGT |
| synPCCA3 | GAGAAATTCATCGACAACCCTCGACACATTGAAATCCAGGTACTGGGAGACAAACACGGA |
| synPCCA6 | GAAAAGTTCATCGACAACCCAAGGCACATCGAAATCCAGGTCCTCGGGGACAAGCATGGA |
| synPCCA1 | GAGAAGTTCATTGACAACCCCCGCCACATCGAAATTCAGGTGCTGGGGGATAAACATGGA |
| synPCCA4 | GAGAAGTTCATCGACAACCCCAGACACATCGAGATCCAGGTGCTGGGCGACAAGCACGGA |
| synPCCA5 | GAGAAGTTCATCGACAACCCCAGACACATCGAGATCCAGGTGCTGGGCGACAAGCACGGC |
| | ** ** ** ** ** ** ** * ** ** ** ** ** ** ** ** ** ** ** ** |
| wtPCCA | AATGCTTTATGGCTTAATGAAAGAGAGTGCTCAATTCAGAGAAGAAATCAGAAGGTGGTG |
| synPCCA2 | AACGCGCTTTGGCTCAACGAACGAGAGTGTTCAATCCAGAGGAGGAACCAGAAGGTTGTA |
| synPCCA3 | AATGCACTTTGGCTCAATGAACGCGAGTGCTCCATTCAGCGCAGGAACCAGAAAGTGGTC |
| synPCCA6 | AACGCCCTGTGGTTGAACGAGAGAGAGTGCTCCATTCAACGGCGCAACCAGAAGGTCGTG |
| synPCCA1 | AACGCCCTGTGGCTGAATGAGCGGGAATGTAGCATTCAGCGGAGAAATCAGAAGGTGGTC |
| synPCCA4 | AATGCCCTGTGGCTGAACGAGAGAGAGTGCAGCATCCAGCGGCGGAACCAGAAAGTGGTG |
| synPCCA5 | AACGCCCTGTGGCTGAACGAGAGAGAGTGCAGCATCCAGAGAAGAAACCAGAAGGTGGTG |
| | ** ** * *** * ** ** * ** ** ** ** * * ** ***** ** ** |
| wtPCCA | GAGGAAGCACCAAGCATTTTTTTGGATGCGGAGACTCGAAGAGCGATGGGAGAACAAGCT |
| synPCCA2 | GAAGAAGCACCATCTATTTTCCTCGACGCAGAAACTCGGCGGGCTATGGGGGAACAAGCA |
| synPCCA3 | GAGGAAGCACCCTCCATCTTCCTGGATGCCGAGACAAGGCGCGCTATGGGCGAGCAGGCC |
| synPCCA6 | GAGGAAGCCCCCTCGATTTTCCTCGATGCTGAAACTCGCCGGGCCATGGGGGAGCAAGCG |
| synPCCA1 | GAGGAAGCTCCTTCCATCTTTCTGGACGCCGAGACAAGGCGCGCTATGGGAGAACAGGCT |
| synPCCA4 | GAAGAGGCCCCTAGCATCTTCCTGGACGCCGAAACTCGGAGAGCCATGGGAGAACAGGCT |
| synPCCA5 | GAGGAGGCCCCCAGCATCTTCCTGGACGCCGAGACCAGAAGAGCCATGGGCGAGCAGGCC |
| | ** ** ** ** ** ** * ** ** ** ** * * ** ***** ** ** ** |
| wtPCCA | GTAGCTCTTGCCAGAGCAGTAAAATATTCCTCTGCTGGGACCGTGGAGTTCCTTGTGGAC |
| synPCCA2 | GTGGCACTGGCTCGAGCCGTTAAATATTCTAGTGCGGGGACAGTAGAATTCCTCGTAGAT |
| synPCCA3 | GTTGCACTCGCTAGAGCCGTGAAGTACTCTTCTGCGGGTACCGTGGAATTTCTGGTAGAC |
| synPCCA6 | GTGGCCCTGGCCCGCGCAGTGAAGTACTCCTCGGCCGGGACCGTGGAGTTCCTGGTGGAC |
| synPCCA1 | GTCGCACTGGCCAGAGCTGTGAAATACTCCTCTGCCGGCACTGTCGAGTTCCTGGTGGAC |
| synPCCA4 | GTGGCTCTGGCTAGAGCCGTGAAGTATAGCAGCGCCGGCACCGTGGAATTTCTGGTGGAC |
| synPCCA5 | GTGGCCCTGGCCAGAGCCGTGAAGTACAGCAGCGCCGGCACCGTGGAGTTCCTGGTGGAC |
| | ** ** ** ** * ** ** ** ** ** ** ** ** ** ** ** ** ** |
| wtPCCA | TCTAAGAAGAATTTTTATTTCTTGGAAATGAATACAAGACTCCAGGTTGAGCATCCTGTC |
| synPCCA2 | AGCAAGAAGAATTTTTATTTTCTTGAGATGAATACGCGCCTTCAAGTGGAACACCCAGTC |
| synPCCA3 | AGCAAGAAGAACTTCTATTTCCTGGAGATGAATACCCGGCTGCAAGTCGAGCATCCAGTC |
| synPCCA6 | AGCAAAAAGAACTTCTACTTTCTCGAGATGAACACCAGGCTCCAAGTGGAGCACCCTGTG |
| synPCCA1 | AGCAAGAAAAACTTCTATTTTCTGGAAATGAACACCCGGCTGCAGGTCGAGCACCCAGTG |
| synPCCA4 | AGCAAGAAGAACTTCTACTTCCTCGAGATGAACACCCGGCTGCAGGTCGAGCACCCTGTG |
| synPCCA5 | AGCAAGAAGAACTTCTACTTCCTGGAGATGAACACCAGACTGCAGGTGGAGCACCCCGTG |
| | ** ** ** ** ** ** * ** ***** ** * ** ** ** ** ** ** ** |
| wtPCCA | ACAGAATGCATTACTGGCCTGGACCTAGTCCAGGAAATGATCCGTGTTGCTAAGGGCTAC |
| synPCCA2 | ACGGAATGTATAACTGGCCTTGACTTGGTTCAGGAGATGATACGGGTGGCTAAGGGTTAT |
| synPCCA3 | ACTGAGTGTATAACTGGCCTGGACCTGGTACAGGAAATGATTCGTGTAGCGAAGGGATAC |
| synPCCA6 | ACCGAATGCATCACTGGACTTGACCTGGTGCAGGAAATGATCCGCGTGGCCAAGGGATAC |
| synPCCA1 | ACTGAATGCATTACCGGGCTGGATCTGGTCCAGGAGATGATCAGAGTGGCCAAGGGATAC |
| synPCCA4 | ACCGAGTGTATCACAGGCCTGGACCTGGTGCAAGAGATGATCAGAGTGGCCAAGGGCTAC |
| synPCCA5 | ACCGAGTGCATCACCGGCCTGGACCTGGTGCAGGAGATGATCAGAGTGGCCAAGGGCTAC |
| | ** ** ** ** ** ** ** ** * ** ** ** ***** * ** ** ***** ** |
| wtPCCA | CCTCTCAGGCACAAACAAGCTGATATTCGCATCAACGGCTGGGCAGTTGAATGTCGGGTT |
| synPCCA2 | CCTCTTCGGCATAAGCAGGCTGATATTCGCATAAATGGGTGGGGGGTCGAGTGCAGAGTT |
| synPCCA3 | CCGCTCCGGCACAAACAAGCCGACATTCGCATCAATGGGTGGGCTGTGGAGTGCAGAGTC |
| synPCCA6 | CCCCTGAGGCACAAGCAGGCCGACATCAGAATCAACGGTTGGGCCGTGGAATGTCGGGTG |
| synPCCA1 | CCCCTGCGACATAAACAGGCTGACATCCGGATTAACGGCTGGGCAGTCGAGTGTCGGGTG |
| synPCCA4 | CCTCTGAGACACAAGCAGGCCGACATCCGGATCAATGGCTGGGCCGTTGAGTGCAGAGTG |
| synPCCA5 | CCCCTGAGACACAAGCAGGCCGACATCAGAATCAACGGCTGGGCCGTGGAGTGCAGAGTG |
| | ** ** * ** ** ** ** ** ** * ** ** ** ***** ** ** ** * ** |
| wtPCCA | TATGCTGAGGACCCCTACAAGTCTTTTGGTTTACCATCTATTGGGAGATTGTCTCAGTAC |
| synPCCA2 | TATGCTGAGGACCCATACAAGTCATTCGGACTTCCTTCTATAGGCAGACTGTCACAATAT |
| synPCCA3 | TATGCAGAGGATCCCTATAAGTCCTTCGGGCTTCCCTCCATAGGCAGGCTTAGTCAGTAT |
| synPCCA6 | TACGCTGAGGATCCGTATAAGTCCTTCGGCTTGCCGAGCATCGGACGGCTGTCACAGTAC |
| synPCCA1 | TACGCCGAAGATCCATATAAGTCTTTCGGACTGCCCAGTATTGGCCGACTGTCACAGTAT |
| synPCCA4 | TACGCCGAGGATCCCTACAAGAGCTTCGGCCTGCCTAGCATCGGCCGGCTGTCTCAGTAT |
| synPCCA5 | TACGCCGAGGACCCCTACAAGAGCTTCGGCCTGCCCAGCATCGGCAGACTGAGCCAGTAC |
| | ** ** ** ** ** ** *** ** ** * ** ** ** * * ** ** |
| wt PCCA | CAAGAACCGTTACATCTACCTGGTGTCCGAGTGGACAGTGGCATCCAACCAGGAAGTGAT |
| synPCCA2 | CAAGAGCCACTTCATCTCCCAGGTGTAAGAGTAGATTCCGGAATACAACCTGGCTCCGAT |
| synPCCA3 | CAGGAGCCATTGCACTTGCCTGGCGTCAGGGTGGACTCCGGCATCCAACCGGGCAGCGAC |
| synPCCA6 | CAGGAACCCCTGCACCTTCCTGGAGTCAGAGTGGACTCCGGAATCCAACCTGGTTCGGAC |
| synPCCA1 | CAGGAGCCTCTGCACCTGCCAGGCGTCAGAGTGGACAGCGGCATCCAGCCTGGGTCCGAC |
| synPCCA4 | CAAGAGCCACTGCATCTGCCCGGCGTCAGAGTGGATTCTGGAATCCAGCCTGGCAGCGAC |
| synPCCA5 | CAGGAGCCCCTGCACCTGCCCGGCGTGAGAGTGGACAGCGGCATCCAGCCCGGCAGCGAC |
| | ** ** ** * ** * ** ** ** * ** ** ** ** ** ** ** ** |
| wtPCCA | ATTAGCATTTATTATGATCCTATGATTTCAAAACTAATCACATATGGCTCTGATAGAACT |
| synPCCA2 | ATATCTATTTACTATGATCCAATGATTAGTAAGTTGATTACATATGGGAGTGATCGGACC |
| synPCCA3 | ATTTCAATTTACTACGATCCCATGATCAGCAAGTTGATTACCTATGGATCTGACCGGACA |
| synPCCA6 | ATTTCCATCTACTACGATCCGATGATCTCCAAACTCATTACCTACGGTAGCGACCGGACC |
| synPCCA1 | ATCTCTATCTACTATGATCCAATGATCAGCAAGCTGATTACATACGGCTCCGATCGGACT |
| synPCCA4 | ATCAGCATCTACTACGACCCTATGATCTCCAAGCTGATCACCTACGGCAGCGACCGGACA |
| synPCCA5 | ATCAGCATCTACTACGACCCCATGATCAGCAAGCTGATCACCTACGGCAGCGACAGAACC |
| | ** ** ** ** ** ** ***** ** * ** ** ** ** ** * ** |
| wtPCCA | GAGGCACTGAAGAGAATGGCAGATGCACTGGATAACTATGTTATTCGAGGTGTTACACAT |
| synPCCA2 | GAAGCTTTGAAGCGGATGGCGGACGCGCTGGATAACTACGTGATAAGGGGTGTCACGCAC |
| synPCCA3 | GAGGCTCTGAAGAGAATGGCCGACGCCCTGGACAATTACGTGATAAGAGGAGTGACACAC |
| synPCCA6 | GAGGCTCTGAAACGCATGGCTGACGCCCTGGACAACTATGTCATCCGGGGAGTCACTCAC |
| synPCCA1 | GAGGCCCTGAAAAGAATGGCAGACGCCCTGGATAACTATGTCATTAGAGGGGTGACCCAT |
| synPCCA4 | GAGGCCCTGAAGAGAATGGCTGACGCCCTGGACAACTACGTGATCAGAGGCGTGACCCAC |
| synPCCA5 | GAGGCCCTGAAGAGAATGGCCGACGCCCTGGACAACTACGTGATCAGAGGCGTGACCCAC |
| | ** ** **** * ***** ** ** ***** ** ** ** ** * ** ** ** ** |
| wtPCCA | AATATTGCATTACTTCGAGAGGTGATAATCAACTCACGCTTTGTAAAAGGAGACATCAGC |
| synPCCA2 | AATATAGCTCTGCTGAGGGAGGTAATTATCAACAGTCGGTTCGTGAAGGGTGACATTAGC |
| synPCCA3 | AACATTGCCCTGTTGCGGGAGGTGATCATCAATAGCAGATTCGTGAAGGGTGACATCTCC |
| synPCCA6 | AATATCGCGCTGCTGCGCGAAGTCATCATTAATAGCCGCTTCGTGAAGGGCGACATTTCC |
| synPCCA1 | AATATCGCTCTGCTGAGAGAAGTCATCATTAACTCCAGGTTCGTGAAGGGAGACATCAGC |
| synPCCA4 | AATATCGCCCTGCTGCGGGAAGTGATCATCAACAGCAGATTCGTGAAAGGCGATATCAGC |
| synPCCA5 | AACATCGCCCTGCTGAGAGAGGTGATCATCAACAGCAGATTCGTGAAGGGCGACATCAGC |
| | ** ** ** * * * ** ** ** ** ** * ** ** ** ** ** ** * |
| wtPCCA | ACTAAATTTCTCTCCGATGTGTATCCTGATGGCTTCAAAGGACACATGCTAACCAAGAGT |
| synPCCA2 | ACTAAGTTCCTCTCCGACGTGTACCCAGACGGTTTTAAAGGGCACATGCTTACTAAGTCC |
| synPCCA3 | ACCAAGTTCCTGAGTGACGTATACCCCGACGGCTTTAAGGGGCATATGCTGACAAAGTCA |
| synPCCA6 | ACCAAGTTCCTGAGCGACGTGTACCCTGATGGTTTCAAGGGTCACATGCTGACTAAGTCC |
| synPCCA1 | ACCAAATTTCTGTCCGACGTGTACCCCGATGGCTTCAAGGGGCACATGCTGACAAAGTCT |
| synPCCA4 | ACCAAGTTTCTGTCCGACGTGTACCCCGACGGCTTCAAGGGACACATGCTGACCAAGAGC |
| synPCCA5 | ACCAAGTTCCTGAGCGACGTGTACCCCGACGGCTTCAAGGGCCACATGCTGACCAAGAGC |
| | ** ** ** ** ** ** ** ** ** ** ** ** ** ** ***** ** *** |
| wtPCCA | GAGAAGAACCAGTTATTGGCAATAGCATCATCATTGTTTGTGGCATTCCAGTTAAGAGCA |
| synPCCA2 | GAAAAGAATCAACTGTTGGCTATTGCGTCTTCCCTTTTTGTTGCTTTCCAACTGCGCGCG |
| synPCCA3 | GAGAAGAATCAACTCCTCGCAATAGCCAGTAGCCTGTTTGTTGCCTTCCAGCTGAGGGCT |
| synPCCA6 | GAGAAGAACCAGCTCCTCGCTATCGCGTCCTCCCTGTTTGTGGCGTTCCAGCTGAGGGCC |
| synPCCA1 | GAGAAAAATCAGCTGCTGGCTATCGCAAGTTCACTGTTCGTGGCATTTCAGCTGCGGGCC |
| synPCCA4 | GAGAAGAACCAGCTGCTCGCCATTGCCTCCAGCCTGTTTGTGGCCTTTCAGCTGAGAGCC |
| synPCCA5 | GAGAAGAACCAGCTGCTGGCCATCGCCAGCAGCCTGTTCGTGGCCTTCCAGCTGAGAGCC |
| | ** ** ** ** * * ** ** ** * ** ** ** ** ** * * ** |
| wtPCCA | CAACATTTTCAAGAAAATTCAAGAATGCCTGTTATTAAACCAGACATAGCCAACTGGGAG |
| synPCCA2 | CAGCATTTCCAGGAGAATAGCAGAATGCCCGTTATCAAACCTGATATTGCGAACTGGGAA |
| synPCCA3 | CAGCACTTCCAGGAGAATAGCAGAATGCCCGTTATCAAACCTGATATCGCGAATTGGGAA |
| synPCCA6 | CAGCACTTCCAAGAAAACTCAAGAATGCCGGTCATCAAGCCCGACATTGCCAATTGGGAA |
| synPCCA1 | CAGCATTTTCAGGAGAACAGTAGAATGCCCGTGATCAAGCCTGACATTGCAAATTGGGAA |
| synPCCA4 | CAGCACTTCCAAGAGAACAGCAGAATGCCCGTGATCAAGCCCGATATCGCCAACTGGGAG |
| synPCCA5 | CAGCACTTCCAGGAGAACAGCAGAATGCCCGTGATCAAGCCCGACATCGCCAACTGGGAG |
| | ** ** ** ** ** ** ******** ** ** ** ** ** ** ** ** ***** |
| wtPCCA | CTCTCAGTAAAATTGCATGATAAAGTTCATACCGTAGTAGCATCAAACAATGGGTCAGTG |
| synPCCA2 | TTGTCAGTTAAGCTGCATGATAAGGTGCATACCGTAGTGGCTAGTAATAACGGAAGCGTT |
| synPCCA3 | TTGAGCGTGAAGCTGCACGATAAAGTTCATACTGTTGTGGCCTCAAACAATGGAAGCGTC |
| synPCCA6 | CTGAGCGTGAAGCTGCACGACAAAGTGCACACCGTGGTGGCCAGCAACAACGGCTCCGTG |
| synPCCA1 | CTGAGTGTCAAGCTGCACGATAAAGTGCATACCGTGGTCGCTTCAAACAATGGCAGCGTG |
| synPCCA4 | CTGAGCGTGAAGCTGCACGATAAGGTGCACACAGTGGTGGCCAGCAACAACGGCTCCGTG |
| synPCCA5 | CTGAGCGTGAAGCTGCACGACAAGGTGCACACCGTGGTGGCCAGCAACAACGGCAGCGTG |
| | * ** ** **** ** ** ** ** ** ** ** ** ** ** ** ** |
| wtPCCA | TTCTCGGTGGAAGTTGATGGGTCGAAACTAAATGTGACCAGCACGTGGAACCTGGCTTCG |
| synPCCA2 | TTTTCCGTTGAAGTAGACGGCTCCAAGCTTAATGTGACGAGCACATGGAACCTTGCCTCT |
| synPCCA3 | TTTAGCGTGGAGGTCGATGGATCCAAACTGAACGTGACCAGTACCTGGAATTTGGCCAGT |
| synPCCA6 | TTCTCCGTGGAAGTGGATGGGTCAAAGCTGAACGTGACCAGCACCTGGAACCTGGCGTCC |
| synPCCA1 | TTCAGCGTCGAGGTGGACGGGTCTAAACTGAACGTGACCAGTACATGGAATCTGGCCTCA |
| synPCCA4 | TTCAGCGTGGAAGTGGACGGCAGCAAGCTGAACGTGACCTCCACCTGGAATCTGGCCTCT |
| synPCCA5 | TTCAGCGTGGAGGTGGACGGCAGCAAGCTGAACGTGACCAGCACCTGGAACCTGGCCAGC |
| | ** ** ** ** ** ** ** ** ** ***** ** ***** * ** |
| wtPCCA | CCCTTATTGTCTGTCAGCGTTGATGGCACTCAGAGGACTGTCCAGTGTCTTTCTCGAGAA |
| synPCCA2 | CCACTGCTTAGTGTGAGTGTGGACGGAACGCAGAGGACAGTTCAATGCCTGAGTCGGGAA |
| synPCCA3 | CCGCTGTTGTCTGTCTCCGTGGATGGAACGCAACGAACTGTGCAGTGTCTGTCTCGCGAA |
| synPCCA6 | CCGCTCCTGTCAGTGTCCGTGGACGGCACTCAGCGGACTGTGCAGTGTTTGTCCCGGGAA |
| synPCCA1 | CCACTGCTGTCAGTCAGCGTGGATGGCACACAGCGCACTGTGCAGTGCCTGAGCCGGGAG |
| synPCCA4 | CCACTGCTGTCCGTGTCTGTGGATGGCACCCAGAGAACCGTGCAGTGTCTGAGCAGAGAA |
| synPCCA5 | CCCCTGCTGAGCGTGAGCGTGGACGGCACCCAGAGAACCGTGCAGTGCCTGAGCAGAGAG |
| | ** * * ** ** ** ** ** ** * ** ** ** ** * * ** |
| wtPCCA | GCAGGTGGAAACATGAGCATTCAGTTTCTTGGTACAGTGTACAAGGTGAATATCTTAACC |
| synPCCA2 | GCGGGAGGTAACATGAGTATACAATTCCTCGGAACCGTCTATAAAGTTAACATTTTGACG |
| synPCCA3 | GCCGGAGGCAACATGAGCATTCAGTTTCTCGGGACTGTGTACAAAGTCAACATCCTGACC |
| synPCCA6 | GCCGGGGGCAATATGAGCATCCAGTTCCTCGGGACGGTGTACAAGGTCAACATCCTCACT |
| synPCCA1 | GCAGGAGGAAACATGAGTATTCAGTTTCTGGGGACTGTCTATAAGGTGAACATCCTGACC |
| synPCCA4 | GCAGGCGGCAATATGAGCATCCAGTTTCTGGGCACCGTGTACAAAGTGAACATCCTGACC |
| synPCCA5 | GCCGGCGGCAACATGAGCATCCAGTTCCTGGGCACCGTGTACAAGGTGAACATCCTGACC |
| | ** ** ** ** ***** ** ** ** ** ** ** ** ** ** ** ** ** * ** |
| wtPCCA | AGACTTGCCGCAGAATTGAACAAATTTATGCTGGAAAAAGTGACTGAGGACACAAGCAGT |
| synPCCA2 | AGATTGGCGGCTGAACTGAATAAGTTCATGCTCGAGAAAGTGACTGAGGACACTTCAAGC |
| synPCCA3 | CGACTGGCTGCCGAGCTGAACAAATTTATGCTTGAGAAAGTCACTGAGGATACGTCTAGC |
| synPCCA6 | CGGTTGGCCGCTGAACTCAACAAGTTCATGCTGGAAAAGGTCACCGAGGACACCTCCTCT |
| synPCCA1 | AGGCTGGCTGCAGAACTGAATAAGTTCATGCTGGAGAAAGTGACCGAAGACACAAGCTCC |
| synPCCA4 | AGACTGGCCGCTGAGCTGAACAAGTTCATGCTGGAAAAAGTGACCGAGGACACCAGCAGC |
| synPCCA5 | AGACTGGCCGCCGAGCTGAACAAGTTCATGCTGGAGAAGGTGACCGAGGACACCAGCAGC |
| | * * ** ** ** * ** ** ** ***** ** ** ** ** ** ** ** |
| wtPCCA | GTTCTGCGTTCCCCGATGCCCGGAGTGGTGGTGGCCGTCTCTGTCAAGCCTGGAGACGCG |
| synPCCA2 | GTACTGAGGAGCCCTATGCCGGGGGTTGTCGTAGCAGTGTCTGTTAAGCCAGGAGATGCG |
| synPCCA3 | GTCCTTCGGAGTCCTATGCCAGGGGTGGTGGTGGCCGTTTCAGTCAAACCAGGTGATGCC |
| synPCCA6 | GTGCTGCGGTCGCCCATGCCGGGAGTGGTCGTGGCCGTGTCCGTGAAGCCTGGCGATGCC |
| synPCCA1 | GTGCTGCGCTCACCAATGCCAGGAGTGGTCGTGGCCGTCAGCGTGAAGCCAGGGGATGCA |
| synPCCA4 | GTGCTGAGATCTCCTATGCCTGGTGTCGTGGTGGCCGTGTCAGTGAAACCTGGGGATGCT |
| synPCCA5 | GTGCTGAGAAGCCCCATGCCCGGCGTGGTGGTGGCCGTGAGCGTGAAGCCCGGCGACGCC |
| | ** ** * ** ***** ** ** ** ** ** ** ** ** ** ** ** ** |
| wtPCCA | GTAGCAGAAGGTCAAGAAATTTGTGTGATTGAAGCCATGAAAATGCAGAATAGTATGACA |
| synPCCA2 | GTGGCAGAAGGCCAAGAAATTTGCGTGATTGAGGCAATGAAAATGCAGAACTCAATGACC |
| synPCCA3 | GTAGCCGAAGGTCAGGAAATCTGCGTTATCGAGGCTATGAAGATGCAGAACAGCATGACA |
| synPCCA6 | GTGGCCGAAGGTCAAGAAATTTGCGTGATCGAGGCCATGAAGATGCAGAACTCGATGACG |
| synPCCA1 | GTGGCTGAGGGACAGGAGATTTGCGTGATTGAGGCTATGAAAATGCAGAACAGCATGACC |
| synPCCA4 | GTGGCCGAGGGCCAAGAGATCTGTGTGATCGAGGCCATGAAGATGCAGAACAGCATGACC |
| synPCCA5 | GTGGCCGAGGGCCAGGAGATCTGCGTGATCGAGGCCATGAAGATGCAGAACAGCATGACC |
| | ** ** ** ** ** ** ** ** ** ** ** ** ***** ******** ***** |
| wtPCCA | GCTGGGAAAACTGGCACGGTGAAATCTGTGCACTGTCAAGCTGGAGACACAGTTGGAGAA |
| synPCCA2 | GCCGGAAAAACGGGCACGGTCAAATCTGTGCATTGTCAGGCAGGCGACACAGTCGGCGAG |
| synPCCA3 | GCCGGGAAAACCGGAACAGTGAAGTCAGTTCATTGCCAGGCTGGGGACACAGTCGGCGAG |
| synPCCA6 | GCCGGAAAGACCGGCACCGTCAAAAGCGTGCACTGCCAGGCCGGCGATACCGTGGGAGAG |
| synPCCA1 | GCAGGAAAGACTGGCACCGTGAAAAGCGTGCATTGTCAGGCTGGGGATACTGTCGGGGAA |
| synPCCA4 | GCCGGCAAGACCGGCACAGTGAAGTCTGTGCATTGTCAGGCCGGCGATACAGTCGGAGAA |
| synPCCA5 | GCCGGCAAGACCGGCACCGTGAAGAGCGTGCACTGCCAGGCCGGCGACACCGTGGGCGAG |
| | ** ** ** ** ** ** ** ** ** ** ** ** ** ** ** ** ** ** ** |
| wtPCCA | GGGGATCTGCTCGTGGAGCTGGAATGA |
| synPCCA2 | GGTGATCTCCTGGTAGAGTTGGAATGA |
| synPCCA3 | GGCGATTTGCTGGTGGAACTGGAATGA |
| synPCCA6 | GGCGATCTGCTCGTGGAACTCGAATGA |
| synPCCA1 | GGGGATCTGCTGGTGGAACTGGAGTGA |
| synPCCA4 | GGCGATCTGCTGGTGGAACTGGAATGA |
| synPCCA5 | GGCGACCTGCTGGTGGAGCTGGAGTGA |
| | ** ** * ** ** ** * ** *** |

In another aspect, SEQ ID NOs:2-7 encode a PCC alpha subunit that has 100% identity with the naturally occurring human PCC alpha subunit protein, or that has at least 90% amino acid identity to the naturally occurring human PCC alpha subunit protein. In a preferred aspect, the polynucleotide encodes a PCC alpha subunit protein that has at least 95% amino acid identity to naturally occurring human PCC alpha subunit protein.

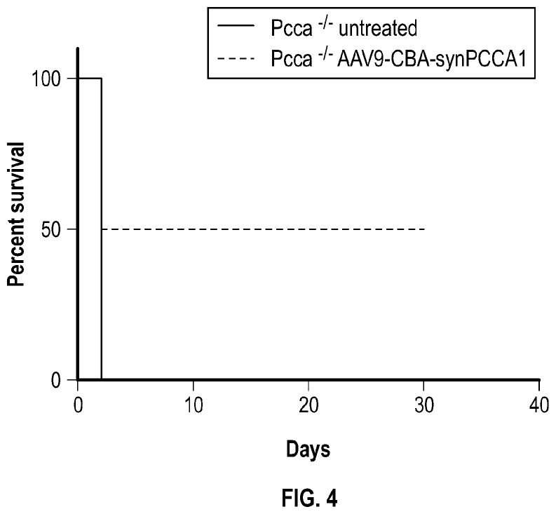

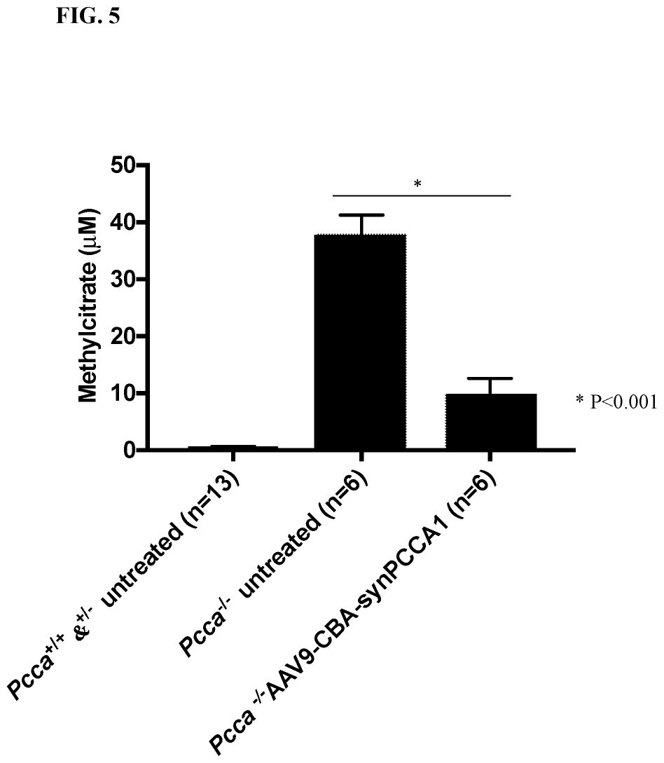

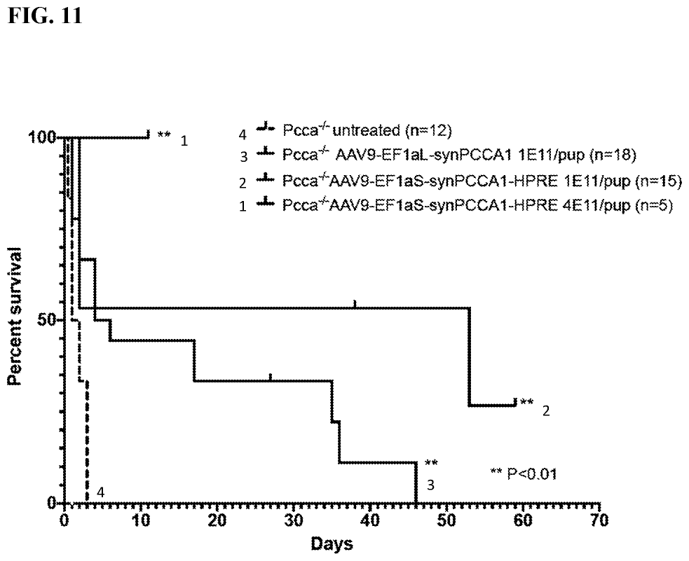

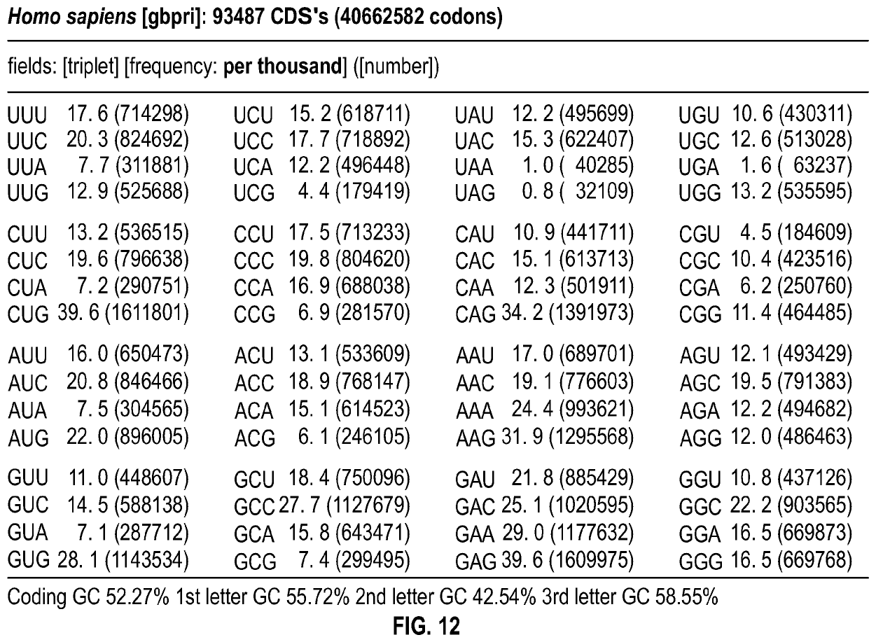

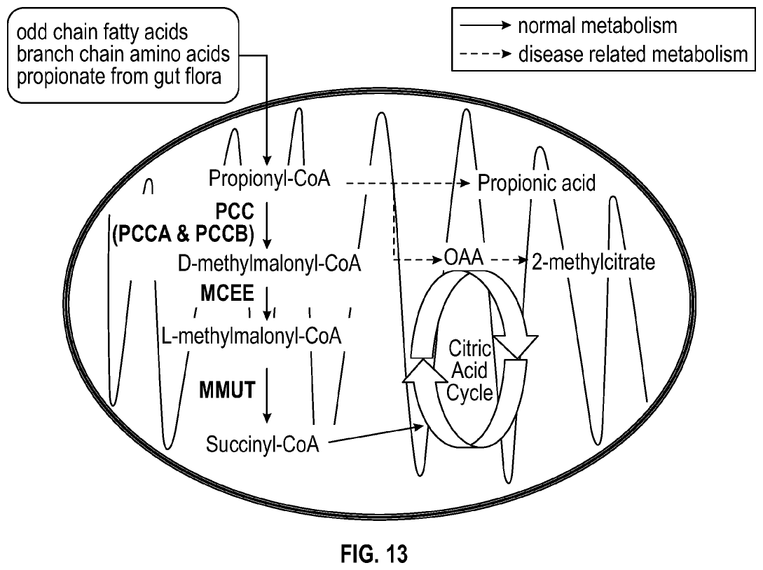

In one aspect, a polypeptide encoded by the polynucleotide expression cassette retains at least 90% of the naturally occurring human PCC protein function, i.e., the capacity to catalyze the carboxylation of propionyl-CoA to D-methylmalonyl-CoA. In another aspect, the encoded PCC protein retains at least 95% of the naturally occurring human PCC protein function. This protein function can be measured, for example, via the efficacy to rescue a neonatal lethal phenotype in Pcca knock-out mice (FIGS. 4, 10) and/or the lowering of circulating metabolites including 2-methylcitrate in a disease model of PA (FIG. 5).

In some aspects, the synPCCA polynucleotide exhibits improved expression relative to the expression of naturally occurring human propionyl-CoA carboxylase alpha polynucleotide sequence. The improved expression is due to the polynucleotide comprising codons that have been optimized relative to the naturally occurring human propionyl-CoA carboxylase alpha polynucleotide sequence. In one aspect, the synthetic polynucleotide has at least 25%, at least 30%, at least 35%, at least 40%, at least 45%, at least 50%, at least 55%, at least 60%, at least 65%, at least 70%, at least 75%, at least 80% of less commonly used codons replaced with more commonly used codons. In additional aspects, the polynucleotide has at least 85%, 90%, or 95% replacement of less commonly used codons with more commonly used codons, and demonstrate equivalent or enhanced expression of PCCA as compared to SEQ ID NO:1.

In some aspects, the synPCCA polynucleotide preferably encodes a polypeptide that retains at least 80% of the enhanced PCC expression (as demonstrated by expression of the polynucleotide of SEQ ID NO:1 in an appropriate host.) In additional aspects, the polypeptide retains at least 85%, 90%, or 95% or 100% of the enhanced expression observed with the polynucleotides of SEQ ID NOs: 2-7.

In designing the synPCCA, the following considerations were balanced. For example, fewer changes to the nucleotide sequence of SEQ ID NO:1 may decrease the potential of altering the secondary structure of the sequence, which can have a significant impact on gene expression. The introduction of undesirable restriction sites is also reduced, which may facilitate the subcloning of PCCA into the plasmid expression vector. However, a greater number of changes to the nucleotide sequence of SEQ ID NO:1 may allow for more convenient identification of the translated and expressed message, e.g. mRNA, in vivo. Additionally, a greater number of changes to the nucleotide sequence of SEQ ID NO:1 may increase the likelihood of greater expression. These considerations were balanced when arriving at SEQ ID NOs: 2-7. The polynucleotide sequences encoding synPCCA may allow for increased expression of the synPCCA gene relative to naturally occurring human PCCA sequences. They are also engineered to have increased transcriptional, translational, and protein refolding efficacy. This engineering is accomplished by using human codon biases, evaluating GC, CpG, and negative GpC content, optimizing the interaction between the codon and anti-codon, and eliminating cryptic splicing sites and RNA instability motifs. The synPCCA polynucleotides may facilitate detection using nucleic acid-based assays.

PCCA has a total of 728 amino acids and synPCCA contains 728 codons corresponding to said amino acids. In SEQ ID NOs: 2-7, codons were changed from that of the natural human PCCA. However, despite changes from SEQ ID NO:1, SEQ ID NOs: 2-7 encode the amino acid sequence SEQ ID NO:8 of PCCA. Codons for SEQ ID NOs: 2-7 are changed, in accordance with the equivalent amino acid positions of SEQ ID NO:8, as seen in Table 2. In this aspect, the amino acid sequence for natural human PCCA has been retained.

It can be appreciated that partial reversion of the designed synPCCA to codons that are found in PCCA can be expected to result in nucleic acid sequences that, when incorporated into appropriate vectors, can also exhibit the desirable properties of SEQ ID NOs: 2-7. For example, such partial reversion or hybrid variants can have PCCA expression from a vector inserted into an appropriate host that is equivalent to that of SEQ ID NOs: 2-7. For example, aspects of the invention include nucleic acids in which at least 1 altered codon, at least 2 altered codons, at least 3 altered codons, at least 4 altered codons, at least 5 altered codons, at least 6 altered codons, at least 7 altered codons, at least 8 altered codons, at least 9 altered codons, at least 10 altered codons, at least 11 altered codons, at least 12 altered codons, at least 13 altered codons, at least 14 altered codons, at least 15 altered codons, at least 16 altered codons, at least 17 altered codons, at least 18 altered codons, at least 20 altered codons, at least 25 altered codons, at least 30 altered codons, at least 35 altered codons, at least 40 altered codons, at least 50 altered codons, at least 55 altered codons, at least 60 altered codons, at least 65 altered codons, at least 70 altered codons, at least 75 altered codons, at least 80 altered codons, at least 85 altered codons, at least 90 altered codons, at least 95 altered codons, at least 100 altered codons, at least 110 altered codons, at least 120 altered codons, at least 130 altered codons, at least 130 altered codons, at least 140 altered codons, at least 150 altered codons, at least 160 altered codons, at least 170 altered codons, at least 180 altered codons, at least 190 altered codons, at least 200 altered codons, at least 220 altered codons, at least 240 altered codons, at least 260 altered codons, at least 280 altered codons, at least 300 altered codons, at least 320 altered codons, at least 340 altered codons, at least 360 altered codons, at least 380 altered codons, at least 400 altered codons, at least 420 altered codons, at least 440 altered codons, at least 460 altered codons, or at least 480 altered codons in SEQ ID NOs: 2-7 are reverted to native codons according to SEQ ID NO:1, and having expression equivalent to that of SEQ ID NO:1. Alternately, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100% of the altered codons in SEQ ID NOs:2-7 are reverted to the native sequence according to SEQ ID NO:1, and having expression that is equivalent to that of SEQ ID NOs: 2-7.

In some aspects, the polynucleotide expression cassettes do not comprise a polynucleotide that shares 100% identity with SEQ ID NO:1. In other words, in some aspects, polynucleotides having 100% identity with SEQ ID NO:1 are excluded from the aspects of the present invention.

The synthetic polynucleotide can be composed of DNA and/or RNA or a modified nucleic acid, such as a peptide nucleic acid, and could be conjugated for improved biological properties.

In one aspect, the polynucleotide expression cassette comprises a synPCCA polynucleotide selected from the group consisting of: (a) a polynucleotide comprising the nucleic acid sequence of any one of SEQ ID NOs:2-7; (b) a polynucleotide having the nucleic acid sequence of any one of SEQ ID NOs:2-7; (c) a polynucleotide having a nucleic acid sequence with at least 80% identity to the nucleic acid sequence of any one of SEQ ID NOs:2-7; (d) a polynucleotide encoding a polypeptide having the amino acid sequence of SEQ ID NO:8 or an amino acid sequence substantially identical to the amino acid sequence of SEQ ID NO:8, wherein the polynucleotide does not have the nucleic acid sequence of SEQ ID NO:1; and (e) a polynucleotide encoding an active fragment of the PCC protein, wherein the polynucleotide in its entirety does not share 100% identity with a portion of the nucleic acid sequence of SEQ ID NO:1.

The phrase “substantially identical”, as used herein, refers to an amino acid sequence exhibiting high identity with a reference amino acid sequence (for example, at least 80%, at least 85%, at least 90%, at least 91%, at least 92%, at least 93%, at least 94%, at least 95%, at least 98%, or at least 99%) and retaining the biological activity of interest (the enzyme activity).

In one aspect, the synPCCA polynucleotide is selected from the group consisting of: a polynucleotide comprising the nucleic acid sequence of any one of SEQ ID NOs: 2-7; a polynucleotide comprising a nucleic acid sequence with at least 80% identity to the nucleic acid sequence of any one of SEQ ID NOs: 2-7 which encodes the polypeptide of SEQ ID NO:8 and has equivalent or greater expression in a host relative to expression of any one of SEQ ID NOs: 2-7 or SEQ ID NO:1, wherein the polynucleotide does not have the nucleic acid sequence of SEQ ID NO:1. In one aspect, the synthetic polynucleotide has at least 90%, 91%, 92%, 93%, 94%, 95%, 98%, or 99% identity to the nucleic acid sequence of any one of SEQ ID NOs:2-7. Another example of a synthetic polynucleotide comprises the nucleotide sequence of SEQ ID NO: 43 (truncated PCCA) or a nucleotide sequence having at least 90%, 91%, 92%, 93%, 94%, 95%, 98%, or 99% identity to the nucleic acid sequence of SEQ ID NO 43.

The fragment may include only amino acid residues encoded by synPCCA, which represents the active, processed form of PCC alpha. By active can be meant, for example, the enzyme's ability to catalyze the carboxylation of propionyl CoA to D-methylmalonyl CoA. The activity can be assayed using methods and assays well-known in the art (as described in the context of protein function, below).

The synPCCA polynucleotide encodes a polypeptide having the amino acid sequence of SEQ ID NO:8 or an amino acid sequence with at least 90%, at least 91%, at least 92%, at least 93%, at least 94%, at least 95%, at least 98%, or at least 99% identity to the amino acid sequence of SEQ ID NO:8.

The synPCCA polynucleotide may exhibit augmented expression relative to the expression of naturally occurring human propionyl-CoA carboxylase alpha polynucleotide sequence (SEQ ID NO:1) in a subject. The synPCCA polynucleotide with augmented expression may comprise a nucleic acid sequence comprising codons that have been optimized relative to the naturally occurring human propionyl-CoA carboxylase alpha polynucleotide sequence (SEQ ID NO:1). The synPCCA polynucleotide may have at least 80% of less commonly used codons replaced with more commonly used codons.

The synPCCA polynucleotide may have a nucleic acid sequence with at least 85% identity to the nucleic acid sequence of any one of SEQ ID NOs: 2-7. In other aspects, the synPCCA polynucleotide has a nucleic acid sequence with at least 90%, at least 91%, at least 92%, at least 93%, at least 94%, at least 95%, at least 98%, at least 99%, or 100% identity to the nucleic acid sequence of any one of SEQ ID NOs: 2-7.

The synPCCA polynucleotide may be a DNA sequence. The synPCCA polynucleotide may be a RNA sequence or peptide modified nucleic acid sequence. The synPCCA polynucleotide may encode an active PCC alpha fragment.

In another aspect, the invention is directed to a recombinant expression vector comprising the herein-described polynucleotide expression cassette. In another aspect of a vector according to the invention, the synPCCA polynucleotide is operably linked to an expression control sequence. In still another aspect, the synPCCA polynucleotide is codon-optimized.

In one aspect, the recombinant expression vector comprising the polynucleotide expression cassette is an AAV vector containing the chicken-beta actin promoter (SEQ ID NO:9), the EF1AL promoter (SEQ ID NO:10), the elongation factor 1 alpha short promoter with a 3′ HPRE (SEQ ID NO: 11), or the short elongation factor 1 alpha promoter with a mutant 3′ HPRE (SEQ ID NO:12).

In another aspect, the recombinant expression vector comprising the polynucleotide expression cassette is an AAV vector containing a liver specific enhancer and promoter, such as the long (SEQ ID NO:14) or short variants (SEQ ID NO:13) of the apolipoprotein E enhancer, operably linked to the long (SEQ ID NO:16) or short variants of the human alpha 1 antitrypsin promoter (SEQ ID NO:15) and followed by either a chimeric intron (SEQ ID NO: 17), modified beta (β)-globin intron (SEQ ID NO: 18), or a synthetic intron (SEQ ID NO:19).

In one aspect, the apolipoprotein E enhancer, and human alpha 1 antitrypsin promoter are operably linked to form a short (SEQ ID NO: 20) or long liver specific enhancer-promoter unit (SEQ ID NO: 21) and placed 5′ to an intron selected from SEQ ID NO: 17-19. In one aspect, the intron is the modified β-globin intron (SEQ ID NO: 18).

In a further aspect, the enhanced human alpha 1 antitrypsin enhancer, promoter, and intron comprises SEQ ID:22.

In another aspect, the liver specific enhancer is derived from sequences upstream of the alpha-1-microglobulin/bikunin precursor (SEQ ID:23 and SEQ ID:24), operably linked to the human thyroxine-binding globulin promoter (TBG) (SEQ ID:25).

In one aspect, the liver specific enhancer and human thyroxine-binding globulin promoter is SEQ ID:26.

The polynucleotide expression cassette of the disclosure can include additional features. For example, the synPCCA polynucleotide can be flanked by a 5′ untranslated region (5′UTR) that includes a strong Kozak translational initiation signal. A 5′UTR can comprise a heterologous polynucleotide fragment and a then a second, third or fourth polynucleotide fragment from the same and/or different UTRs.

In some aspects, the polynucleotide expression cassette of the disclosure comprises an internal ribosome entry site (RES) (SEQ ID: 27) instead of, or in addition to, a UTR.

In one aspect, the UTR can also include at least one translation enhancer element (TEE). A TEE comprises nucleic acid sequences that increase the amount of polypeptide or protein produced from a polynucleotide. As a non-limiting example, the TEE can be located between the promoter and the start codon. In some aspects, the 5′UTR comprises a TEE.

In one aspect, the 5′UTR sequence(s) are derived from genes well known to be highly expressed in the liver. Non-limiting examples include polynucleotides derived from human albumin (SEQ ID: 28), SERPINA 1 (SEQ ID: 29), or SERPINA 3 (SEQ ID: 30).

In one aspect, the polynucleotide expression cassettes of the disclosure include additional features, including the incorporation of sequences designed to stabilize the synthetic PCCA mRNA. In one example, the polynucleotide expression cassette comprises the wood chuck post-translational response element (SEQ ID: 31) In another non-limiting example, the polynucleotide expression cassette comprises the hepatitis post-translational response element (SEQ ID:32).

In one aspect, the polynucleotide expression cassette contains a synPCCA polynucleotide including a polyadenylation signal, such as that derived from the rabbit beta globin gene or the bovine growth hormone gene. Such sequences are well known to practitioners of the art.

In one aspect, terminal repeat sequences (SEQ ID:33-34) from the piggyBac transposon, which is originally isolated from the cabbage looper (Trichoplusia ni; a moth species), are inserted immediately after the 5′AAV ITR and before the 3′ AAV ITR. piggyBac is a class II transposon, moving in a cut-and-paste manner. An AAV vector that contains piggyBac terminal repeat sequences can serve as a substrate for piggyBac transposase, which, when introduced by a viral or non-viral vector, can mediate the permanent integration of the AAV polynucleotide expression cassette containing the synPCCA polynucleotide into the transduced cell. Hybrid AAV-piggyBac transposon vectors are well understood by practitioners of the art, and can be used to deliver the synPCCA polynucleotide to a target cell in vitro and in vivo.

One aspect of a AAV recombinant expression vector comprising a polynucleotide expression cassette designed to express synPCCA1 incorporates the enhanced TBG promoter of SEQ ID:35.

In one aspect, a AAV recombinant expression vector comprising a polynucleotide expression cassette designed to express synPCCA1 incorporates the enhanced human alpha 1 antitrypsin promoter of SEQ ID:36.

In one aspect, the polynucleotide expression cassettes are configured to integrate into the human albumin locus. A donor cassette is constructed that targets the stop codon of human albumin, which yields, after homologous recombination, synPCCA1 that is fused via a P2 peptide to the carboxy terminus of albumin.

In one aspect, the vector that is an integrating AAV vector, from 5′ITR to 3′ITR, that uses homologous recombination to insert the polynucleotide expression cassette into the end of Albumin, which is a safe harbor for gene editing, is SEQ ID:37.

In one aspect, the integrating AAV vector, from 5 ITR to 3′ITR, that uses homologous recombination to insert the polynucleotide expression cassette into 5′ end of Albumin is SEQ ID:38.

In one aspect, the polynucleotide expression cassettes of this application are configured to integrate into the genome after delivery using a lentiviral vector.

In one aspect, a lentiviral vector is designed to express the polynucleotide expression cassette using an enhanced human alpha 1 antitrypsin enhancer and promoter of SEQ ID:39.

In yet another aspect, a lentiviral vector designed to express the polynucleotide expression cassette using the elongation factor 1 long promoter is SEQ ID:40.

In an aspect of the invention, the polynucleotide expression cassette further comprises any one or more of the following features: (i) a 5′ ITR nucleotide sequence at least 90%, 91%, 92%, 93%, 94%, 95%, 98%, 99%, or 100% identical to nucleotides 1 to 130 of SEQ ID NO: 41; (ii) a core elongation factor 1 alpha (EF1S) promoter nucleotide sequence at least 90%, 91%, 92%, 93%, 94%, 95%, 98%, 99%, or 100% identical to nucleotides 131 to 361 of SEQ ID NO: 41; (iii) an intron nucleotide sequence at least 90%, 91%, 92%, 93%, 94%, 95%, 98%, 99%, or 100% identical to nucleotides 362 to 526 of SEQ ID NO: 41; (iv) a HPRE nucleotide sequence at least 90%, 91%, 92%, 93%, 94%, 95%, 98%, 99%, or 100% identical to nucleotides 2714 to 3439 of SEQ ID NO: 41; (v) a polyA nucleotide sequence at least 90%, 91%, 92%, 93%, 94%, 95%, 98%, 99%, or 100% identical to nucleotides 3440 to 3664 of SEQ ID NO: 41; and (vi) a 3′ ITR nucleotide sequence at least 90%, 91%, 92%, 93%, 94%, 95%, 98%, 99%, or 100% identical to nucleotides 3665 to 3805 of SEQ ID NO: 41.

In an aspect of the invention, the polynucleotide expression cassette further comprises all of the following features: (i) a 5′ ITR nucleotide sequence at least 90%, 91%, 92%, 93%, 94%, 95%, 98%, 99%, or 100% identical to nucleotides 1 to 130 of SEQ ID NO: 41; (ii) a EF1S promoter nucleotide sequence at least 90%, 91%, 92%, 93%, 94%, 95%, 98%, 99%, or 100% identical to nucleotides 131 to 361 of SEQ ID NO: 41; (iii) an intron nucleotide sequence at least 90%, 91%, 92%, 93%, 94%, 95%, 98%, 99%, or 100% identical to nucleotides 362 to 526 of SEQ ID NO: 41; (iv) a HPRE nucleotide sequence at least 90%, 91%, 92%, 93%, 94%, 95%, 98%, 99%, or 100% identical to nucleotides 2714 to 3439 of SEQ ID NO: 41; (v) a polyA nucleotide sequence at least 90%, 91%, 92%, 93%, 94%, 95%, 98%, 99%, or 100% identical to nucleotides 3440 to 3664 of SEQ ID NO: 41; and (vi) a 3′ ITR nucleotide sequence at least 90%, 91%, 92%, 93%, 94%, 95%, 98%, 99%, or 100% identical to nucleotides 3665 to 3805 of SEQ ID NO: 41.

In an aspect of the invention, the synPCCA polynucleotide comprises a synPCCA1 nucleotide sequence at least 90%, 91%, 92%, 93%, 94%, 95%, 98%, 99%, or 100% identical to SEQ ID NO: 2.

In an aspect of the invention, the polynucleotide expression cassette comprises any one or more of the following features: (i) a 5′ ITR nucleotide sequence consisting of a nucleotide sequence at least 90%, 91%, 92%, 93%, 94%, 95%, 98%, 99%, or 100% identical to nucleotides 1 to 130 of SEQ ID NO: 41; (ii) a EF1S promoter nucleotide sequence consisting of a nucleotide sequence at least 90%, 91%, 92%, 93%, 94%, 95%, 98%, 99%, or 100% identical to nucleotides 131 to 361 of SEQ ID NO: 41; (iii) a intron nucleotide sequence consisting of a nucleotide sequence at least 90%, 91%, 92%, 93%, 94%, 95%, 98%, 99%, or 100% identical to nucleotides 362 to 526 of SEQ ID NO: 41; (iv) a HPRE nucleotide sequence consisting of a nucleotide sequence at least 90%, 91%, 92%, 93%, 94%, 95%, 98%, 99%, or 100% identical to nucleotides 2714 to 3439 of SEQ ID NO: 41; (v) a polyA nucleotide sequence consisting of a nucleotide sequence at least 90%, 91%, 92%, 93%, 94%, 95%, 98%, 99%, or 100% identical to nucleotides 3440 to 3664 of SEQ ID NO: 41; and (vi) a 3′ ITR nucleotide sequence consisting of a nucleotide sequence at least 90%, 91%, 92%, 93%, 94%, 95%, 98%, 99%, or 100% identical to nucleotides 3665 to 3805 of SEQ ID NO: 41.

In an aspect of the invention, the polynucleotide expression cassette comprises all of the following features: (i) a 5′ ITR nucleotide sequence consisting of a nucleotide sequence at least 90%, 91%, 92%, 93%, 94%, 95%, 98%, 99%, or 100% identical to nucleotides 1 to 130 of SEQ ID NO: 41; (ii) a EF1S promoter nucleotide sequence consisting of a nucleotide sequence at least 90%, 91%, 92%, 93%, 94%, 95%, 98%, 99%, or 100% identical to nucleotides 131 to 361 of SEQ ID NO: 41; (iii) a intron nucleotide sequence consisting of a nucleotide sequence at least 90%, 91%, 92%, 93%, 94%, 95%, 98%, 99%, or 100% identical to nucleotides 362 to 526 of SEQ ID NO: 41; (iv) a HPRE nucleotide sequence consisting of a nucleotide sequence at least 90%, 91%, 92%, 93%, 94%, 95%, 98%, 99%, or 100% identical to nucleotides 2714 to 3439 of SEQ ID NO: 41; (v) a polyA nucleotide sequence consisting of a nucleotide sequence at least 90%, 91%, 92%, 93%, 94%, 95%, 98%, 99%, or 100% identical to nucleotides 3440 to 3664 of SEQ ID NO: 41; and (vi) a 3′ ITR nucleotide sequence consisting of a nucleotide sequence at least 90%, 91%, 92%, 93%, 94%, 95%, 98%, 99%, or 100% identical to nucleotides 3665 to 3805 of SEQ ID NO: 41.

In an aspect of the invention, the synPCCA polynucleotide consists of a synPCCA1 nucleotide sequence at least 90%, 91%, 92%, 93%, 94%, 95%, 98%, 99%, or 100% identical to SEQ ID NO: 2.

In an aspect of the invention, the polynucleotide expression cassette comprises a nucleotide sequence at least 90%, 91%, 92%, 93%, 94%, 95%, 98%, 99%, or 100% identical to SEQ ID NO: 41 or SEQ ID NO: 42.

In an aspect of the invention, the polynucleotide expression cassette consists of a nucleotide sequence at least 90%, 91%, 92%, 93%, 94%, 95%. 98%, 99%, or 100% identical to the nucleotide sequence of SEQ ID NO: 41 or SEQ ID NO: 42. Another example of a recombinant expression vector is the nucleotide sequence of SEQ ID NO: 44.

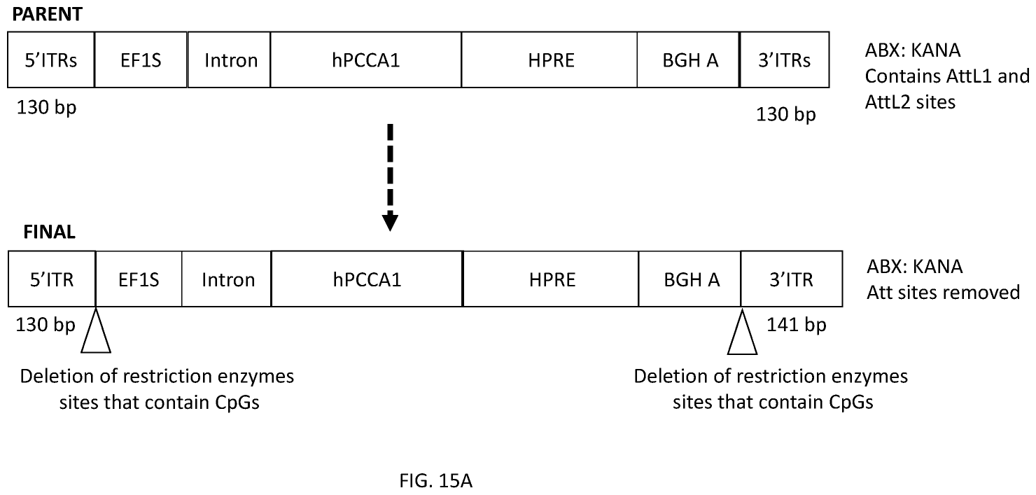

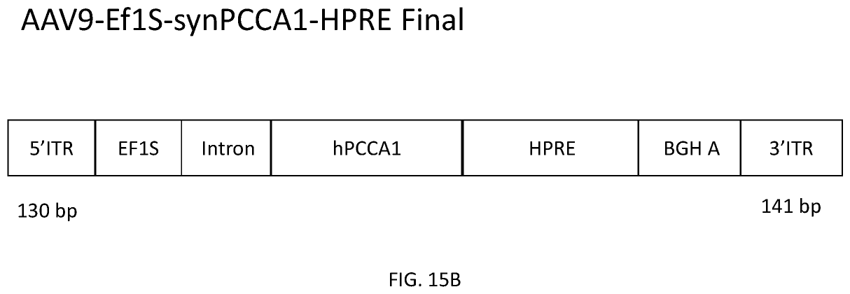

Aspects of the invention may provide recombinant expression vectors comprising any of the inventive polynucleotide expression cassettes described herein. The vector may be a recombinant adeno-associated virus (rAAV). The rAAV may comprise an AAV capsid and a vector genome packaged therein. The vector genome may comprise: a 5′-inverted terminal repeat sequence (5′-ITR) sequence; a promoter sequence; a 5′ untranslated region; a Kozak sequence; a partial fragment or complete coding sequence for PCCA; an mRNA stability sequence; a polyadenylation signal; and a 3′-inverted terminal repeat sequence (3′-ITR) sequence. In one aspect, the rAAV is comprised of the structure in FIG. 15B. In one aspect, the AAV capsid is from an AAV of serotype 1, 2, 3, 4, 5, 6, 7, 8, 9, 10, 11, 12, 13, rh 10, hu37 or Anc, or mutants thereof. In one aspect, the AAV capsid is from an AAV of serotype 8. In one aspect, the AAV capsid is from an AAV of serotype 9. In one aspect, the rAAV further contains terminal repeat sequences recognized by piggyBac transposase internal to the 5′ and 3′ ITR.

### Therapy

Any of the inventive polynucleotide expression cassettes, recombinant expression vectors, rAAVs, or compositions described herein can be used as a drug, via viral- or non-viral mediated gene delivery, to restore PCC function in PA patients, prevent metabolic instability, and ameliorate disease progression. Because this enzyme is involved in other human disorders of branched chain amino acid oxidation, gene delivery of a synthetic PCCA gene might used to treat conditions other than PA.

In another aspect, the invention comprises a method of treating a disease or condition mediated by PCC, comprising administering to a subject in need thereof a therapeutic amount of any of the inventive polynucleotide expression cassettes, recombinant expression vectors, rAAVs, or compositions described herein. The disease or condition can, in one aspect, be PA. The PCC enzyme is processed after transcription, translation, and translocation into the mitochondrial inner space.

Enzyme replacement therapy includes administration of the functional enzyme (PCC) to a subject in a manner so that the enzyme administered will catalyze the reactions in the body that the subject's own defective or deleted enzyme cannot. In enzyme therapy, the defective enzyme can be replaced in vivo or repaired in vitro using the polynucleotide expression cassette according to the invention. The functional enzyme molecule can be isolated or produced in vitro, for example. Methods for producing recombinant enzymes in vitro are known in the art. In vitro enzyme expression systems include, without limitation, cell-based systems (bacterial (for example, Escherichia coli, Corynebacterium, Pseudomonas fluorescens), yeast (for example, Saccharomyces cerevisiae, Pichia Pastoris), insect cell (for example, Baculovirus-infected insect cells, non-lytic insect cell expression), and eukaryotic systems (for example, Leishmania)) and cell-free systems (using purified RNA polymerase, ribosomes, tRNA, ribonucleotides). Viral in vitro expression systems are likewise known in the art. The enzyme isolated or produced according to the above-iterated methods exhibits, in specific aspects, 80%, 85%, 90%, 95%, 98%, 99%, or 100% homology to the naturally occurring (for example, human) PCC.

Gene therapy can involve in vivo gene therapy (direct introduction of the genetic material into the cell or body) or ex vivo gene transfer, which usually involves genetically altering cells prior to administration. In one aspect, genome editing, or genome editing with engineered nucleases (GEEN) may be performed with the polynucleotide expression cassettes of the present invention allowing synPCCA DNA to be inserted, replaced, or removed from a genome using artificially engineered nucleases. Any known engineered nuclease may be used such as ZFNs, TALENs, the CRISPR/Cas system, and engineered meganuclease re-engineered homing endonucleases. Alternately, the nucleotides of the present invention including synPCCA, in combination with a CASP/CRISPR, ZFN, or TALEN can be used to engineer correction at the locus in a patient's cell either in vivo or ex vivo, then, in one aspect, use that corrected cell, such as a fibroblast or lymphoblast, to create an iPS or other stem cell for use in cellular therapy.

In one aspect, the method of treating a disease or condition mediated by PCC in a subject in need thereof, comprising administering to a cell of the subject, or a population of cells of the subject, any of the inventive polynucleotide expression cassettes, recombinant expression vectors, rAAVs, or compositions described herein, wherein the polynucleotide expression cassette is inserted into the cell of the subject, or the population of cells of the subject, via genome editing on the cell of the subject, or the population of cells of the subject, using a nuclease selected from the group of ZFNs, TALENs, the CRISPR/cas system and meganuclease re-engineered homing endonucleases on the cell from the subject, or the population of cells of the subject; and administering the cell, or population of cells, to the subject.

In another aspect, the polynucleotide expression cassettes of the present invention can be used in combination with a non-integrating vector or as naked DNA, and configured to contain terminal repeat sequences for a transposon recognition by a transposase such as piggyBac. The use of hybrid AAV and adenoviral vectors that combine the transient or regulated expression of a transposase like piggyBac may be performed to enable permanent correction by cut and paste transposition. Alternatively, the transposase mRNA, encapsulated as lipid-nanoparticle, might be used to deliver piggBac transposase.

In one aspect, the invention is directed to a method of treating a disease or condition mediated by PCC or low levels of PCC activity, the method comprising administering to a subject any of the inventive polynucleotide expression cassettes, recombinant expression vectors, rAAVs, or compositions described herein.

In one aspect, the invention is directed to a method of treating a disease or condition mediated by PCC, the method comprising administering to a subject a PCC produced using any of the inventive polynucleotide expression cassettes, recombinant expression vectors, rAAVs, or compositions described herein. In another aspect of a method of treatment according to the invention, the disease or condition is PA.

In one aspect, the invention is directed to the preclinical amelioration or rescue from the disease state, for example, propionic acidemia, that the afflicted subject exhibits. This may include symptoms, such as lethargy, lethality, metabolic acidosis, and biochemical perturbations, such as increased levels of methylcitrate in blood, urine, and body fluids.

Additionally, any of the inventive polynucleotide expression cassettes, recombinant expression vectors, rAAVs, or compositions described herein can be used for the in vitro production of PA for use in enzyme replacement therapy for PA. Enzyme replacement therapy may be accomplished by administration of the synthetic PCC protein, sub-cutaneously, intra-muscularly, intravenously, or by other therapeutic delivery routes.

The term “subject”, as used herein, refers to a domesticated animal, a farm animal, a primate, a mammal, for example, a human.

### Administration Delivery and Dosage Forms

Routes of delivery of a synPCCA polynucleotide expression cassette according to the invention may include, without limitation, injection (systemic or at target site), for example, intradermal, subcutaneous, intravenous, intraperitoneal, intraocular, subretinal, renal artery, hepatic vein, intramuscular injection; physical, including ultrasound (-mediated transfection), electric field-induced molecular vibration, electroporation, transfection using laser irradiation, photochemical transfection, gene gun (particle bombardment); parenteral and oral (including inhalation aerosols and the like). Related methods include using genetically modified cells.

In one aspect, the polynucleotide expression cassette, recombinant expression vector, rAAV, or composition is administered subcutaneously, intramuscularly, intradermally, intraperitoneally, or intravenously.

In one aspect, the rAAV is administered at a dose of 1×10¹¹ to 1×10¹⁴ genome copies (GC)/kg.

In one aspect, the administering the rAAV comprises administration of a single dose of rAAV; in one aspect, administering the rAAV comprises administration of multiple doses of rAAV.

Vehicles for delivery of a synthetic propionyl-CoA carboxylase (synPCCA) polynucleotide expression cassette according to the invention may include, without limitation, viral vectors (for example, AAV, integrating AAV vectors, adenovirus, baculovirus, retrovirus, lentivirus, foamy virus, herpes virus, Moloney murine leukemia virus, Vaccinia virus, and hepatitis virus) and non-viral vectors (for example, naked DNA, mini-circles, liposomes, ligand-polylysine-DNA complexes, nanoparticles, including mRNA containing lipid nanoparticles, cationic polymers, including polycationic polymers such as dendrimers, synthetic peptide complexes, artificial chromosomes, and polydispersed polymers). Thus, dosage forms contemplated include injectables, aerosolized particles, capsules, and other oral dosage forms.

In one specific aspect, synPCCA could be placed under the transcriptional control of a ubiquitous or tissue-specific promoter, with a 5′ intron, 5′ intron translational enhancer element, and flanked by an mRNA stability element, such as the woodchuck or hepatitis post-transcriptional regulatory element, and polyadenylation signal. The use of a tissue-specific promoter can restrict unwanted transgene expression, as well as facilitate persistent transgene expression. The therapeutic transgene could then be delivered as coated or naked DNA into the systemic circulation, portal vein, or directly injected into a tissue or organ, such as the liver or kidney. In addition to the liver or kidney, the brain, pancreas, eye, heart, lungs, bone marrow, and muscle may constitute targets for therapy. Other tissues or organs may be additionally contemplated as targets for therapy.

In another aspect, the same polynucleotide expression cassette could be packaged into a viral vector, such as an adenoviral vector, retroviral vector, lentiviral vector, or adeno-associated viral vector, and delivered by various means into the systemic circulation, portal vein, or directly injected into a tissue or organ, such as the liver or kidney. In addition to the liver or kidney, the brain, pancreas, eye, heart, lungs, bone marrow, and muscle may constitute targets for therapy. Other tissues or organs may be additionally contemplated as targets for therapy.

Tissue-specific promoters include, without limitation, Apo A-I, ApoE, hAAT, transthyretin, liver-enriched activator, albumin, TBG, PEPCK, and RNAP_{II} promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, dβH, prolactin, CaMK2, and myelin basic protein promoters (neuronal), and ankyrin, α-spectrin, globin, HLA-DRα, CD4, glucose 6-phosphatase, and dectin-2 promoters (erythroid).

Regulatable promoters (for example, ligand-inducible or stimulus-inducible promoters) and optogenetic promoters are also contemplated for expression constructs according to aspects of the invention.

In yet another aspect, the polynucleotide expression cassette could be used in ex vivo applications via packaging into a retro- or lentiviral vector to create an integrating vector that could be used to permanently correct any cell type from a patient with PCC deficiency. The synPCCA-transduced and corrected cells could then be used as a cellular therapy. Examples might include CD34+ stem cells, primary hepatocytes, or fibroblasts derived from patients with PCC deficiency. Fibroblasts could be reprogrammed to other cell types using iPS methods well known to practitioners of the art. In yet another aspect, the polynucleotide expression cassette could be recombined using genomic engineering techniques that are well known to practitioners of the art, such as ZFNs and TALENS, into the PCCA locus, a genomic safe harbor site, such as AAVS1, or into another advantageous location, such as into rDNA, the albumin locus, GAPDH, or a suitable expressed pseudogene. In yet another aspect, synPCCA could be delivered using a hybrid AAV-piggyBac transposon system as is well known to practitioners of the art (see PMID: 31099022), and references therein: Siew et al., Hepatology, 70(6): 2047-2061 (2019).

Aspects of the invention may further provide compositions comprising (a) any of the inventive polynucleotide expression cassettes, recombinant expression vectors, or rAAVs described herein, and (b) a pharmaceutically acceptable carrier. In one aspect, the composition further comprises a hybrid AAV-piggyBac transposon system.

A composition (pharmaceutical composition) for treating an individual by gene therapy may comprise a therapeutically effective amount of a vector comprising the synPCCA transgenes or a viral particle produced by or obtained from same. The pharmaceutical composition may be for human or animal usage. Typically, a physician will determine the actual dosage which will be most suitable for an individual subject, and it will vary with the age, weight, and response of the particular individual.

The composition may, in specific aspects, comprise a pharmaceutically acceptable carrier, diluent, excipient, or adjuvant. Such materials should be non-toxic and should not interfere with the efficacy of the transgene. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington: The Science and Practice of Pharmacy, 22^{nd} Ed., Pharmaceutical Press (2012). The choice of pharmaceutical carrier, excipient, or diluent can be selected with regard to the intended route of administration and standard pharmaceutical practice. The pharmaceutical compositions may comprise as, or in addition to, the carrier, excipient, or diluent any suitable binder(s), lubricant(s), suspending agent(s), coating agent(s), solubilizing agent(s), and other carrier agents that may aid or increase the viral entry into the target site (such as for example a lipid delivery system). For oral administration, excipients such as starch or lactose may be used. Flavoring or coloring agents may be included, as well. For parenteral administration, a sterile aqueous solution may be used, optionally containing other substances, such as salts or monosaccharides to make the solution isotonic with blood.

As used herein, a “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In certain aspects, it may be preferable to include isotonic agents, for example, sugars, polyalcohols such as mannitol, sorbitol, or sodium chloride in the composition.

A composition according to the invention may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, modulators, or drugs (e.g., antibiotics).

The composition may be in a variety of forms. These include, for example, liquid, semi-solid and solid dosage forms, such as liquid solutions (e.g., injectable and infusible solutions), dispersions or suspensions, tablets, pills, powders, liposomes and suppositories. Additional dosage forms contemplated include: in the form of a suppository or pessary; in the form of a lotion, solution, cream, ointment or dusting powder; by use of a skin patch; in capsules or ovules; in the form of elixirs, solutions, or suspensions; in the form of tablets or lozenges.

A “therapeutically effective amount” refers to an amount effective, at dosages and for periods of time necessary, to achieve the desired therapeutic result. A therapeutically effective amount of a vector comprising the polynucleotide expression cassette of the invention may vary according to factors such as the disease state, age, sex, and weight of the individual, and the ability of the vector to elicit a desired response in the individual. A therapeutically effective amount is also one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of the polynucleotide expression cassette or a fragment thereof according to the invention calculated to produce the desired therapeutic effect in association with a pharmaceutical carrier.

### Primers, Probes, and Methods of Detection

Nucleic acids consisting of portions of the synPCCA polynucleotides (and nucleic acids comprising nucleotide sequences complementary thereto) may facilitate detection using nucleic acid-based assays. Accordingly, nucleic acids consisting of portions of the synPCCA polynucleotides (and nucleic acids comprising nucleotide sequences complementary thereto) described herein may provide forward primers, reverse primers, and probes which may, advantageously, specifically hybridize with nucleic acid from cells for detection of the expression of synPCCA nucleic acid by the cells. In some aspects, these synPCCA nucleic acids specifically hybridize with nucleic acid from cells. The synPCCA nucleic acids of the invention may provide many advantages. These advantages may include, for example, the rapid, sensitive, and specific detection of the expression of synPCCA nucleic acid by cells.

An aspect of the invention provides a synPCCA nucleic acid consisting of (a) a nucleotide sequence that is at least 12 but no more than 35 contiguous nucleotides of any one of SEQ ID NO: 2-7; (b) a nucleotide sequence that is at least 90% identical to at least 12 but no more than 35 contiguous nucleotides of any one of SEQ ID NO: 2-7; or (c) a nucleotide sequence that is complementary to (a) or (b).

In an aspect of the invention, the synPCCA nucleic acid consists of:
(a) a nucleotide sequence that is at least 12 but no more than 34, 33, 32, 31, 30, 29, 28, 27, 26, 25, 24, 23, 22, 21, 20, 19, 18, 17, 16, 15, 14, 13, or 12 contiguous nucleotides of any one of SEQ ID NO: 2-7;
(b) a nucleotide sequence that is at least 12, 13, 14, 15, 16, 17, 18, 19, 20, 21, 22, 23, 24, 25, 26, 27, 28, 29, 30, 31, 32, 33, or 34 but no more than 35 contiguous nucleotides of any one of SEQ ID NO: 2-7;
(c) a nucleotide sequence that is at least 90%, 91%, 92%, 93%, 94%, 95%, 98%, or 99% identical to at least 12 but no more than 4, 33, 32, 31, 30, 29, 28, 27, 26, 25, 24, 23, 22, 21, 20, 19, 18, 17, 16, 15, 14, 13, or 12 contiguous nucleotides of any one of SEQ ID NO: 2-7;
(d) a nucleotide sequence that is at least 90%, 91%, 92%, 93%, 94%, 95%, 98%, or 99% identical to at least 12, 13, 14, 15, 16, 17, 18, 19, 20, 21, 22, 23, 24, 25, 26, 27, 28, 29, 30, 31, 32, 33, or 34 but no more than 35 contiguous nucleotides of any one of SEQ ID NO: 2-7; or
(e) a nucleotide sequence that is complementary to (a), (b), (c), or (d).

In an aspect of the invention, the synPCCA nucleic acid (e.g., probe) further comprises a detectable label. The label may be any label suitable for detecting hybridization, e.g., a complex, of the synPCCA nucleic acid (e.g., probe) with nucleic acid from cells of a subject. Exemplary detectable labels may include any one or more of radioactive labels, non-radioactive labels, fluorescent labels, and chemiluminescent labels.

Another aspect of the invention provides a collection of synPCCA nucleic acids comprising two or more of any of the synPCCA nucleic acids described herein. In an aspect of the invention, the collection may comprise or further comprise a nucleotide sequence complementary to any of the synPCCA nucleic acids described herein. The collection may comprise any suitable number of inventive synPCCA nucleic acids. For example, the collection may comprise from about 2 to about 75 or more synPCCA nucleic acids, from about 10 or less to about 70 or more synPCCA nucleic acids, from about 20 or less to about 60 or more synPCCA nucleic acids, or from about 30 or less to about 50 or more synPCCA nucleic acids. In this regard, the collection may comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, or 74 or more synPCCA nucleic acids. Although the two or more synPCCA nucleic acids of the collection may be identical to one another, in a preferred aspect, the two or more synPCCA nucleic acids are different from each other.

In an aspect of the invention, the collection of synPCCA nucleic acids includes a first synPCCA nucleic acid and a second synPCCA nucleic acid. The first synPCCA nucleic acid may be a forward primer and the second synPCCA nucleic acid may be a reverse primer described herein.

In an aspect of the invention, the collection of synPCCA nucleic acids may include at least one primer and a probe, preferably at least one forward primer, at least one reverse primer, and at least one probe. The probe may comprise any of the synPCCA nucleic acids described herein with respect to other aspects of the invention.

An aspect of the invention provides methods for detecting the expression of synPCCA nucleic acid by cells. These methods may be useful for any of a variety of applications including, for example, any one or more of testing, detecting, quantifying and monitoring the expression of synPCCA polynucleotides by cells after any of the polynucleotide expression cassettes, recombinant expression vectors, rAAVs, or compositions have been administered to the cells. For example, the expression of synPCCA nucleic acid by cells may be tested before administering synPCCA nucleic acid to a subject. If a vector comprising the synPCCA nucleic acid is manufactured, the inventive methods for detecting the expression of synPCCA nucleic acid by cells can be used to measure relative expression of PCCA from the vector. In an aspect of the invention, the method for detecting the expression of synPCCA nucleic acid by cells is carried out by RNA or DNA in situ hybridization.

In an aspect, the method for detecting the expression of synPCCA nucleic acid comprises administering to cells any of the inventive polynucleotide expression cassettes, recombinant expression vectors, rAAVs, or compositions described herein and isolating a sample comprising nucleic acid from the cells that have been administered any of the inventive polynucleotide expression cassettes, recombinant expression vectors, rAAVs, or compositions described herein.

In an aspect of the invention, the method for detecting the expression of synPCCA nucleic acid is carried out prior to administering any of the inventive polynucleotide expression cassettes, recombinant expression vectors, rAAVs, or compositions described herein to a subject.

In an aspect, the method for detecting the expression of synPCCA nucleic acid comprises contacting a sample comprising nucleic acid from cells with any of the inventive synPCCA nucleic acids (primers or probes) or collections of synPCCA nucleic acids (primers or probes) described herein.

The method may comprise extracting nucleic acid from the cells in any suitable manner as is known in the art. The nucleic acid may be RNA and/or DNA. The protocol for extracting nucleic acid may be selected depending on the subject, type of cells in the sample, and nucleic acid to be tested as is known in the art. Preferably, the nucleic acid is extracted in any manner which recovers a testable amount of nucleic acid. The nucleic acid extraction may be carried out using any of a variety of commercially available nucleic acid extraction kits.

The method may comprise contacting the sample of extracted nucleic acid with the synPCCA nucleic acids under conditions which allow the synPCCA nucleic acids to specifically hybridize with the extracted nucleic acid as is known in the art. The method may comprise amplifying the synPCCA nucleic acids and the extracted nucleic acid using any suitable amplification method as is known in the art (e.g., reverse transcription PCR (RT-PCR), or other suitable technique).

The method may further comprise detecting the complex, wherein detection of the complex is indicative of the expression of synPCCA polynucleotide by the cells. Detection of the complex can occur through any number of ways known in the art. In an aspect, the method comprises measuring light emitted from a fluorescent dye using, e.g., a laser. Detecting the complex may, optionally, further comprise measuring the amount of complex formed.

In an aspect of the invention, the method further comprises comparing a presence of the complex in the sample with an absence of complex from a negative sample that lacks synPCCA nucleic acid, wherein detection of the complex is indicative of the expression of synPCCA nucleic acid by the cells.

In an aspect, the inventive methods of detecting the expression of synPCCA nucleic acid by cells may be useful for testing, detecting, quantifying and monitoring the expression of synPCCA polynucleotides by cells of a subject after any of the polynucleotide expression cassettes, recombinant expression vectors, rAAVs, or compositions have been administered to the subject.

In an aspect, the cells are from a subject that has been treated with any of the inventive polynucleotide expression cassettes, recombinant expression vectors, rAAVs, or compositions described herein, and detection of the complex is indicative of the expression of synPCCA nucleic acid by the cells of the subject.

The contacting of the nucleic acid sample from the cells with the synPCCA nucleic acid (or collection of synPCCA nucleic acids) can take place in vitro or in vivo with respect to the subject. Preferably, the contacting is in vitro.

With respect to the inventive methods, the cells may be any type of cells. In an aspect, the cells are heart cells or liver cells. Examples of liver cells include hepatocytes (HCs), hepatic stellate cells (HSCs), Kupffer cells (KCs), and liver sinusoidal endothelial cells (LSECs). Examples of heart cells include cardiomyocytes (CMs), fibroblasts (FBs), endothelial cells (ECs), and peri-vascular cells. In a preferred aspect, the cells are hepatocytes or cardiomyocytes.

When the cells are not cells from a subject, for example, when the method is carried out prior to administering any of the inventive polynucleotide expression cassettes, recombinant expression vectors, rAAVs, or compositions described herein to a subject, the cells may be cells of a cell line. In some aspects, the cells are cells of a heart cell line or a liver cell line. In some preferred aspects, the cells are cells of a hepatocyte cell line or a cardiomyocyte cell line.

In some aspects, the cells are cells of a hepatocyte cell line which does not express one or both of endogenous PCCA mRNA and PCCA protein. For example, the hepatocyte cell line may be a hepatocyte PCCA knock out (KO) cell line (e.g., a HEPG2 PCCA knockout cell line). Cell lines which do not express one or both of endogenous PCCA mRNA and PCCA protein may provide any one or more of a variety of advantages. For example, such cells may make it possible to test, detect, quantify and/or monitor synPCCA nucleic acid expression without possible cellular effects (e.g., a KO cell line may provide little or no background signal that may interfere with the detection of the synPCCA nucleic acid expression).

In another aspect of the invention, the inventive methods of detecting the expression of synPCCA nucleic acid by cells further comprise quantifying the synPCCA nucleic acid expressed by the cells. The synPCCA nucleic acid expressed by the cells may be quantified by any suitable technique known in the art. For example, the quantity of synPCCA nucleic acid expressed by the cells may be compared to a known quantity of expression of a different, reference gene (e.g., Gapdh or beta actin), or the quantity of synPCCA nucleic acid expressed by the cells may be normalized to the quantity of expression of a different, endogenously expressed gene (e.g., Gapdh or beta actin).

In another aspect of the invention, the inventive methods of detecting the expression of synPCCA nucleic acid by cells further comprise quantifying the cells that express the synPCCA nucleic acid. The cells that express the synPCCA nucleic acid may be quantified by any suitable technique known in the art. For example, the quantity of cells expressing synPCCA nucleic acid may be compared to a known quantity of cells expressing a different, reference gene (e.g., Gapdh or beta actin), or the quantity of cells expressing synPCCA nucleic acid may be normalized to the quantity of cells expressing a different, endogenously expressed gene (e.g., Gapdh or beta actin).

As used herein, “determining”, “determination”, “detecting”, or the like are used interchangeably herein and refer to the detecting or quantitation (measurement) of a molecule using any suitable method, including immunohistochemistry, fluorescence, chemiluminescence, radioactive labeling, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like. “Detecting” and its variations refer to the identification or observation of the presence of a molecule in a biological sample, and/or to the measurement of the molecule's value.

### EXAMPLES

### Example 1

This example demonstrates that synPCCA1 is expressed at a higher level than the wild type human PCCA1 gene in cell culture studies.

Six synthetic codon-optimized human propionyl-CoA carboxylase subunit alpha genes (synPCCA1-6) were engineered using an iterative approach, wherein the naturally occurring PCCA cDNA (NCBI Reference Sequence: NM_000282.4) was optimized codon by codon to create (synPCCA1-6) (SEQ ID NOs: 2-7), using a variety of codon optimization methods, one of which incorporated factors involved in protein expression, such as codon adaptability, mRNA structure, and various cis-elements in transcription and translation. The resulting sequences were manually inspected and subject to expert adjustment. The synPCCA alleles displayed maximal divergence from the PCCA cDNA at the nucleotide level yet retained optimally utilized codons at each position.

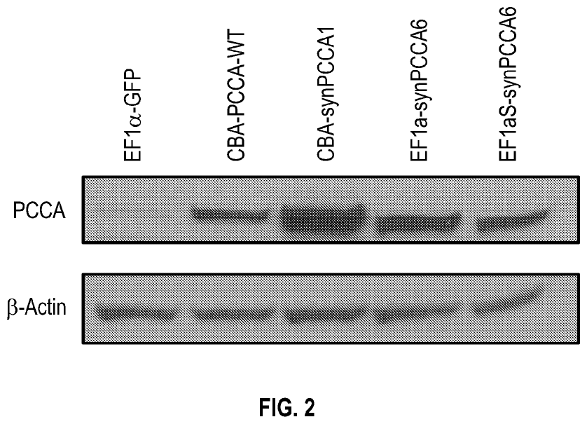

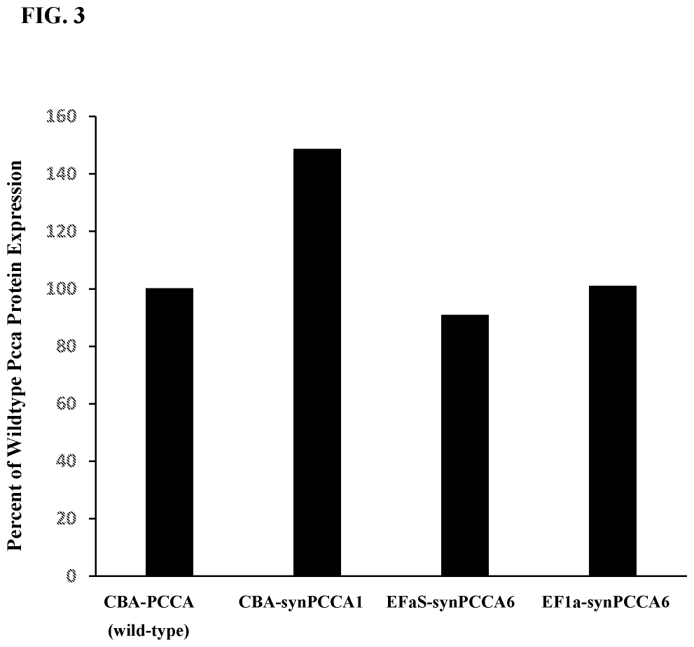

To improve the expression of PCC and create a vector that could express the human PCCA gene in a more efficient fashion, synPCCA1 was cloned using restriction endonuclease excision and DNA ligation into an expression vector under the control of the strong chicken R-actin promoter (CBA) (Chandler, et al. 2010 Mol Ther 18:11-6) or the active but not as potent elongation factor 1 alpha promoter (EF1a). The constructs expressing either PCCA or synPCCA1 with the CBA or synPCCA6 with EF1α long or short promoters were then transfected into 293FT cells using Lipofectamine™ (Life Technologies). Cloning and transfection methods are well understood by practitioners of the art (Sambrook, Fritsch, Maniatis. Molecular Cloning: A Laboratory Manual). After 48 hours, cellular protein was extracted from the transfected cells and evaluated for PCC protein expression using Western analysis (Chandler, et al. 2010 Mol Ther 18:11-6). The results show that synPCCA1 is expressed at 140% the level of the wild type human PCCA gene (FIGS. 2 and 3) and also that synPCCA6 is transcribed and translated as well as or more efficiently than wild type human PCCA (FIGS. 2 and 3). Of interest, synPCCA6 expresses PCCA at levels close to the wild-type control CBA-PCCA even when expressed under the less potent EF1a promoters (FIGS. 2 and 3).

### Example 2

This example demonstrates AAV9 gene therapy in Pcca knock-out (Pcca^{-/-}) Mice.

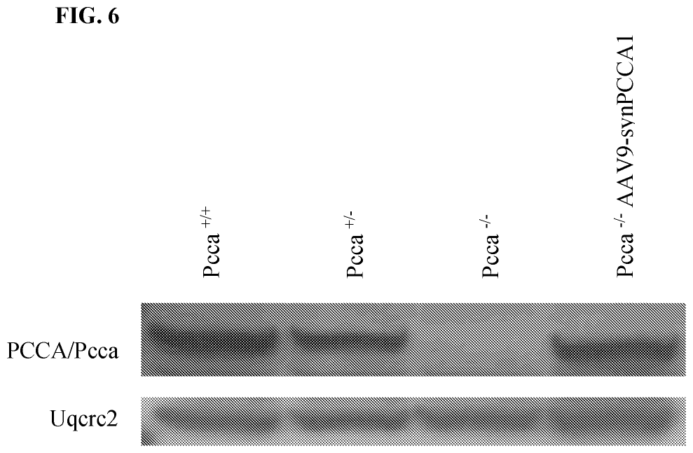

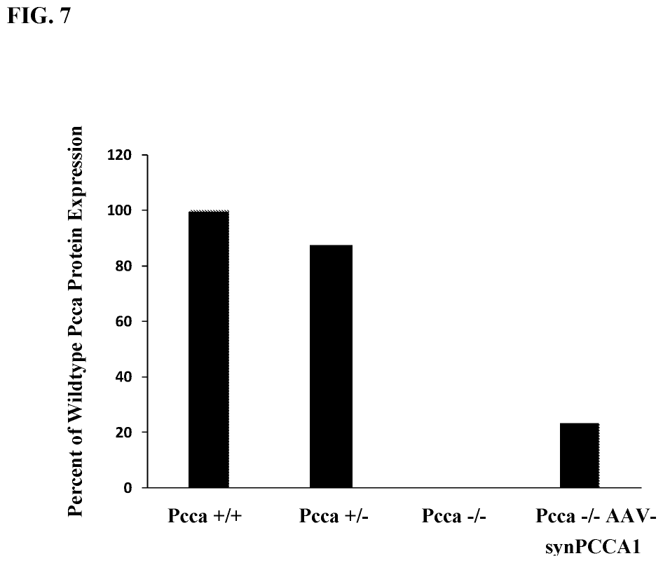

The promising expression data from both constructs led to the production of AAV9-CBA-synPCCA1 which was delivered to neonatal Pcca^{-/-} mice. As presented in FIG. 4, 50% of the Pcca^{-/-} mice that received the AAV lived to 30 days, and further had a wild type appearance, as compared to the untreated Pcca^{-/-} mice which had 100% mortality in early life. The surviving mice were sacrificed at 30 days for metabolic studies and to examine hepatic transgene expression. A substantial reduction in the disease related metabolite methylcitrate accompanied the rescue as seen in FIG. 5. Finally, a Western blot using murine livers, from wild-type mice (Pcca^{+/+} and Pcca^{+/-}), an untreated Pcca^{-/-} mouse, and a Pcca^{-/-} mouse treated with 3×10¹¹ VC of AAV9-CBA-synPCCA1, was performed. As seen in FIG. 6 and FIG. 7, the treated Pcca^{-/-} mouse displayed robust hepatic PCCA expression whereas the untreated Pcca^{-/-} mouse showed no hepatic murine Pcca expression. It should be noted that the antibody used for Western blotting can detect both human (PCCA) and murine (Pcca) enzymes.

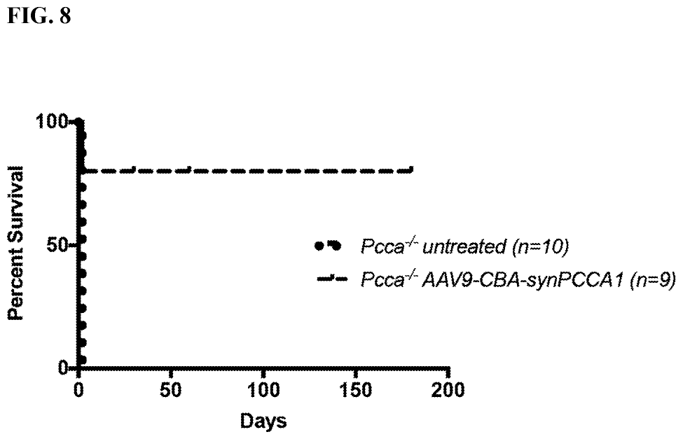

In a similar study, long term survival of neonatal AAV9-CBA-synPCCA1 treated Pcca^{-/-} mice was performed. Untreated Pcca^{-/-} (n=10) mice served as a control and were compared to Pcca^{-/-} mice (n=9) treated with 3×10¹¹ VC of AAV-CBA-synPCCA1 delivered by retroorbital injection at birth. As can be seen in FIG. 8, treated Pcca^{-/-} mice display a significant increase in survival to >150 days.

### Example 3

This example demonstrates the design of vectors expressing synPCCA1.

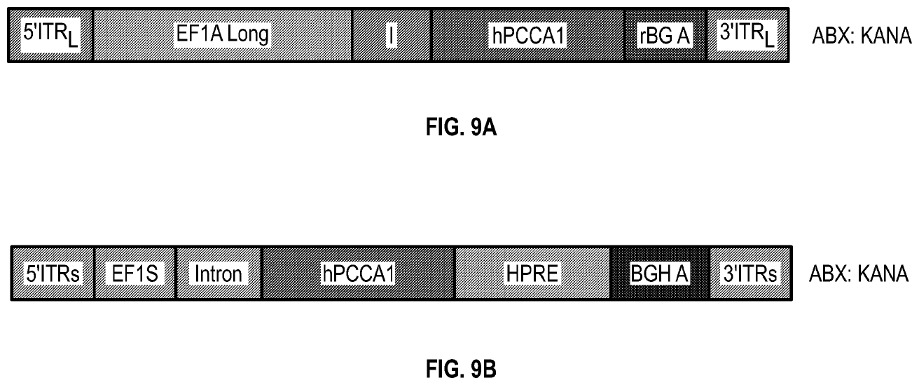

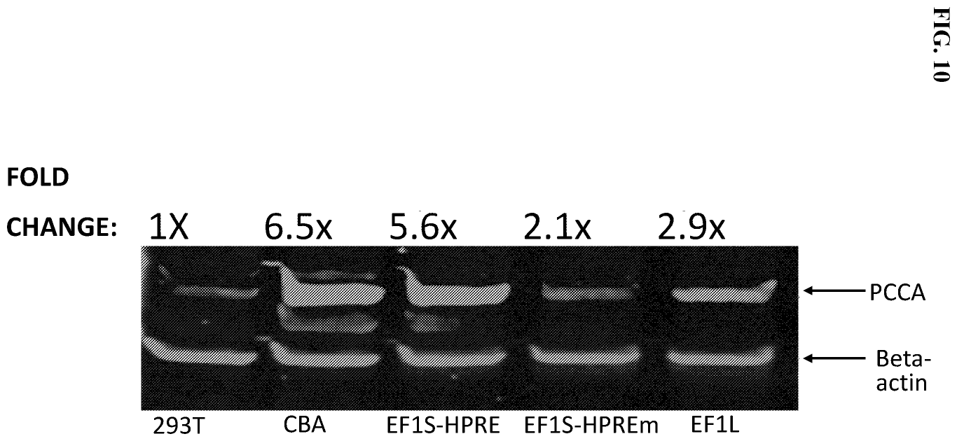

Next, a series of vectors was designed to express synPCCA1 from the long elongation factor 1 alpha promoter EF1 or EF1AS promoter in combination with a 3′ the HPRE. FIG. 9A shows a vector comprised of 145 base pair AAV2 inverted terminal repeats (5′ITRL and 3′ ITRL), the EF1AL promoter, an intron (I), the synPCCA1 gene, the rBGA. The production plasmid expresses the kanamycin resistance gene. FIG. 9B shows a vector comprised of 130 base pair AAV2 inverted terminal repeats (5′ITRS and 3′ ITRS), the EF1AS promoter, an intron (I), synPCCA1 gene, the HPRE, and the BGHA. The production plasmid expresses the kanamycin resistance gene.

### Example 4

This example demonstrates that the vectors of Example 3 are expressed in human cells.

The vectors were studied for expression in human cells. FIG. 10 presents a western blot showing PCCA protein expression in 293 cells, which are human transformed kidney cells, after transfection with transfected with AAV backbones expressing synPCCA1 under the control of various promoter/enhancer combinations. Cloning and transfection methods are well understood by practitioners of the art (Sambrook, Fritsch, Maniatis. Molecular Cloning: A Laboratory Manual). After 48 hours, cellular protein was extracted from the transfected cells and evaluated for PCCA protein expression using Western analysis (Chandler, et al. 2010 Mol Ther 18:11-6). PCCA=propionyl-CoA carboxylase alpha subunit, CBA=chicken beta actin, EF1a=elongation factor 1 alpha, EF1aS=elongation factor 1 alpha short. HPRE-hepatitis B post translation response element. HPREm-hepatitis B post translation response element, mutant. Beta-actin is the loading control. Compared to the untransfected cells (lane 1), the AAV plasmids expressed variably, with the CBA cassette (lane 2) showing 6.5× expression of the untransfected cells, the EF1S-HPRE cassette showing 5.6× expression of the untransfected cells (lane 3), the EF1S-HPREm cassette showing 2.1× expression of the untransfected cells (lane 4), and the EF1L cassette showing 2.9× the expression of untransfected cells. The results reveal that the EF1S-HPRE and EF1L cassettes substantially overexpress PCCA.

### Example 5

This example demonstrates the improved survival of Pcca^{-/-} mice treated with AAV9 synPCCA1 vectors as compared to untreated mice.

Next, AAV9 vectors were prepared using methods well known to practitioners (Chandler, et al. 2010 Mol Ther 18:11-6) and used to treat Pcca^{-/-} mice. FIG. 11 depicts survival in untreated Pcca^{-/-} (n=12) mice compared to Pcca^{-/-} mice (n=9) treated with 1×10¹¹ VC of AAV9-EF1aL-synPCCA1 (n=18), 1×10¹¹ VC of AAV9-EF1aS-synPCCA1-HPRE (n=15), or 4×10¹¹ VC of AAV9-EF1aS-synPCCA1-HPRE (n=5) delivered by retroorbital injection at birth. The treated Pcca^{-/-} mice display a significant increase in survival.

Animal studies were reviewed and approved by the National Human Genome Research Institute Animal User Committee. Retroorbital injections were performed on non-anesthetized neonatal mice, typically within several hours after birth. Viral particles were diluted to a total volume of 20 microliters with phosphate-buffered saline immediately before injection and were delivered into the retroorbital plexus using a 32-gauge needle.

Treatment with synPCCA1 polynucleotide delivered using an AAV (adeno-associated virus) rescued the Pcca^{-/-} mice from neonatal lethality (FIGS. 4,8,11), improved their growth, and lowered the levels of plasma methylcitrate in the blood (FIG. 5). This establishes the pre-clinical efficacy of synPCCA1 as a treatment for PA in vivo, including in other animal models, as well as in humans.

### Example 6

This example demonstrates the features of mouse models of PA.

Studies were initiated to develop systemic gene therapy as a treatment for PA, with the goal of targeting hepatocytes and cardiomyocytes for transgene expression. As a first step, improved alleles were generated using CRISPR/Cas9 genome editing to engineer mutations that are comparable to those seen in the patients, such as frameshift-stop and missense changes, into Pcca.

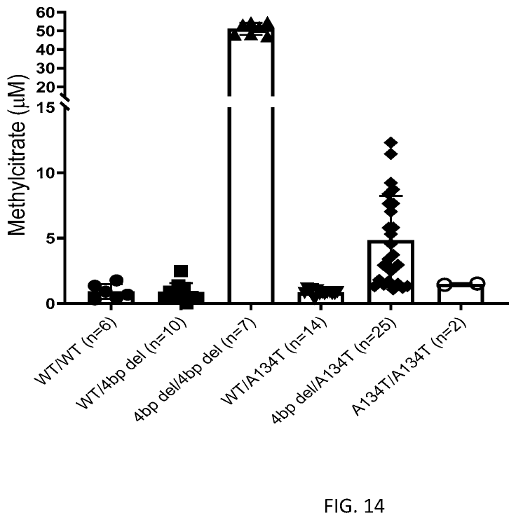

Among the mutations recovered, two were further studied: mPCCA^{p.Q133LfsX41}, caused by a 4 bp deletion in the Pcca gene (and also designated Pcca^{4bp del}, Pcca⁻, and Pcca K), and the missense mutation mPCCA^{p.A134T}. mPCCA^{p.Q133LfsX41/p.Q133LfsX41} mice, also designated Pcca^{-/-}, are null at the level of PCCA protein expression (cross reactive material, CRM−) and recapitulate the neonatal lethal form of PA in humans, while mPCCA^{p.A134T/p.A134T} and mPCCA^{p.Q133LfsX41/p.A134T} mice are fully viable as adults, yet display markedly reduced PCC activity and mildly elevated levels of 2-methylcitric acid, a metabolic biomarker of PA. Table 3 presents a summary of the mouse models. FIG. 14 presents plasma 2-methylcitrate levels in the various allelic combinations and in WT mice.

TABLE 3

| | | | | 2-methylcitrate |
|---|---|---|---|---|
| | | Phenotype of | | (2-MC) levels in |
| | Class of mutation | homozygote or | | homozygote or |
| Pcca | and effect on | compound | Expression, | compound |
| mutations | PCCA | heterozygote | protein | heterozygote |
| NM_144844.2 | Missense | Viable, normal | low levels of | Mildly increased, |
| c.400G > A | p.Ala134Thr | growth | PCCA | 2 mM |
| | p.A134T | | expression in | |
| | | | liver and heart | |
| NM_144844.2 | Deletion | Neonatal | no significant | Massively |
| c.398_401del | p.Gln133fs*44 | lethality, | PCCA | increased, |
| | p.Q133fs*44 | immediate | expression in | 40-50 mM on |
| | | | liver or heart | DOL1 |
| NM_144844.2 | Missense | Viable, normal | low levels of | Mildly increased, |
| c.400G > A | p.A134T | growth | PCCA | 2-10 mM |
| NM_144844.2 | Deletion | | expression in | |
| c.398_401del | p.Q133fs*44 | | liver and heart | |
| NM_144844.2: | Insertion | Neonatal | Not studied | Not studied |
| c.402dupT | p.Val135Cysfs*29 | lethality, | | |
| | | immediate | | |

### Example 7

This example demonstrates the design of a vector for the delivery of synPCCA (AAV9-EF1s-hPCCA-HPRE Final).

An expression cassette, EF1s-hPCCA-HPRE Final, was developed and improved from the parental vector (Ef1S-synPCCA1-HPRE parent) (FIG. 15A) by recloning the transgene insert between heterologous AAV2 ITRs of 130 bp and 141 bp to improve packaging and stability. CpG dinucleotides near the ITRs were removed from the parental cassette by deletion mutagenesis. The 5′ ITR is a variant and contains an 11 base pair deletion as compared to the wild type AAV2 ITR. Attachment L sites, or AttL sites, were likewise removed as summarized in FIG. 15A and Tables 4-5. The EF1s-hPCCA-HPRE Final expression cassette, from 3′ ITR to 5′ ITR, (shown in FIG. 15B) had the nucleotide sequence of SEQ ID NO: 41 (Table 6A).

The kanamycin resistant transgene was added, and the product was then scaled for production and packaged with an AAV9 capsid. The AAV9-EF1s-hPCCA-HPRE Final vector was purified by column chromatography and centrifugation. The AAV9-EF1s-hPCCA-HPRE Final vector including the kanamycin resistance gene comprised SEQ ID NO: 42 (Table 6B).

The AAV9-EF1s-hPCCA-HPRE Final vector is an AAV9 vector expressing a functional human codon optimized cDNA encoding PCCA, under control of the EFIS promoter. In humans, endogenous PCCA protein is ubiquitously expressed, therefore the therapeutic transgene cassette was designed with a constitutive promoter to enable wide expression. The AAV9 capsid was selected to further enable hepatic and cardiac transduction.

TABLE 4

| AAV9-EF1s-hPCCA-HPRE parent | |
|---|---|
| vector | AAV9-EF1s-hPCCA-HPRE Final vector |
| 5′ and 3′ ITR are 130 bp | 5′ ITR is 130 bp variant and 3′ ITR is 141 |
| | bp |
| Contains CpG enriched extra restriction | Extra CpG enriched extra restriction sites |
| sites on either side of ITR | are removed |
| | Will mitigate innate immune |
| | reactivity which in part depends on |
| | CpG content |
| Contains AttL sites outside ITRs | AttL sites are removed |
| Could increase reverse packaging at | |
| scale which could interfere with | |
| large scale production and facilitate | |
| reverse packaging of the plasmid | |
| backbone into the final AAV | |
| product | |
| AAV9-EF1s-hPCCA-HPRE parent | AAV9-EF1s-hPCCA-HPRE Final vector |
| vector | |
| Produced in small scale cell culture systems | Scaled for production |
| only | Large scale growth and yield |
| | desirable for clinical translation |
| | In vitro assay using a HepG2 PCCA knock |
| | out cell line to establish potency |

TABLE 5
Components of AAV9-EF1s-hPCCA-HPRE Final vector

| Element | Length (base pairs) | Comment |
|---|---|---|
| 5′ ITR | 130 | AAV2-derived |
| EFIS | 231 | Core elongation factor 1 alpha promoter |
| Intron | 165 | Synthetic |
| hPCCA | 2187 | Codon optimized human PCCA, contains Kozak |
| HPRE | 726 | 3′ mRNA stability element, hepatitis B-derived |
| poly A | 225 | Bovine growth hormone |
| 3′ ITR | 141 | AAV2 |
| antibiotic | — | Kanamycin |
| Ori | — | F1 |

TABLE 6A
EF1s-hPCCA-HPRE
FINAL CASSETTE (from 5′ ITR to 3′ ITR)
CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCGTAGGTACCGGCTC
CGGTGCCCGTCAGTGGGCAGAGCGCACATCGCCCACAGTCCCCGAGAAGT
TGGGGGGAGGGGTCGGCAATTGAACCGGTGCCTAGAGAAGGTGGCGCGGG
GTAAACTGGGAAAGTGATGTCGTGTACTGGCTCCGCCTTTTTCCCGAGGG
TGGGGGAGAACCGTATATAAGTGCAGTAGTCGCCGTGAACGTTCTTTTTC
GCAACGGGTTTGCCGCCAGAACACAGGTCAGATCAGATCTTTGTCGATCC
TACCATCCACTCGACACACCCGCCAGCGGCCGCGTTGGTATCAAGGTTAC
AAGACAGGTTTAAGGAGACCAATAGAAACTGGGCATGTGGAGACAGAGAA
GACTCTTGGGTTTCTGATAGGCACTGACTCTCTTCCTTTGTCCTGTTCCC
ATTTCAGAAGCTTCCGAGCTCTCGAATTCGAGCTCGGTACCTCGCGTGCA
TCTAGATAATCCACCATGGCAGGGTTCTGGGTCGGCACCGCACCTCTGGT
CGCCGCAGGACGCAGGGGAAGATGGCCTCCACAGCAGCTGATGCTGAGCG
CAGCCCTGAGGACCCTGAAGCACGTGCTGTACTATTCTAGGCAGTGCCTG
ATGGTCAGCCGCAACCTGGGCAGCGTGGGATACGACCCTAATGAGAAGAC
ATTCGATAAAATCCTGGTGGCTAACCGCGGCGAAATCGCATGCCGAGTGA
TTCGGACCTGTAAGAAAATGGGGATCAAGACAGTCGCCATTCACAGCGAC
GTGGATGCCAGCAGCGTCCATGTGAAGATGGCAGACGAGGCCGTCTGCGT
GGGACCAGCCCCTACATCTAAAAGTTACCTGAACATGGATGCTATCATGG
AAGCAATTAAGAAAACTAGGGCCCAGGCTGTGCACCCTGGCTATGGGTTC
CTGAGC
EF1s-hPCCA-HPRE
FINAL CASSETTE (from 5′ ITR to 3′ ITR)
GAGAATAAGGAATTTGCACGATGTCTGGCAGCTGAGGACGTGGTCTTTAT
CGGACCAGATACACATGCTATTCAGGCAATGGGCGACAAGATCGAGTCCA
AACTGCTGGCCAAGAAAGCTGAAGTGAATACTATCCCCGGGTTCGACGGA
GTGGTCAAGGATGCAGAGGAAGCCGTGAGAATCGCCAGGGAGATTGGCTA
CCCTGTGATGATTAAGGCATCTGCCGGCGGGGGAGGCAAAGGGATGAGGA
TCGCCTGGGACGATGAGGAAACTCGCGATGGATTTCGACTGTCTAGTCAG
GAAGCAGCCAGCAGCTTCGGCGACGATAGGCTGCTGATCGAGAAGTTCAT
TGACAACCCCCGCCACATCGAAATTCAGGTGCTGGGGGATAAACATGGAA
ACGCCCTGTGGCTGAATGAGCGGGAATGTAGCATTCAGCGGAGAAATCAG
AAGGTGGTCGAGGAAGCTCCTTCCATCTTTCTGGACGCCGAGACAAGGCG
CGCTATGGGAGAACAGGCTGTCGCACTGGCCAGAGCTGTGAAATACTCCT
CTGCCGGCACTGTCGAGTTCCTGGTGGACAGCAAGAAAAACTTCTATTTT
CTGGAAATGAACACCCGGCTGCAGGTCGAGCACCCAGTGACTGAATGCAT
TACCGGGCTGGATCTGGTCCAGGAGATGATCAGAGTGGCCAAGGGATACC
CCCTGCGACATAAACAGGCTGACATCCGGATTAACGGCTGGGCAGTCGAG
TGTCGGGTGTACGCCGAAGATCCATATAAGTCTTTCGGACTGCCCAGTAT
TGGCCGACTGTCACAGTATCAGGAGCCTCTGCACCTGCCAGGCGTCAGAG
TGGACAGCGGCATCCAGCCTGGGTCCGACATCTCTATCTACTATGATCCA
ATGATCAGCAAGCTGATTACATACGGCTCCGATCGGACTGAGGCCCTGAA
AAGAATGGCAGACGCCCTGGATAACTATGTCATTAGAGGGGTGACCCATA
ATATCGCTCTGCTGAGAGAAGTCATCATTAACTCCAGGTTCGTGAAGGGA
GACATCAGCACCAAATTTCTGTCCGACGTGTACCCCGATGGCTTCAAGGG
GCACATGCTGACAAAGTCTGAGAAAAATCAGCTGCTGGCTATCGCAAGTT
CACTGTTCGTGGCATTTCAGCTGCGGGCCCAGCATTTTCAGGAGAACAGT
AGAATGCCCGTGATCAAGCCTGACATTGCAAATTGGGAACTGAGTGTCAA
GCTGCACGATAAAGTGCATACCGTGGTCGCTTCAAACAATGGCAGCGTGT
TCAGCGTCGAGGTGGACGGGTCTAAACTGAACGTGACCAGTACATGGAAT
CTGGCCTCACCACTGCTGTCAGTCAGCGTGGATGGCACACAGCGCACTGT
GCAGTGCCTGAGCCGGGAGGCAGGAGGAAACATGAGTATTCAGTTTCTGG
GGACTGTCTATAAGGTGAACATCCTGACCAGGCTGGCTGCAGAACTGAAT
AAGTTCATGCTGGAGAAAGTGACCGAAGACACAAGCTCCGTGCTGCGCTC
ACCAATGCCAGGAGTGGTCGTGGCCGTCAGCGTGAAGCCAGGGGATGCAG
TGGCTGAGGGACAGGAGATTTGCGTGATTGAGGCTATGAAAATGCAGAAC
AGCATGACCGCAGGAAAGACTGGCACCGTGAAAAGCGTGCATTGTCAGGC
TGGGGATACTGTCGGGGAAGGGGATCTGCTGGTGGAACTGGAGTGAAGAC
GCGTGGTACCTCTAGAGTCGACCCGGGCGGCCTCGAGATAACAGGCCTAT
TGATTGGAAAGTTTGTCAACGAATTGTGGGTCTTTTGGGGTTTGCTGCCC
CTTTTACGCAATGTGGATATCCTGCTTTAATGCCTTTATATGCATGTATA
CAAGCAAAACAGGCTTTTACTTTCTCGCCAACTTACAAGGCCTTTCTCAG
TAAACAGTATATGACCCTTTACCCCGTTGCTCGGCAACGGCCTGGTCTGT
GCCAAGTGTTTGCTGACGCAACCCCCACTGGTTGGGGCTTGGCCATAGGC
CATCAGCGCATGCGTGGAACCTTTGTGTCTCCTCTGCCGATCCATACTGC
GGAACTCCTAGCCGCTTGTTTTGCTCGCAGCAGGTCTGGAGCAAACCTCA
TCGGGACCGACAATTCTGTCGTACTCTCCCGCAAGTATACATCGTTTCCA
TGGCTGCTAGGCTGTGCTGCCAACTGGATCCTGCGCGGGACGTCCTTTGT
TTACGTCCCGTCGGCGCTGAATCCCGCGGACGACCCCTCCCGGGGCCGCT
TGGGGCTCTACCGCCCGCTTCTCCGTCTGCCGTACCGTCCGACCACGGGG
CGCACCTCTCTTTACGCGGACTCCCCGTCTGTGCCTTCTCATCTGCCGGA
CCGTGTGCACTTCGCTTCACCTCTGCACGTCGCATGGAGGCCACCGTGAA
CGCCCACCGGAACCTGCCCAAGGTCTTGCATAAGAGGACTCTTGGACTTT
CAGCAATGTCATCGATATCGTCGACTCGCTGATCAGCCTCGACTGTGCCT
TCTAGTTGCCAGCCATCTGTTGTTTGCCCCTCCCCCGTGCCTTCCTTGAC
CCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGG
CAGGACAGCAAGGGGGAGGATTGGGAAGACAATAGCAGGCATGCTGGGGA
TGCGGTGGGCTCTATGGCTTCTGAGGCGGAAAGAACCAGCTGGGGCTCGA
CTAGACTAGTCCTGCAGGTACGGCCGCaggaacccctagtgatggagttg
gccactccctctctgcgcGCTCGCTCGCTCACTGAGGCCGGGCGACCAAA
GGTCGCCCGACGCCCGGGCTTTGCCCGGGCGGCCTCAGTGAGCGAGCGAG
CGCGCAGCTGCCTGCAGG (SEQ ID NO: 41)

TABLE 6B
AAV9-EF1s-hPCCA-HPRE Final vector including the kanamycin resistance gene

ATGTCCTGCAGGCAGCTGCGCGCTCGCTCGCTCACTGAGGCCGCCCGGGCGTCGGGCGACCTTTGGTCGCC
CGGCCTCAGTGAGCGAGCGAGCGCGCAGAGAGGGAGTGGCCAACTCCATCACTAGGGGTTCCTGCGGCC
GTAGGTACCGGCTCCGGTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGGGAAAGTGATGTCGTGT
ACTGGCTCCGCCTTTTTCCCGAGGGTGGGGGAGAACCGTATATAAGTGCAGTAGTCGCCGTGAACGTTCTT
TTTCGCAACGGGTTTGCCGCCAGAACACAGGTCAGATCAGATCTTTGTCGATCCTACCATCCACTCGACACA
CCCGCCAGCGGCCGCGTTGGTATCAAGGTTACAAGACAGGTTTAAGGAGACCAATAGAAACTGGGCATGT
GGAGACAGAGAAGACTCTTGGGTTTCTGATAGGCACTGACTCTCTTCCTTTGTCCTGTTCCCATTTCAGAAG
CTTCCGAGCTCTCGAATTCGAGCTCGGTACCTCGCGTGCATCTAGATAATCCACCATGGCAGGGTTCTGGGT
CGGCACCGCACCTCTGGTCGCCGCAGGACGCAGGGGAAGATGGCCTCCACAGCAGCTGATGCTGAGCGCA
GCCCTGAGGACCCTGAAGCACGTGCTGTACTATTCTAGGCAGTGCCTGATGGTCAGCCGCAACCTGGGCAG
CGTGGGATACGACCCTAATGAGAAGACATTCGATAAAATCCTGGTGGCTAACCGCGGCGAAATCGCATGC
CGAGTGATTCGGACCTGTAAGAAAATGGGGATCAAGACAGTCGCCATTCACAGCGACGTGGATGCCAGCA
GCGTCCATGTGAAGATGGCAGACGAGGCCGTCTGCGTGGGACCAGCCCCTACATCTAAAAGTTACCTGAA
CATGGATGCTATCATGGAAGCAATTAAGAAAACTAGGGCCCAGGCTGTGCACCCTGGCTATGGGTTCCTGA
GCGAGAATAAGGAATTTGCACGATGTCTGGCAGCTGAGGACGTGGTCTTTATCGGACCAGATACACATGCT
ATTCAGGCAATGGGCGACAAGATCGAGTCCAAACTGCTGGCCAAGAAAGCTGAAGTGAATACTATCCCCG
GGTTCGACGGAGTGGTCAAGGATGCAGAGGAAGCCGTGAGAATCGCCAGGGAGATTGGCTACCCTGTGA
TGATTAAGGCATCTGCCGGCGGGGGAGGCAAAGGGATGAGGATCGCCTGGGACGATGAGGAAACTCGCG
ATGGATTTCGACTGTCTAGTCAGGAAGCAGCCAGCAGCTTCGGCGACGATAGGCTGCTGATCGAGAAGTT
CATTGACAACCCCCGCCACATCGAAATTCAGGTGCTGGGGGATAAACATGGAAACGCCCTGTGGCTGAAT
GAGCGGGAATGTAGCATTCAGCGGAGAAATCAGAAGGTGGTCGAGGAAGCTCCTTCCATCTTTCTGGACG
CCGAGACAAGGCGCGCTATGGGAGAACAGGCTGTCGCACTGGCCAGAGCTGTGAAATACTCCTCTGCCGG
CACTGTCGAGTTCCTGGTGGACAGCAAGAAAAACTTCTATTTTCTGGAAATGAACACCCGGCTGCAGGTCG
AGCACCCAGTGACTGAATGCATTACCGGGCTGGATCTGGTCCAGGAGATGATCAGAGTGGCCAAGGGATA
CCCCCTGCGACATAAACAGGCTGACATCCGGATTAACGGCTGGGCAGTCGAGTGTCGGGTGTACGCCGAA
GATCCATATAAGTCTTTCGGACTGCCCAGTATTGGCCGACTGTCACAGTATCAGGAGCCTCTGCACCTGCCA
GGCGTCAGAGTGGACAGCGGCATCCAGCCTGGGTCCGACATCTCTATCTACTATGATCCAATGATCAGCAA
GCTGATTACATACGGCTCCGATCGGACTGAGGCCCTGAAAAGAATGGCAGACGCCCTGGATAACTATGTCA
TTAGAGGGGTGACCCATAATATCGCTCTGCTGAGAGAAGTCATCATTAACTCCAGGTTCGTGAAGGGAGAC
ATCAGCACCAAATTTCTGTCCGACGTGTACCCCGATGGCTTCAAGGGGCACATGCTGACAAAGTCTGAGAA
AAATCAGCTGCTGGCTATCGCAAGTTCACTGTTCGTGGCATTTCAGCTGCGGGCCCAGCATTTTCAGGAGA
ACAGTAGAATGCCCGTGATCAAGCCTGACATTGCAAATTGGGAACTGAGTGTCAAGCTGCACGATAAAGT
GCATACCGTGGTCGCTTCAAACAATGGCAGCGTGTTCAGCGTCGAGGTGGACGGGTCTAAACTGAACGTG
ACCAGTACATGGAATCTGGCCTCACCACTGCTGTCAGTCAGCGTGGATGGCACACAGCGCACTGTGCAGTG
CCTGAGCCGGGAGGCAGGAGGAAACATGAGTATTCAGTTTCTGGGGACTGTCTATAAGGTGAACATCCTG
ACCAGGCTGGCTGCAGAACTGAATAAGTTCATGCTGGAGAAAGTGACCGAAGACACAAGCTCCGTGCTGC
GCTCACCAATGCCAGGAGTGGTCGTGGCCGTCAGCGTGAAGCCAGGGGATGCAGTGGCTGAGGGACAGG
AGATTTGCGTGATTGAGGCTATGAAAATGCAGAACAGCATGACCGCAGGAAAGACTGGCACCGTGAAAAG
CGTGCATTGTCAGGCTGGGGATACTGTCGGGGAAGGGGATCTGCTGGTGGAACTGGAGTGAAGACGCGT
GGTACCTCTAGAGTCGACCCGGGCGGCCTCGAGATAACAGGCCTATTGATTGGAAAGTTTGTCAACGAATT
GTGGGTCTTTTGGGGTTTGCTGCCCCTTTTACGCAATGTGGATATCCTGCTTTAATGCCTTTATATGCATGTA
TACAAGCAAAACAGGCTTTTACTTTCTCGCCAACTTACAAGGCCTTTCTCAGTAAACAGTATATGACCCTTTA
CCCCGTTGCTCGGCAACGGCCTGGTCTGTGCCAAGTGTTTGCTGACGCAACCCCCACTGGTTGGGGCTTGG
CCATAGGCCATCAGCGCATGCGTGGAACCTTTGTGTCTCCTCTGCCGATCCATACTGCGGAACTCCTAGCCG
CTTGTTTTGCTCGCAGCAGGTCTGGAGCAAACCTCATCGGGACCGACAATTCTGTCGTACTCTCCCGCAAGT
ATACATCGTTTCCATGGCTGCTAGGCTGTGCTGCCAACTGGATCCTGCGCGGGACGTCCTTTGTTTACGTCC
CGTCGGCGCTGAATCCCGCGGACGACCCCTCCCGGGGCCGCTTGGGGCTCTACCGCCCGCTTCTCCGTCTG
CCGTACCGTCCGACCACGGGGCGCACCTCTCTTTACGCGGACTCCCCGTCTGTGCCTTCTCATCTGCCGGAC
CGTGTGCACTTCGCTTCACCTCTGCACGTCGCATGGAGGCCACCGTGAACGCCCACCGGAACCTGCCCAAG
GTCTTGCATAAGAGGACTCTTGGACTTTCAGCAATGTCATCGATATCGTCGACTCGCTGATCAGCCTCGACT
GTGCCTTCTAGTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGCATGCTGGGGATGCGGTG
GGCTCTATGGCTTCTGAGGCGGAAAGAACCAGCTGGGGCTCGACTAGACTAGTCCTGCAGGTACGGCCGC
AGGAACCCCTAGTGATGGAGTTGGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGCGAGCGCGCAGCTGCCTGCAG
GGGCGCCTGATGCGGTATTTTCTCCTTACGCATCTGTGCGGTATTTCACACCGCATACGTCAAAGCAACCAT
AGTACGCGCCCTGTAGCGGCGCATTAAGCGCGGCGGGTGTGGTGGTTACGCGCAGCGTGACCGCTACACT
TGCCAGCGCCTTAGCGCCCGCTCCTTTCGCTTTCTTCCCTTCCTTTCTCGCCACGTTCGCCGGCTTTCCCCGTC
AAGCTCTAAATCGGGGGCTCCCTTTAGGGTTCCGATTTAGTGCTTTACGGCACCTCGACCCCAAAAAACTTG
ATTTGGGTGATGGTTCACGTAGTGGGCCATCGCCCTGATAGACGGTTTTTCGCCCTTTGACGTTGGAGTCCA
CGTTCTTTAATAGTGGACTCTTGTTCCAAACTGGAACAACACTCAACTCTATCTCGGGCTATTCTTTTGATTT
ATAAGGGATTTTGCCGATTTCGGTCTATTGGTTAAAAAATGAGCTGATTTAACAAAAATTTAACGCGAATTT
TAACAAAATATTAACGTTTACAATTTTATGGTGCACTCTCAGTACAATCTGCTCTGATGCCGCATAGTTAAGC
CAGCCCCGACACCCGCCAACACCCGCTGACGCGCCCTGACGGGCTTGTCTGCTCCCGGCATCCGCTTACAG
ACAAGCTGTGACCGTCTCCGGGAGCTGCATGTGTCAGAGGTTTTCACCGTCATCACCGAAACGCGCGAGAC
GAAAGGGCCTCGTGATACGCCTATTTTTATAGGTTAATGTCATGAACAATAAAACTGTCTGCTTACATAAAC
AGTAATACAAGGGGTGTTATGAGCCATATTCAACGGGAAACGTCGAGGCCGCGATTAAATTCCAACATGG
ATGCTGATTTATATGGGTATAAATGGGCTCGCGATAATGTCGGGCAATCAGGTGCGACAATCTATCGCTTG
TATGGGAAGCCCGATGCGCCAGAGTTGTTTCTGAAACATGGCAAAGGTAGCGTTGCCAATGATGTTACAG
ATGAGATGGTCAGACTAAACTGGCTGACGGAATTTATGCCTCTTCCGACCATCAAGCATTTTATCCGTACTC
CTGATGATGCATGGTTACTCACCACTGCGATCCCCGGAAAAACAGCATTCCAGGTATTAGAAGAATATCCT
GATTCAGGTGAAAATATTGTTGATGCGCTGGCAGTGTTCCTGCGCCGGTTGCATTCGATTCCTGTTTGTAAT
TGTCCTTTTAACAGCGATCGCGTATTTCGTCTCGCTCAGGCGCAATCACGAATGAATAACGGTTTGGTTGAT
GCGAGTGATTTTGATGACGAGCGTAATGGCTGGCCTGTTGAACAAGTCTGGAAAGAAATGCATAAACTTTT
GCCATTCTCACCGGATTCAGTCGTCACTCATGGTGATTTCTCACTTGATAACCTTATTTTTGACGAGGGGAA
ATTAATAGGTTGTATTGATGTTGGACGAGTCGGAATCGCAGACCGATACCAGGATCTTGCCATCCTATGGA
ACTGCCTCGGTGAGTTTTCTCCTTCATTACAGAAACGGCTTTTTCAAAAATATGGTATTGATAATCCTGATAT
GAATAAATTGCAGTTTCATTTGATGCTCGATGAGTTTTTCTAATCAGAATTGGTTAATTGGTTGTAACACTG
GCAGAGCATTACGCTGACTTGACGGGACGGCGCAAGCTCATGACCAAAATCCCTTAACGTGAGTTTTCGTT
CCACTGAGCGTCAGACCCCGTAGAAAAGATCAAAGGATCTTCTTGAGATCCTTTTTTTCTGCGCGTAATCTG
CTGCTTGCAAACAAAAAAACCACCGCTACCAGCGGTGGTTTGTTTGCCGGATCAAGAGCTACCAACTCTTTT
TCCGAAGGTAACTGGCTTCAGCAGAGCGCAGATACCAAATACTGTTCTTCTAGTGTAGCCGTAGTTAGGCC
ACCACTTCAAGAACTCTGTAGCACCGCCTACATACCTCGCTCTGCTAATCCTGTTACCAGTGGCTGCTGCCA
GTGGCGATAAGTCGTGTCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTATCTTTATAGTCCTGTCGGGT
TTCGCCACCTCTGACTTGAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCAC (SEQ ID NO: 42)

### Example 8

This example demonstrates the expression of PCCA encoded by the AAV9-EF1s-hPCCA-HPRE Final vector in a human liver (HepG2) PCCA knockout (KO) cell line.

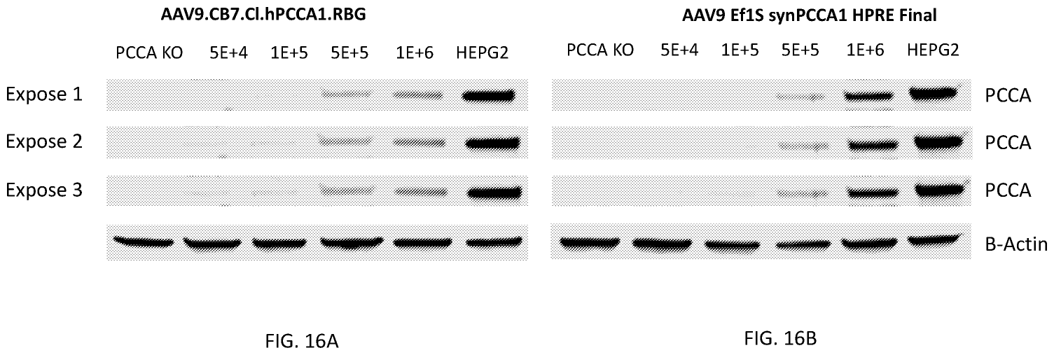

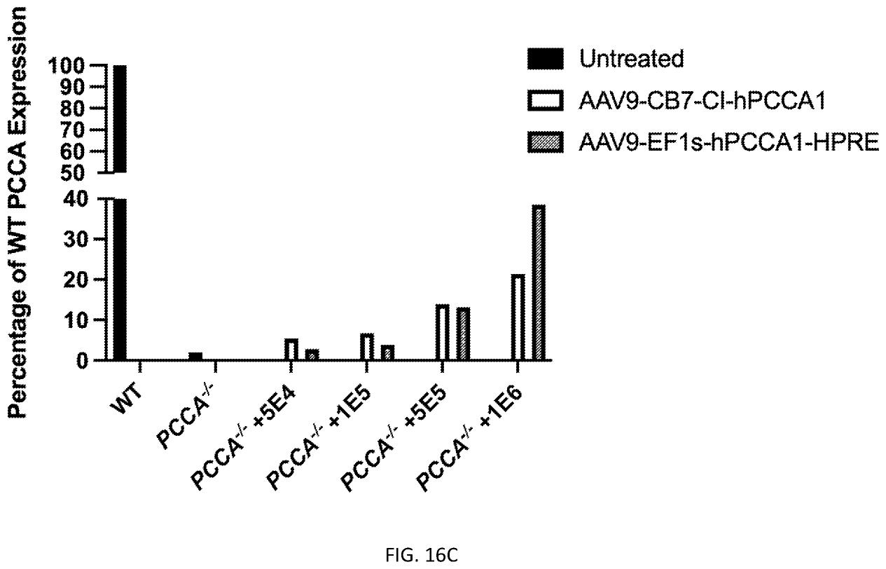

The AAV9-EF1s-hPCCA-HPRE Final vector of Example 7 was tested for activity and expression in a human liver (HepG2) PCCA knock-out cell line (FIGS. 16A-16C).

A deletion mutation in PCCA was engineered in a human hepatocyte derived cell line (HEPG2) to create the PCCA KO cell line, and used to test the in vitro potency of the AAV9-EF1s-hPCCA-HPRE Final vector applied at varying moiety of infections ranging from 5×10⁴ to 1×10⁶. After 48 hours, the infected PCCA KO cells were harvested and lysed, and 25 μg of total cellular protein was subjected to Western analysis using a PCCA polyclonal antibody. Beta-actin was used as a loading control. A dose response with increasing PCCA after exposure to varying doses of AAV9-EF1s-hPCCA-HPRE Final was apparent and approximated the proof-of-concept AAV9 vector (AAV9.CB7.C1.hPCCA1.RBG) of Example 3 that used the CMV enhanced chicken beta actin promoter to drive the expression of synPCCA1 (FIGS. 16A-16C).

### Example 9

This example demonstrates the efficacy and dose response of the AAV9-EF1s-hPCCA-HPRE Final vector in a mouse model of PA.

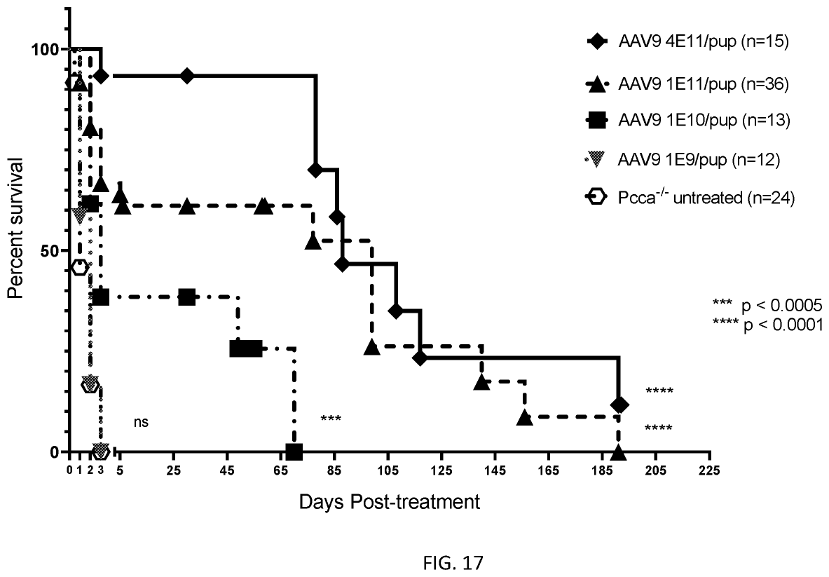

The AAV9-EF1s-hPCCA-HPRE Final vector of Example 7 was delivered by retroorbital injection to treat the severely affected neonatal lethal PA mice of Table 3 over doses ranging from 8.3e11 vector genomes/kilogram (vg/kg) to 3.3e14 vg/kg. The doses for the experiment shown in FIG. 17 are shown in Table 7. The results are shown in FIG. 17.

The three higher doses provided rescue from neonatal lethality, and substantially prolonged the life of the mutant mice in a dose response manner. The control Pcca^{-/-} animals (untreated/PBS treated) died shortly after birth as did those dosed with 8.3×10¹¹ vg/kg. The animals dosed at 8.3×10¹² vg/kg had a mean survival of ˜41 days, which increased with dose: those in the 8.3×10¹³ vg/kg cohorts survived for ˜85 days, while animals in the highest dose group of 3.3×10¹⁴ vg/kg lived even longer, with a mean survival past 112.5 days.

TABLE 7

| Dose per pup | Approximate dose |
|---|---|
| vector genome | vector genome per kg |
| per pup (vg/pup) | (vg/kg) |
| 1 × 10⁹ | 8.3 × 10¹¹ |
| 1 × 10¹⁰ | 8.3 × 10¹² |
| 1 × 10¹¹ | 8.3 × 10¹³ |
| 4 × 10¹¹ | 3.3 × 10¹⁴ |

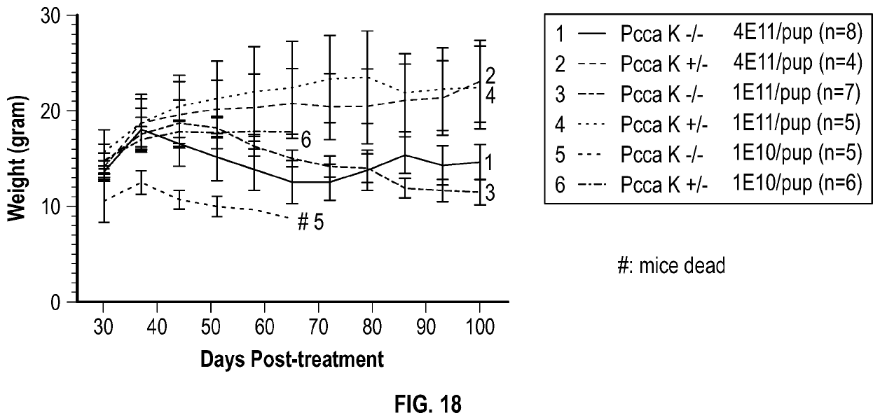

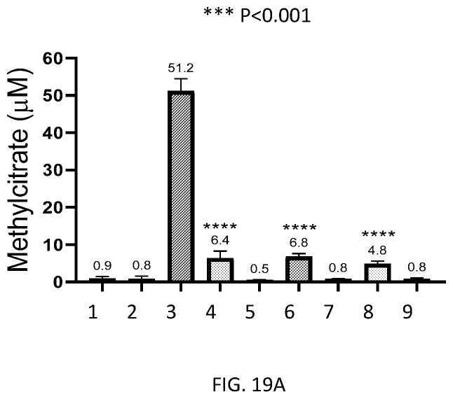

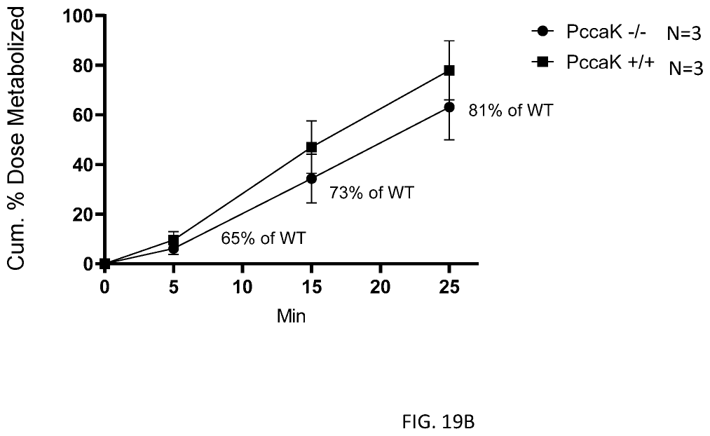

Animals dosed with the AAV9-EF1s-hPCCA-HPRE Final vector were bled to measure a biomarker response at day 30, and then sacrificed to assess transgene expression and vector biodistribution. On day 30, the weights of the treated Pcca^{-/-} mice were not different than treated control littermates (FIG. 18). Treatment of Pcca^{-/-} mice with the AAV9-EF1s-hPCCA-HPRE Final vector resulted in a substantial reduction in the plasma levels of 2-MC as compared to the untreated animals at doses of 8.3×10¹³ and 3.3×10¹⁴ vg/kg, with milder changes on day 30 in the lowest dose 8.3×10¹² vg/kg cohort (FIG. 19A).

AAV9-EF1s-hPCCA-HPRE Final provided effective rescue from neonatal lethality displayed by mPCCA^{p.Q133LfsX41/p.Q133LfsX41} mice, with survivors manifesting improvements in growth (FIG. 18), as well as metabolic parameters including reduced plasma 2-methylcitrate levels (FIG. 19A) increased 1-C713 propionate oxidation (FIG. 19B).

The doses for the experiments shown in FIGS. 18 and 19B are shown in Table 8. The doses for the experiment shown in FIG. 19A are shown in Table 9.

TABLE 8

| Dose per pup vector | |
|---|---|
| genome per pup | Approximate dose vector |
| (vg/pup) | genome per kg (vg/kg) |
| 1 × 10¹⁰ | 8.3 × 10¹² |
| 1 × 10¹¹ | 8.3 × 10¹³ |
| 4 × 10¹¹ | 3.3 × 10¹⁴ |

TABLE 9

| | Number ON | |
|---|---|---|
| | X-AXIS OF | Dose of Ef1S synPCCA1 |
| GROUP | FIG. 19A | HPRE AAV9 |
| 1 Pcca^{+/+} untreated | 6 | |
| 2Pcca^{+/-} untreated | 10 | |
| 3Pcca^{-/-} untreated | 7 | |
| 4Pcca^{-/-} | 5 | 1e10 GC/pup |
| 5Pcca^{+/-} | 2 | 1e10 GC/pup |
| 6Pcca^{-/-} | 11 | 1e11 GC/pup |
| 7Pcca^{+/-} | 4 | 1e11 GC/pup |
| 8Pcca^{-/-} | 14 | 4e11 GC/pup |
| 9Pcca^{+/-} | 6 | 4e11 GC/pup |

### Example 10

This example demonstrates hepatic and cardiac transgene expression in a mouse model of PA after treatment with the AAV9-EF1s-hPCCA-HPRE Final vector.

In the PA patients, the target tissues are the liver and heart, and therefore, the survey was focused on hepatic and cardiac studies in mice treated with the AAV9-EF1s-hPCCA-HPRE Final vector.

Neonatal Pcca^{-/-} mice were treated with the AAV9-EF1s-hPCCA-HPRE Final vector with a dose of 1e11 GC/pup delivered by retro-orbital injection.

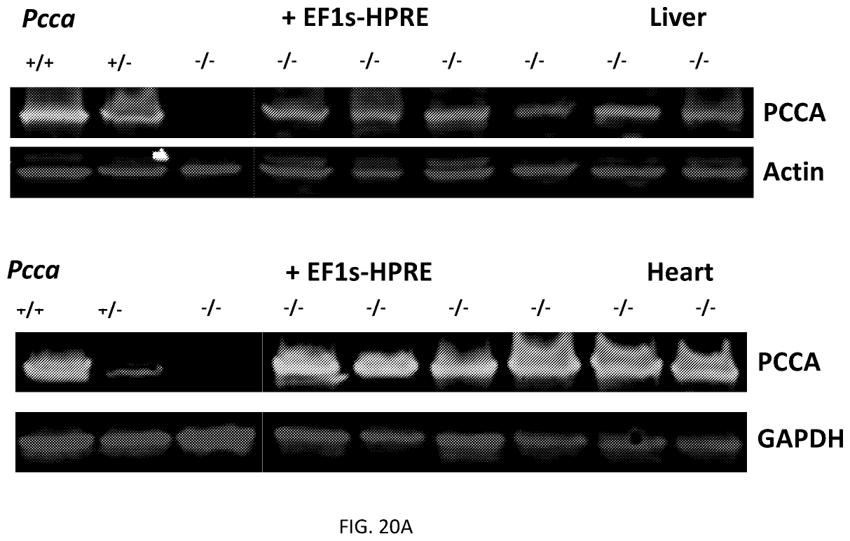

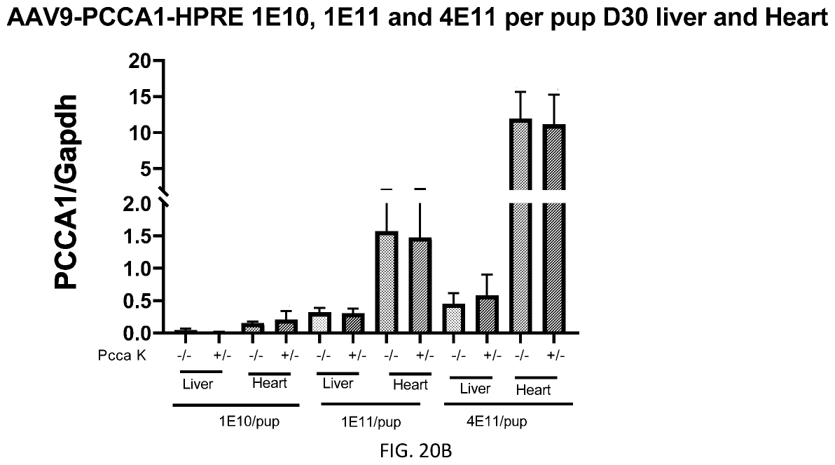

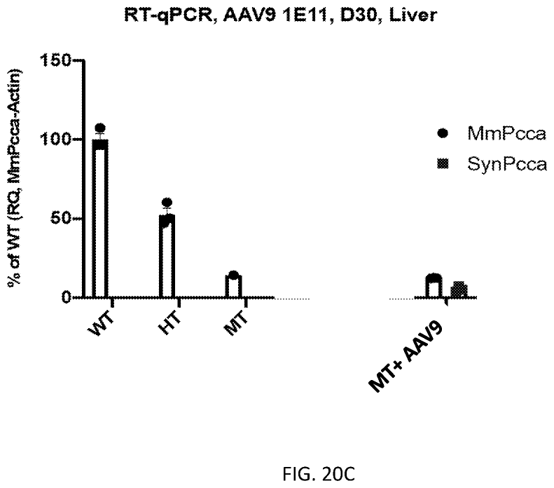

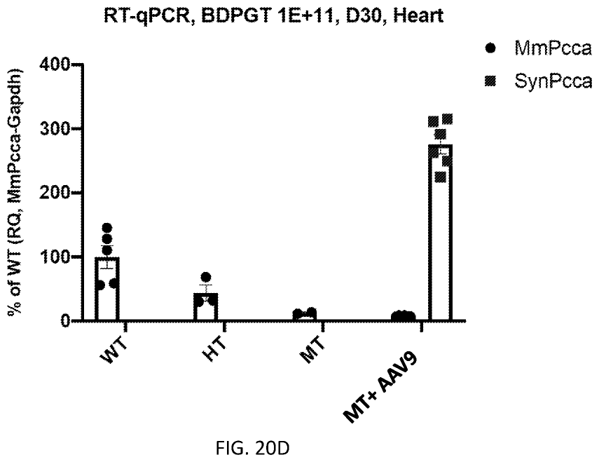

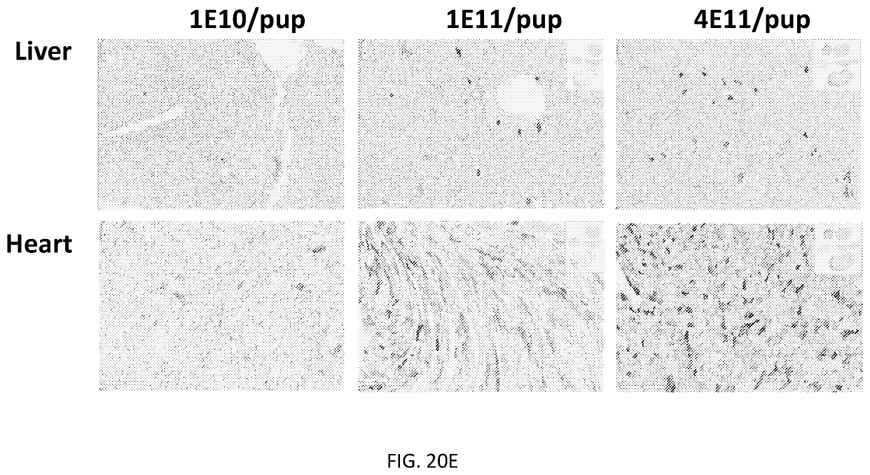

Expression of PCCA in liver and heart was measured by Western blot on DOL 30. PCCA expression was detected in the heart and liver of the treated mice (FIG. 20A).

AAV9-EF1s-hPCCA-HPRE Final treated mPCCA^{p.Q133LfsX41/p.Q133LfsX41} mice exhibited robust PCCA transgene expression in the liver and heart as measured by Western blotting (FIG. 20A). Using digital droplet (ddPCR) with probes designed to detect the synPCCA1 transgene and the endogenous Gapdh locus, the copy numbers of the vector synPCCA1 transgene were assessed at Day 30. An abundance of vector genomes in the liver and heart was apparent (FIG. 20B). synPCCA1 mRNA expression was detected by RT-qPCR in the same tissues (FIG. 20C and FIG. 20D), and synPCCA1 mRNA expression was histologically detailed using RNA in situ hybridization with a probe specifically designed to detect synPCCA1 (FIG. 20E). The mRNA levels were normalized to endogenous glyceraldehyde 3-phosphate dehydrogenase (Gapdh) expression and protein levels were normalized to endogenous beta actin levels. The AAV encoded PCCA mRNA varied between ˜15-25% of WT Pcca levels and the protein expression was at ˜40% of the wild-type murine PCCA level.

FIG. 20E shows an RNA in situ hybridization study to detect the expression of synPCCA1 in Pcca^{-/-} mice after treatment with AAV9-Ef1S-synPCCA1-HPRE Final on DOL 30 (Doses: 1e10 GC/pup, 1e11 GC/pup, 4e11 GC/pup). Liver and heart tissues were removed, fixed in formalin and stained with an RNA probe designed to specifically hybridize to the synPCCA mRNA. The dark staining cells in the liver are hepatocytes or cardiomyocytes in the heart, and increase in number with a higher dose of AAV. This study confirms the cellular targeting of the AAV9-Ef1S-synPCCA1-HPRE Final to the specific cell types needed to treat humans with PA.

This vector, AAV9-EF1s-hPCCA-HPRE Final, and data obtained from mPCCA^{p.Q133LfsX41/p.Q133LfsX41} (Pcca^{-/-}) neonatal mouse model experiments establish a new AAV gene therapy for PA caused by PCCA mutations, and further provide first-in-human (FIH) dose ranges for efficacy.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A polynucleotide expression cassette comprising:
(a) synthetic propionyl-CoA carboxylase subunit a (PCCA) polynucleotide (synPCCA) selected from the group consisting of:
(i) a polynucleotide comprising the nucleic acid sequence of any one of SEQ ID NOs: 2-7; and
(ii) a polynucleotide comprising a nucleic acid sequence with at least 80% identity to the nucleic acid sequence of any one of SEQ ID NOs: 2-7 which encodes the polypeptide of SEQ ID NO:8 and has equivalent or greater expression in a host relative to expression of any one of SEQ ID NOs: 2-7 or SEQ ID NO:1, wherein the polynucleotide does not have the nucleic acid sequence of SEQ ID NO: 1; and
(b) any one or more of the following features:
(i) a 3′ inverted terminal repeat (3′ ITR) with a length greater than 135 base pairs (bp) and less than 145 bp;
(ii) fewer CpG dinucleotides as compared to a nucleic acid sequence of any one or more of SEQ ID NOs: 9-12 and 35-40; and
(iii) fewer restriction enzyme recognition sites as compared to a nucleic acid sequence of any one or more of SEQ ID NOs: 9-12 and 35-40.

2. The polynucleotide expression cassette of claim 1, further comprising any one or more of the following features:
(i) a 5′ ITR nucleotide sequence at least 90% identical to nucleotides 1 to 130 of SEQ ID NO: 41;
(ii) a core elongation factor 1 alpha (EF1S) promoter nucleotide sequence at least 90% identical to nucleotides 131 to 361 of SEQ ID NO: 41;
(iii) an intron nucleotide sequence at least 90% identical to nucleotides 362 to 526 of SEQ ID NO: 41;
(iv) a hepatitis B post translation response element (HPRE) nucleotide sequence at least 90% identical to nucleotides 2714 to 3439 of SEQ ID NO: 41;
(v) a polyA nucleotide sequence at least 90% identical to nucleotides 3440 to 3664 of SEQ ID NO: 41; and
(vi) a 3′ ITR nucleotide sequence at least 90% identical to nucleotides 3665 to 3805 of SEQ ID NO: 41.

3. The polynucleotide expression cassette of claim 1, further comprising all of the following features:
(i) a 5′ ITR nucleotide sequence at least 90% identical to nucleotides 1 to 130 of SEQ ID NO: 41;
(ii) a EF1S promoter nucleotide sequence at least 90% identical to nucleotides 131 to 361 of SEQ ID NO: 41;
(iii) an intron nucleotide sequence at least 90% identical to nucleotides 362 to 526 of SEQ ID NO: 41;
(iv) a HPRE nucleotide sequence at least 90% identical to nucleotides 2714 to 3439 of SEQ ID NO: 41;
(v) a polyA nucleotide sequence at least 90% identical to nucleotides 3440 to 3664 of SEQ ID NO: 41; and
(vi) a 3′ ITR nucleotide sequence at least 90% identical to nucleotides 3665 to 3805 of SEQ ID NO: 41.

4. The polynucleotide expression cassette of claim 1, wherein the synPCCA polynucleotide comprises a synPCCA1 nucleotide sequence at least 90% identical to SEQ ID NO: 2.

5. The polynucleotide expression cassette of claim 1, comprising any one or more of the following features:
(i) a 5′ ITR nucleotide sequence consisting of a nucleotide sequence at least 90% identical to nucleotides 1 to 130 of SEQ ID NO: 41;
(ii) a EF1S promoter nucleotide sequence consisting of a nucleotide sequence at least 90% identical to nucleotides 131 to 361 of SEQ ID NO: 41;
(iii) a intron nucleotide sequence consisting of a nucleotide sequence at least 90% identical to nucleotides 362 to 526 of SEQ ID NO: 41;
(iv) a HPRE nucleotide sequence consisting of a nucleotide sequence at least 90% identical to nucleotides 2714 to 3439 of SEQ ID NO: 41;
(v) a polyA nucleotide sequence consisting of a nucleotide sequence at least 90% identical to nucleotides 3440 to 3664 of SEQ ID NO: 41; and
(vi) a 3′ ITR nucleotide sequence consisting of a nucleotide sequence at least 90% identical to nucleotides 3665 to 3805 of SEQ ID NO: 41.

6. The polynucleotide expression cassette of claim 1, comprising all of the following features:
(i) a 5′ ITR nucleotide sequence consisting of a nucleotide sequence at least 90% identical to nucleotides 1 to 130 of SEQ ID NO: 41;
(ii) a EF1S promoter nucleotide sequence consisting of a nucleotide sequence at least 90% identical to nucleotides 131 to 361 of SEQ ID NO: 41;
(iii) a intron nucleotide sequence consisting of a nucleotide sequence at least 90% identical to nucleotides 362 to 526 of SEQ ID NO: 41;
(iv) a HPRE nucleotide sequence consisting of a nucleotide sequence at least 90% identical to nucleotides 2714 to 3439 of SEQ ID NO: 41;
(v) a polyA nucleotide sequence consisting of a nucleotide sequence at least 90% identical to nucleotides 3440 to 3664 of SEQ ID NO: 41; and
(vi) a 3′ ITR nucleotide sequence consisting of a nucleotide sequence at least 90% identical to nucleotides 3665 to 3805 of SEQ ID NO: 41.

7. The polynucleotide expression cassette of claim 1, wherein the synPCCA polynucleotide consists of a synPCCA1 nucleotide sequence at least 90% identical to SEQ ID NO: 2.

8. The polynucleotide expression cassette of claim 1, comprising a nucleotide sequence at least 90% identical to SEQ ID NO: 41 or SEQ ID NO: 42.

9. The polynucleotide expression cassette of claim 1, consisting of a nucleotide sequence at least 90% identical to the nucleotide sequence of SEQ ID NO: 41 or SEQ ID NO: 42.

10. A recombinant expression vector comprising the polynucleotide expression cassette of claim 1.

11. The recombinant expression vector of claim 10, wherein the vector is a recombinant adeno-associated virus (rAAV), wherein the rAAV comprises an AAV capsid and a vector genome packaged therein.

12. The rAAV according to claim 11, wherein the vector is comprised of the structure in FIG. 15B.

13. The rAAV according to claim 11, wherein the AAV capsid is from an AAV of serotype 1, 2, 3, 4, 5, 6, 7, 8, 9, 10, 11, 12, 13, rh 10, hu37 or Anc, or mutants thereof.

14. The rAAV according to claim 13, wherein the AAV capsid is from an AAV of serotype 9.

15. A composition comprising (a)(i) the polynucleotide expression cassette of claim 1 or (ii) a recombinant expression vector comprising the polynucleotide expression cassette of (i), and (b) a pharmaceutically acceptable carrier.

16. A method for treating a disease or condition mediated by propionyl-CoA carboxylase in a subject, the method comprising administering (i) the polynucleotide expression cassette of claim 1, (ii) a recombinant expression vector comprising the polynucleotide expression cassette of (i, or (iii) a composition comprising (i) or (ii) and a pharmaceutically acceptable carrier to a subject, wherein the subject has a disease or condition mediated by propionyl-CoA carboxylase.

17. The method of claim 16, wherein the disease or condition is propionic acidemia (PA).

18. The method of claim 16, wherein the vector is an rAAV and is formulated for administration at a dose of 1×10¹¹ to 1×10¹⁴ genome copies (GC)/kg.

19. The method of claim 16, wherein the vector is an rAAV and is formulated for administration as a single dose of rAAV.

20. The method of claim 16, wherein the vector is an rAAV and is formulated for administration as multiple doses of rAAV.

21. An in vitro method comprising administering to a cell of a subject, or a population of cells of the subject, the polynucleotide expression cassette of claim 1, wherein the polynucleotide expression cassette is inserted into the cell of the subject, or the population of cells of the subject, via genome editing on the cell of the subject using a nuclease selected from the group of zinc finger nucleases (ZFNs), transcription activator-like effector nucleases (TALENs), the clustered regularly interspaced short palindromic repeats (CRISPR/cas system) and meganuclease re-engineered homing endonucleases on the cell from the subject, or the population of cells of the subject.

22. The method of claim 16, wherein the polynucleotide expression cassette, recombinant expression vector, rAAV, or composition is formulated for subcutaneous, intramuscular, intradermal, intraperitoneal, or intravenous administration.

23. A nucleic acid consisting of (a) a nucleotide sequence that is at least 12 but no more than 35 contiguous nucleotides of any one of SEQ ID NO: 2-7; (b) a nucleotide sequence that is at least 90% identical to at least 12 but no more than 35 contiguous nucleotides of any one of SEQ ID NO: 2-7; or (c) a nucleotide sequence that is complementary to (a) or (b).

24. The nucleic acid of claim 23, further comprising a detectable label.

25. A collection of nucleic acids comprising two or more of the nucleic acids of claim 23.

26. The collection according to claim 25, wherein the two or more nucleic acids comprise two or more different nucleic acids.

27. An in vitro method for detecting the expression of synPCCA nucleic acid by cells, the method comprising:
(a) contacting a sample comprising nucleic acid from the cells with the nucleic acid of claim 23 or a collection comprising two or more thereof, thereby forming a complex; and
(b) detecting the complex, wherein detection of the complex is indicative of the expression of synPCCA nucleic acid by the cells.

28. The method of claim 27, wherein the method is carried out prior to administering (i) the polynucleotide expression cassette of claim 1, (ii) a recombinant expression vector comprising the polynucleotide expression cassette of (i, or (iii) a composition comprising (i) or (ii) and a pharmaceutically acceptable carrier to a subject.

29. The method of claim 27, wherein the cells are from a subject that has been treated with (i) the polynucleotide expression cassette of the polynucleotide expression cassette of claim 1, (ii) a recombinant expression vector comprising the polynucleotide expression cassette of (i), or (iii) a composition comprising (i) or (ii) and a pharmaceutically acceptable carrier, and wherein detection of the complex is indicative of the expression of synPCCA nucleic acid by the cells of the subject.

30. The method of claim 27, wherein the cells are heart cells or liver cells.

31. The method of claim 27, wherein the cells are hepatocytes or cardiomyocytes.

32. The method of claim 27, wherein the cells are cells of a cell line.

33. The method of claim 27, wherein the cells are cells of a heart cell line or a liver cell line.

34. The method of claim 27, wherein the cells are cells of a hepatocyte cell line or a cardiomyocyte cell line.

35. The method of claim 34, wherein the cells are cells of a hepatocyte cell line which does not express one or both of endogenous PCCA mRNA and PCCA protein.

36. The method of claim 27, further comprising quantifying the synPCCA nucleic acid expressed by the cells.

37. The method of claim 27, further comprising quantifying the cells that express the synPCCA nucleic acid.
